SEQUENCE LISTING (26 sequences)

SEQ ID NO: 1 - DNA, 19 bp, Escherichia coli
tggggagcataacaggatt

SEQ ID NO: 2 - DNA, 19 bp, Escherichia coli
  misc_feature (10)..(10): deoxy Uracil (dU)
tggggagcanaacaggatt

SEQ ID NO: 3 - DNA, 19 bp, Escherichia coli
  misc_feature (10)..(10): abasic site
tggggagcanaacaggatt

SEQ ID NO: 4 - DNA, 19 bp, Escherichia coli
aatcctgttatgctcccca

SEQ ID NO: 5 - DNA, 19 bp, Escherichia coli
  misc_feature (10)..(10): Abasic site
aatcctgttntgctcccca

SEQ ID NO: 6 - DNA, 20 bp, Homo sapiens
ttagggtttagggtttaggg

SEQ ID NO: 7 - DNA, 20 bp, Homo sapiens
ttagggattagggattaggg

SEQ ID NO: 8 - DNA, 20 bp, Homo sapiens
  misc_feature (7)..(7): deoxy Uracil (dU)
ttagggnttagggnttaggg

SEQ ID NO: 9 - DNA, 20 bp, Homo sapiens
  misc_feature (7)..(7): abasic site
ttagggnttagggnttaggg

SEQ ID NO: 10 - DNA, 20 bp, Homo sapiens
ccctaaaccctaaaccctaa

SEQ ID NO: 11 - DNA, 20 bp, Homo sapiens
ccctaatccctaatccctaa

SEQ ID NO: 12 - DNA, 20 bp, Homo sapiens
  misc_feature (7)..(7): deoxy Uracil (dU)
ccctaanccctaanccctaa

SEQ ID NO: 13 - DNA, 20 bp, Homo sapiens
  misc_feature (7)..(7): abasic site
ccctaanccctaanccctaa

SEQ ID NO: 14 - RNA, 71 nt, Escherichia coli
gcuguggauauacgagggcuugaugucuaucaggcgcguuuugaccaucuucgucugauuauugagcaaaa

SEQ ID NO: 15 - RNA, 71 nt, Escherichia coli
uuuugcucaauaaucagacgaagauggucaaaacgcgccugauagacaucaagcccucguauauccacagc

SEQ ID NO: 16 - DNA, 26 bp, Escherichia coli
cgttttgaccatcttcgtctgattaa

SEQ ID NO: 17 - DNA, 26 bp, Escherichia coli
  misc_feature (8)..(8): para-TINA
cgttttgnccntcttcgtctgnttaa

SEQ ID NO: 18 - DNA, 20 bp, Escherichia coli
gggcttgatgtctatcaggc

SEQ ID NO: 19 - DNA, 20 bp, Escherichia coli
  misc_feature (8)..(8): para-TINA
gggcttgntgtctntcnggc

SEQ ID NO: 20 - DNA, 30 bp, Escherichia coli
  misc_feature (6)..(6): c or abasic site
tattgnacaatgggngcaagnctgatgcag

SEQ ID NO: 21 - DNA, 28 bp, Escherichia coli
  misc_feature (6)..(6): c or abasic site
tattgnacaatgggngcaagnctgatgc

SEQ ID NO: 22 - DNA, 26 bp, Escherichia coli
  misc_feature (6)..(6): c or abasic site
tattgnacaatgggngcaagnctgat

SEQ ID NO: 23 - DNA, 24 bp, Escherichia coli
  misc_feature (6)..(6): c or abasic site
tattgnacaatgggngcaagnctg

SEQ ID NO: 24 - DNA, 22 bp, Escherichia coli
  misc_feature (6)..(6): c or abasic site
tattgnacaatgggngcaagnc

SEQ ID NO: 25 - DNA, 20 bp, Escherichia coli
  misc_feature (6)..(6): c or abasic site
tattgnacaatgggngcaag

SEQ ID NO: 26 - DNA, 18 bp, Escherichia coli
  misc_feature (6)..(6): c or abasic site
tattgnacaatgggngca

(19) United States
(12) Patent Application Publication
(10) Pub. No.: US 2013/0230856 A1
(43) Pub. Date: Sep. 5, 2013

(54) CAPTURE OF TARGET DNA AND RNA BY PROBES COMPRISING INTERCALATOR MOLECULES

(71) Applicants: Uffe Vest Schneider, Valby (DK); Nina Johnk, Lyngby (DK); Jan Gorm Lisby, Vekso (DK)

(72) Inventors: Uffe Vest Schneider, Valby (DK); Nina Johnk, Lyngby (DK); Jan Gorm Lisby, Vekso (DK)

(73) Assignee: QUANTIBACT A/S, Hvidovre (DK)

(21) Appl. No.: 13/881,714

(22) PCT Filed: Oct. 27, 2011
(86) PCT No.: PCT/DK2011/000120

§ 371 Date: Apr. 25, 2013

Related U.S. Application Data
(60) Provisional application No. 61/407,122, filed on Oct. 27, 2010.

(30) Foreign Application Priority Data
Oct. 27, 2010 (DK) PA 2010 70455

(51) Int. Cl. C12Q 1/68

(52) U.S. Cl. CPC C12Q 1/6883
USPC 435/6.11; 536/24.3

(57) ABSTRACT

The present invention relates to a technology for specific capture of single stranded target Polynucleotide by a complementary probe comprising one or more intercalator molecules. The method further involves removal of one or more types of bases in the single stranded target Polynucleotide prior to interaction with the complementary probe. This results in generation of one or more abasic sites which can interact with and/or into where the intercalator molecule can be inserted.

### TECHNICAL FIELD

The present invention relates to a technology for molecular diagnostics comprising specific capture of single stranded target polynucleotide which may be made of naturally occurring nucleotides or which may be made of nucleotides which are not known to occur naturally or any mixture thereof, such as e.g. DNA and/or RNA, by a complementary probe comprising one or more intercalator molecules. The method further involves removal of one or more types of bases from the target polynucleotide which may be made of naturally occurring nucleotides or which may be made of nucleotides which are not known to occur naturally or any mixture thereof, such as e.g. in DNA and/or RNA, prior to interaction with the complementary probe.

### BACKGROUND OF THE INVENTION

Polymerase chain reaction (PCR) is a widely used technique in molecular genetics and diagnostics that permits the analysis of any short sequence of DNA even in samples containing only a low level of DNA. PCR is used to amplify selected sections of DNA or RNA for analysis.

Several limitations are associated with PCR based diagnostics. Firstly, due to the extremely high sensitivity of PCR, contamination from non-template PCR present in the laboratory environment (e.g. from bacteria, viruses, and human DNA) presents a significant problem. Second, amplification of rare targets is often inhibited by amplification of abundant targets. In addition, the DNA polymerase can introduce mistakes. The polymerases used in PCR often lack 3′ to 5′ exonuclease activity such as Taq polymerase. This enzyme lacks the ability to correct misincorporated nucleotides.

Further limitations by known methods for analysis and detection of short sequences of nucleotides is that they generally involves at least one step of purification and that their specificity is not sufficient for the identification of a single base mis-match.

Covalent attachment of hydrophobic structures, known as intercalators, intercalating molecules or intercalator molecules, has previously been used for modification of nucleic acids. Several DNA intercalators including INA, TINA and AMANY have previously been described [3, 4, 5]. Pyrene has previously been paired against an abasic site in duplex DNA [6].

### SUMMARY OF THE INVENTION

The present invention relates to a method for capture of polynucleotide such as single stranded target polynucleotide, such as e.g. DNA or RNA, from a sample comprising the steps of:
i) removal of one or more of the types of bases A, T, U, C or G, 5-hydroxymethyl-dC, 5-methylcytosine (m⁵C), pseudouridine (Ψ), dihydrouridine (D), inosine (I), 7-methylguanosine (m⁷G), hypoxanthine, xanthine and their 2′-O-Methyl-derivatives and/or N-Methyl-derivatives from said target polynucleotide thereby generating one or more abasic sites and
ii) capture of said target polynucleotide with a complementary probe comprising one or more intercalator molecules which are inserted into the backbone structure of a polynucleotide probe and which fit morphologically into an abasic site of a complementary polynucleotide target sequence;
   wherein said target polynucleotide may be made of naturally occurring nucleotides or of nucleotides which are not known to occur naturally or any mixture thereof, saod target polynucleotide may thus e.g. be made of nucleotides such as those selected from the group consisting of RNA, α-L-RNA, β-D-RNA, 2′-R-RNA, DNA, LNA, PNA, PMO, TNA, GNA, oligonucleotide N3′→P5′ phosphoramidates, BNA, α-L-LNA, HNA, MNA, ANA, CAN, INA, CeNA, (2′-NH)-TNA, (3′—NH)-TNA, α-L-Ribo-LNA, α-L-Xylo-LNA, 13-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-OR-RNA, 2′-AE-RNA, and combinations and modifications thereof.

In a preferred embodiment the present invention relates to a method for capture of single stranded target polynucleotide comprising the steps of:
(i) providing double stranded target polynucleotide which may be made of naturally occurring nucleotides or which may be made of nucleotides which are not known to occur naturally or any mixture thereof, such as e.g. DNA;
(ii) destabilisation of said double stranded target polynucleotide, such as e.g. DNA, by removal of one or more of the types of bases from said double stranded target polynucleotide, such as e.g. DNA, thereby generating one or more abasic sites;
(iii) denaturing of said destabilized double stranded target polynucleotide, such as e.g. DNA, to single stranded target polynucleotide, such as e.g. DNA, and
(iv) capture of said single stranded target polynucleotide, such as e.g. DNA, with a complementary polynucleotide probe, such as e.g. a DNA probe, comprising one or more intercalator molecules which are inserted into the backbone structure of a polynucleotide probe and which fit morphologically into an abasic site of a complementary polynucleotide target sequence.

In a specific embodiment, the present invention relates to a method for capture of polynucleotide such as single stranded target DNA or RNA from a sample comprising the steps of
i) removal of one or more of the types of bases A, T, U, C or G from said target polynucleotide such as DNA or RNA thereby generating one or more abasic sites and
ii) capture of said target polynucleotide such as DNA or RNA with a complementary probe comprising one or more intercalator molecules which can be inserted into one or more of the one ore more abasic sites.

In another specific embodiment, the present invention relates to a method for capture of single stranded target DNA comprising the steps of
(i) providing double stranded target DNA
(ii) destabilisation of said double stranded target DNA by removal of one or more of the types of bases A, T, U, C or G from said double stranded target DNA thereby generating one or more abasic sites
(iii) denaturing of said destabilized double stranded target DNA to single stranded target DNA and
(iv) capture of said single stranded target DNA with a complementary DNA probe comprising one or more intercalator molecules which can be inserted into the one ore more abasic sites.

The present invention further relates to polynucleotide probes which may be made of naturally occurring nucleotides or which may be made of nucleotides which are not known to occur naturally or any mixture thereof, said probe comprising two or more intercalator molecules suitable for capture of single stranded target polynucleotide which may be made of naturally occurring nucleotides or which may be made of nucleotides which are not known to occur naturally or any mixture thereof, such as e.g. DNA and/or RNA.

### DEFINITIONS AND ABBREVIATIONS

The terms ‘nucleobase’ or ‘base’ refer to a group of nitrogen-based molecules that are required to form polynucleotides in that they provide the molecular structure necessary for the hydrogen bonding of complementary nucleotide strands and are key components in the formation of stable polynucleotide molecules. Non-limiting examples of nucleobases are selected from the group consisting of A, G, C, T, U, 5-hydroxymethyl-dC, 5-methylcytosine (m⁵C), pseudouridine (Ψ), dihydrouridine (D), inosine (I), 7-methylguanosine (m⁷G), hypoxanthine, xanthine and their 2′-O-Methyl-derivatives and/or N-Methyl-derivatives.

Of these nucleobases:

A: adenine. Adenine forms a base pair with e.g. thymine.
T: Thymine. Thymine forms a base pair with e.g. adenine.
G: guanine. Guanine forms a base pair with e.g. cytosine.
C: cytosine. Cytosine forms a base pair with e.g. guanine.
U: uracil. Uracil forms base pair with e.g. adenine

The terms ‘polynucleotide sequence’ designates sequence of nucleotides which occur naturally or which are not known to occur naturally. Non-limiting examples of such nucleotides are selected from the group consisting of RNA, α-L-RNA, β-D-RNA, 2′-R-RNA, DNA, LNA, PNA, PMO, TNA, GNA, nucleotide N3′→P5′ phosphoramidates, BNA, α-L-LNA, HNA, MNA, ANA, CAN, INA, CeNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-OR-RNA, 2′-AE-RNA, and combinations and modifications thereof.

The terms ‘naturally occurring polynucleotides’ and ‘naturally occurring polynucleotide sequence’ designates a polynucleotide sequence consisting of nucleotides which occur in nature, such as comprising RNA (e.g. α-L-RNA, β-D-RNA, 2′-R-RNA) and/or DNA.

The term ‘polynucleotide sequence not known to occur naturally’ designates such polynucleotide sequences which are not known from nature, i.e. polynucleotide sequences which are made of one or more analogue(s) of the naturally occurring nucleotides. Non-limiting examples of such analogues are selected from the group consisting of LNA, PNA, PMO, TNA, GNA, oligonucleotide N3′→P5′ phosphoramidates, BNA, α-L-LNA, HNA, MNA, ANA, CAN, INA, CeNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, and combinations and modifications thereof. Such ‘oligonucleotide not known to occur naturally’ may be made of nucleotide analogues not known to occur naturally as the only kind of nucleotides, or it may be a mixture of nucleotide moieties known from nature and nucleotide analogues not known to occur naturally.

The term ‘LNA’ refers to Locked Nucleic Acid is often referred to as inaccessible RNA and is a modified RNA nucleotide. The ribose moiety of an LNA nucleotide is modified with an extra bridge connecting the 2′ oxygen and 4′ carbon. The bridge “locks” the ribose in the 3′-endo (North) conformation:

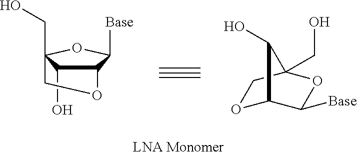

The term ‘PNA’ refers to Peptide Nucleic Acid wherein the backbone is composed of repeating N-(2-aminoethyl)-glycine units linked by peptide bonds. The various purine and pyrimidine bases are linked to the backbone by methylene carbonyl:

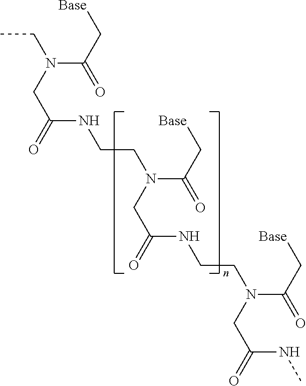

The term ‘PMO’ designates morpholino oligomers in which the nucleic acid bases are bound to morpholine rings instead of e.g. the ribose rings used by RNA. The morpholine rings are linked through phosphorodiamidate and the backbone of a PMO is thus made from these modified subunits:

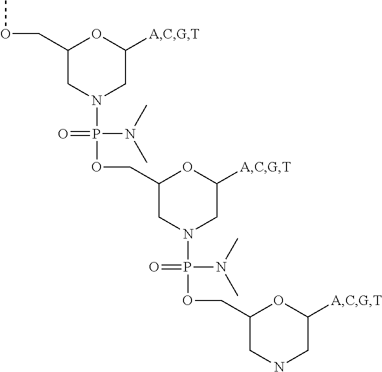

The term ‘TNA’ designates threose nucleic acid which is a polymer similar to DNA or RNA but differing in the composition of its “backbone” in that TNA's backbone is composed of repeating threose units linked by phosphodiester bonds:

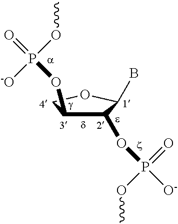

The term ‘GNA’ designates glycol nucleic acid which is a polymer similar to DNA or RNA but differing in the composition of its “backbone” in that is composed of repeating glycerol units linked by phosphodiester bonds:

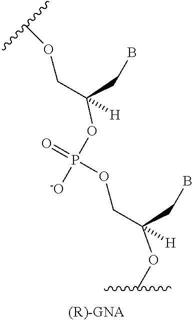

The term ‘BNA’ refers to an oligonucleotide comprising a BNA nucleoside which may e.g. be selected from the below group of nucleosides:

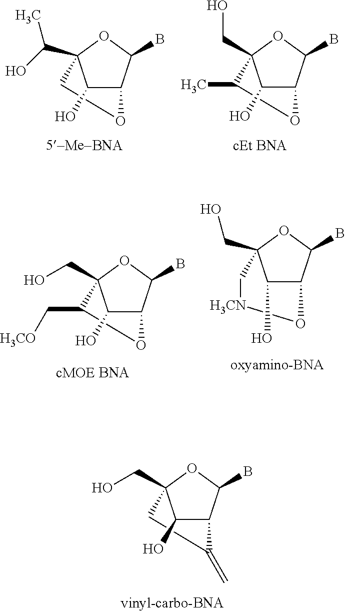

The term ‘α-L-LNA’ refers to an oligonucleotide comprising a ‘α-L-LNA nucleoside’ which may e.g. be selected from the below group of nucleosides:

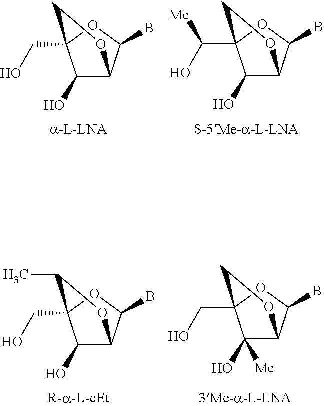

The term ‘other constrained nucleotide’ refers to an oligonucleotide comprising a constrained nucleoside which may e.g. be selected from the below group of nucleosides:

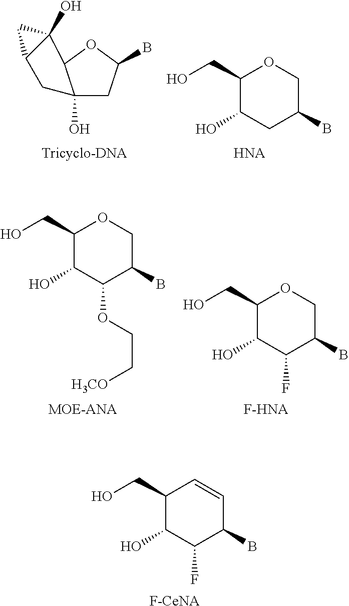

The term ‘oligonucleotide N3′→P5′ phosphoramidates’ refers to a oligonucleotide comprising a N3′→P5′ phosphoramidate oligonucleotide such as the ones are outlined below:

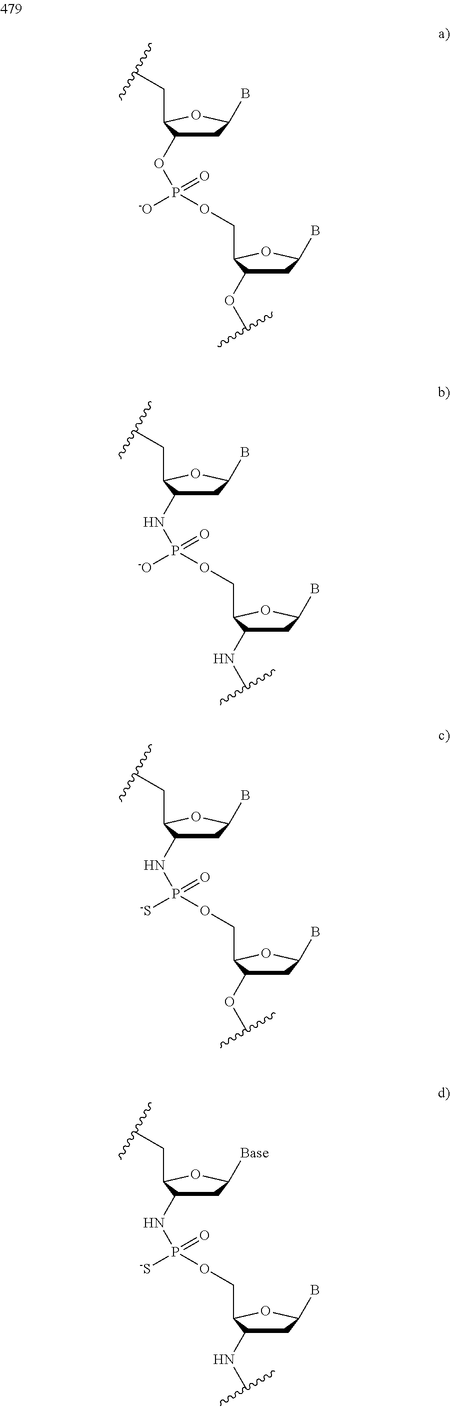

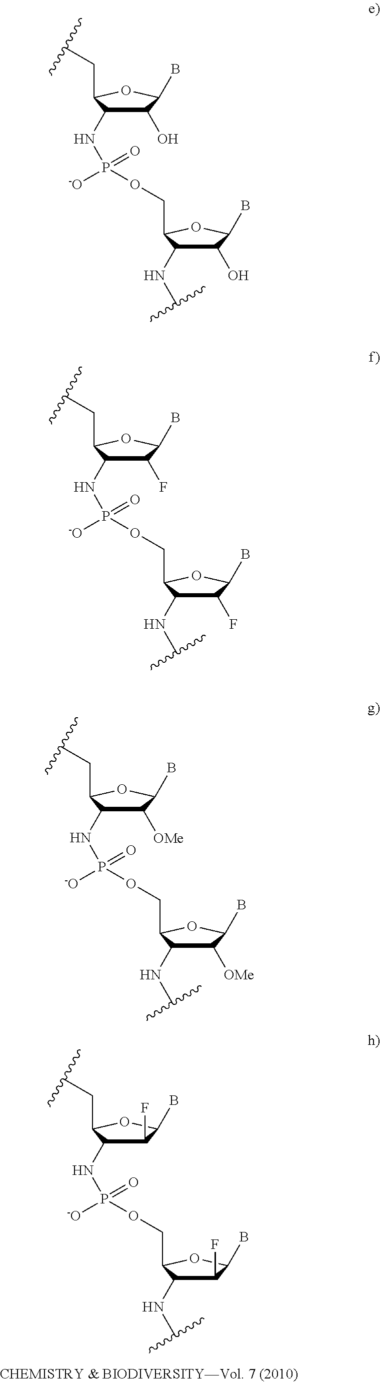

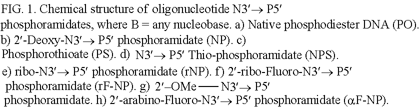

Target polynucleotide: A target polynucleotide according to the invention is a nucleic acid which may be made of naturally occurring nucleotides or which may be made of nucleotides which are not known to occur naturally or any mixture thereof, such as DNA, RNA, LNA or PNA, which is intended captured in the method according to the invention.

Probe: A probe is a defined nucleic acid which may be made of naturally occurring nucleotides or which may be made of nucleotides which are not known to occur naturally or any mixture thereof (including but not limited to DNA, RNA, LNA, PNA) that can be used to identify specific target polynucleotide which may be made of naturally occurring nucleotides or which may be made of nucleotides which are not known to occur naturally or any mixture thereof, such as DNA or RNA molecules, bearing the complementary sequence. In one preferred embodiment a probe is defined as a single-stranded DNA, RNA, LNA, PNA molecule used for detection of the presence of a complementary sequence among a mixture of other singled-stranded polynucleotide which may be made of naturally occurring nucleotides or which may be made of nucleotides which are not known to occur naturally or any mixture thereof, such as e.g. DNA and/or RNA molecules.

Abasic site: Loss of a base in a polynucleotide which may be made of naturally occurring nucleotides or which may be made of nucleotides which are not known to occur naturally or any mixture thereof, such as e.g. in DNA or RNA results in creation of an abasic site leaving a nucleoside such as a deoxyribose residue in the strand. Loss of a base in polynucleotide which may be made of naturally occurring nucleotides or which may be made of nucleotides which are not known to occur naturally or any mixture thereof, such as e.g. DNA or RNA, is a frequent lesion that may occur spontaneously, or under the action of radiations and alkylating agents, or enzymatically.

Intercalator: A type of molecule that, like a conventional nucleotide, can be inserted in the backbone-structure of a polynucleotide probe and which fit morphologically into an abasic site of a complementary polynucleotide target sequence which may be made of naturally occurring nucleotides or which may be made of nucleotides which are not known to occur naturally or any mixture thereof, such as a DNA probe, a RNA probe, a PNA probe or a LNA probe. The intercalator is thus capable of replacing a nucleobase at its position in the probe. The intercalator can be inserted into an abasic site of a complementary polynucleotide which may be made of naturally occurring nucleotides or which may be made of nucleotides which are not known to occur naturally or any mixture thereof, such as e.g. a DNA or RNA structure. This insertion can result in increased stability of the polynucleotide duplex structure. Intercalation occurs when ligands of an appropriate size and chemical nature fit themselves into the abasic site. In the present application the terms intercalator and intercalator molecules are used as synonyms. The intercalator can be as outlined here or it can be any unit which can be inserted into the backbone-structure of the polynucleotide probe and which at the same time is capable of morphologically filling in the abasic site of the target-nucleotide.

TINA: Twisted Intercalating Nucleic Acid. The structures of TINA, para-TINA and ortho-TINA are illustrated in FIG. 5.

INA: The structure of INA is illustrated in FIG. 5.

AMANY: The structure of AMANY is illustrated in FIG. 5.

DNA: The term DNA (Deoxyribonucleic acid) duplex as used herein is a polymer of simple units called nucleotides, with a backbone made of sugars and phosphate atoms joined by ester bonds. A base is attached to each sugar. The bases can be either C, G, T, U or A.

Label: Label herein is used interchangeable with labeling molecule. Label as described herein is an identifiable substance that is detectable in an assay and that can be attached to a molecule creating a labeled molecule.

Alkyl: The term ‘alkyl’ refers to a C₁₋₆-alk(en/yn)yl, a C₃₋₈-cycloalk(en)yl or a C₃₋₈-cycloalk(en)yl-C₁₋₆-alk(en/yn)yl group. The term ‘C₁₋₆-alk(en/yn)yl’ refers to a C₁₋₆-alkyl, a C₂₋₆-alkenyl or a C₂₋₆-alkynyl group, wherein ‘C₁₋₆ alkyl’ refers to a branched or unbranched alkyl group having from one to six carbon atoms inclusive, such as methyl, ethyl, 1-propyl, 2-propyl, 1-butyl, 2-butyl, 2-methyl-2-propyl and 2-methyl-1-propyl; and ‘C₂₋₆ alkenyl’ refers to groups having from two to six carbon atoms, including at least one double bond, such as ethenyl, propenyl, and butenyl; and ‘C₂₋₆ alkynyl’ refers to groups having from two to six carbon atoms, including one triple bond, such as ethynyl, propynyl and butynyl. The term ‘C₃₋₈-cycloalk(en)yl’ refers to a C₃₋₈-cycloalkyl or a C₃₋₈-cycloalkenyl group, wherein ‘C₃₋₈-cycloalkyl’ designates a monocyclic or bicyclic carbocycle having three to eight carbon atoms, such as cyclopropyl, cyclopentyl and cyclohexyl; and ‘C₃₋₈-cycloalkenyl’ refers to a monocyclic or bicyclic carbocycle having three to eight C-atoms and one double bond, such as cyclopropenyl, cyclopentenyl, cyclohexenyl. In the term ‘C₃₋₈-cycloalk(en)yl-C₁₋₆-alk(en/yn)yl’, the terms “C₃₋₈-cycloalk(en)yl” and “C₁₋₅-alk(en/yn)yl” are as defined above.

Heteroatom: An atom selected from the group consisting of nitrogen (N), sulphur (S), oxygen (O), chloro (Cl), bromo (Br), Iodo (I), and fluoro (F).

Aryl: A carbocyclic aromatic group, which is preferably mono- or bicyclic, e.g. phenyl or naphthyl. Thus, the aryl is optionally substituted with one or more substituents, e.g., C₁₋₆-alkyl or halogen.

Heteroaryl: An aromatic group containing at least one carbon atom and one or more heteroatoms selected from O or N or combinations of O and N wherein said aromatic group is preferably mono- or bicyclic.

Polyaromate: A carbocyclic aromatic group comprising at least 2 aromatic groups.

Heteropolyaromate: An aromatic group containing at least one carbon atom and one or more heteroatoms selected from O, N or S or combinations of O and N,N and S pr S and O wherein said aromatic group comprises at least 2 aromatic groups.

### FIGURE LEGENDS

FIG. 1: One type of the bases is removed from double stranded target DNA. This results in a destabilized double stranded target DNA which is subsequently denatured into single stranded target DNA. The single stranded target DNA is mixed with a complementary probe comprising one or more intercalators such as ortho-TINA. This results in capture of the target DNA by the complementary probe.

FIG. 2: One type of the bases is removed from double stranded target DNA. This results in a destabilized double stranded target DNA which is subsequently denatured into single stranded target DNA. The single stranded target DNA is mixed with a complementary probe comprising one or more intercalators such as ortho-TINA. The complementary probe is connected to a support such as a bead. This results in capture of the target DNA by the complementary probe. Subsequently, a detection probe comprising a label is added. In one embodiment one or more washing step(s) are conducted prior to addition of the detection probe.

FIG. 3: One type of the bases in the double stranded target DNA is converted to another chemical entity such as uracil. Subsequently, the chemical entity such as uracil is removed from the double stranded target DNA. This results in a destabilized double stranded target DNA which is subsequently denatured into single stranded target DNA. The single stranded target DNA is mixed with a complementary probe comprising one or more intercalators such as ortho-TINA. This results in capture of the target DNA by the complementary probe.

FIG. 4: One type of the bases in the double stranded target DNA is converted to another chemical entity such as uracil. Subsequently, the chemical entity such as uracil is removed from the double stranded target DNA. This results in a destabilized double stranded target DNA which is subsequently denatured into single stranded target DNA. The single stranded target DNA is mixed with a complementary probe comprising one or more intercalators such as ortho-TINA. The complementary probe is connected to a support such as a bead. This results in capture of the target DNA by the complementary probe. Subsequently, a detection probe comprising a label is added. In one embodiment one or more washing step(s) are conducted prior to addition of the detection probe.

FIG. 5: The chemical structure of TINA, INA, Para-TINA, ortho-TINA and AMANY is illustrated.

FIG. 6: double stranded target DNA (dsDNA) is treated with bisulphite in order to convert cytosine residues to uracil residues. Uracil residues are subsequently removed by uracil-DNA glycosylase (UNG) in order to generate one or more abasic sites. The dsDNA is converted into single stranded target DNA which is captured by an oligonucleotide comprising TINA. The capture oligonucleotide is coupled to a magnetic bead. In addition, a biotinylated TINA detector oligonucleotide is hybridised to the single stranded target DNA. The capture oligonucleotide and/or the detector oligonucleotide can comprise one or more intercalator molecules inserted into the backbone structure of a polynucleotide probe to morphologically fit into an abasic site of a complementary polynucleotide target sequence wherein said insertion is made after hybridisation with the single stranded target DNA. Alternatively, the capture oligonucleotide and/or the detector oligonucleotide can comprise one or more adenine residues which can be inserted at the one or more abasic sites after hybridisation with the single stranded target DNA. Under the reaction conditions used for the experiment illustrated in FIG. 6 the detector oligonucleotides and capture oligonucleotides with intercalator molecules will preferably hybridise to the single stranded target DNA. Streptavidin-R-phycoerythrin is used for the detection.

FIG. 7: double stranded target DNA (dsDNA) is treated with bisulphite in order to convert cytosine residues to uracil residues. Uracil residues are subsequently removed by uracil-DNA glycosylase (UNG) in order to generate one or more abasic sites. The dsDNA is converted into single stranded target DNA which is captured by an oligonucleotide comprising TINA. The capture oligonucleotide is coupled to a magnetic bead. In addition, a biotinylated TINA detector oligonucleotide is hybridised to the single stranded target DNA. The capture oligonucleotide and/or the detector oligonucleotide can comprise one or more intercalator molecules inserted into the backbone structure of a polynucleotide probe and fitting morphologically into an abasic site of a complementary polynucleotide target sequence after hybridisation with the single stranded target DNA. Alternatively, the capture oligonucleotide and/or the detector oligonucleotide can comprise one or more adenine residues which can be inserted at the one or more abasic sites after hybridisation with the single stranded target DNA. Under the reaction conditions used for the experiment illustrated in FIG. 7 the detector oligonucleotides and capture oligonucleotides with either intercalator molecules or with adenine residues will hybridise to the single stranded target DNA. Streptavidin-R-phycoerythrin is used for the detection.

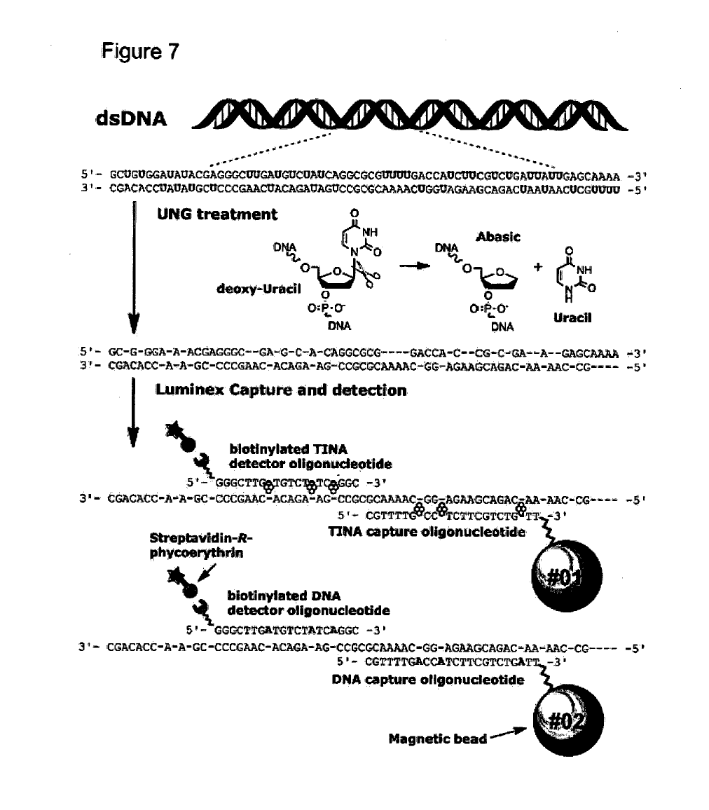

FIG. 8: Detection of uracil modified dsDNA after uracil-DNA glycosylase treatment. The x-axis shows the amount of dsDNA target (mol) and the y-axis shows MFI-zero.

### DETAILED DESCRIPTION OF THE INVENTION

The present invention relates to a technology which can e.g. be used for capture of polynucleotide such as single stranded target polynucleotide which may be made of naturally occurring nucleotides or which may be made of a nucleotides which are not known to occur naturally or any mixture thereof, such as e.g. DNA and/or RNA. This technique can e.g. be used for molecular diagnostics. Accordingly, one embodiment relates to a method for detection of one or more methylated target DNA and/or RNA comprising use of the method disclosed herein.

The method disclosed herein for use in detection of target polynucleotide is more specific than the methods known in the art. Due to a significant deviation in Tm, it is possible to rutineously separate target polynucleotide from other nucleotide(s) even when said target polynucleotide and said other nucleotide(s) differ in only a single base position.

Even though the aim is to improve the stability of the target polynucleotide, the initial stage is to destabilize it. This is highly controversial and has not previously been described. Accordingly, the method disclosed herein involves cleavage of target polynucleotide which leads to the creation of a modified polynucleotide having improved properties in respect of stability.

Accordingly, the present invention relates to a method for capture of target polynucleotide such as single stranded target polynucleotide, such as e.g. DNA or RNA, from a sample comprising the steps of:
iii) removal of one or more of the types of bases A, T, U, C or G, 5-hydroxymethyl-dC, 5-methylcytosine (m⁵C), pseudouridine (Ψ), dihydrouridine (D), inosine (I), 7-methylguanosine (m⁷G), hypoxanthine, xanthine and their 2′-O-Methyl-derivatives and/or N-Methyl-derivatives from said target polynucleotide thereby generating one or more abasic sites and
iv) capture of said target polynucleotide with a complementary probe comprising one or more intercalator molecules inserted into the backbone-structure of a polynucleotide probe fitting morphologically into one or more abasic sites of a complementary polynucleotide target sequence
   wherein said target polynucleotide may be made of naturally occurring nucleotides or of a nucleotides which are not known to occur naturally or any mixture thereof, saod target polynucleotide may thus e.g. be made of nucleotides such as those selected from the group consisting of RNA, α-L-RNA, β-D-RNA, 2′-R-RNA, DNA, LNA, PNA, PMO, TNA, GNA, oligonucleotide N3′→P5′ phosphoramidates, BNA, α-L-LNA, HNA, MNA, ANA, CAN, INA, CeNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-OR-RNA, 2′-AE-RNA, and combinations and modifications thereof.

In a preferred embodiment the present invention relates to a method for capture of single stranded target polynucleotide comprising the steps of:
(v) providing double stranded target polynucleotide which may be made of naturally occurring nucleotides or which may be made of nucleotides which are not known to occur naturally or any mixture thereof, such as e.g. DNA;
(vi) destabilisation of said double stranded target polynucleotide, such as e.g. DNA, by removal of one or more of the types of bases from said double stranded target polynucleotide, such as e.g. DNA, thereby generating one or more abasic sites;
(vii) denaturing of said destabilized double stranded target polynucleotide, such as e.g. DNA, to single stranded target polynucleotide, such as e.g. DNA, and
(viii) capture of said single stranded target polynucleotide, such as e.g. DNA, with a complementary polynucleotide probe, such as e.g. a DNA probe, comprising one or more intercalator molecules inserted into the backbone-structure of a polynucleotide probe fitting morphologically into one or more abasic sites of a complementary polynucleotide target sequence.

In a specific embodiment, the present invention relates to a method for capture of polynucleotide such as single stranded target DNA or RNA from a sample which comprise the following steps
(i) removal of one or more of the types of bases A, T, U, C or G from said target polynucleotide such as DNA or RNA thereby generating one or more abasic sites and
(ii) capture of said target polynucleotide such as DNA or RNA with a complementary probe comprising one or more intercalator molecules which can interact with and/or be inserted in the one ore more abasic sites.

The optimal length of the target polynucleotide according to the present invention depends upon the number of specific bases to be removed and also of their distribution in said target polynucleotide. Generally, the length of such target polynucleotide is preferably less than 30 bases. In a preferred embodiment thereof, the length of said target polynucleotide is less than 25 bases. In an even more preferred embodiment thereof, the length of said target polynucleotide is less than 20 bases, such as 17-18 bases.

The abasic sites comprised in single stranded target polynucleotide from which one or more types of bases has been removed, are preferably placed not too close to each other, such as e.g. with a distance of ½ or 1 helix turn. However, removal of 2 adjacent bases would also be possible. In the latter case, the intercalator inserted would be one double-sized intercalator or 2 conventional intercalators. Accordingly, one embodiment is directed to such targets wherein the abasic sites are placed with a distance of ½ or 1 helix turn, such as 1 helix turn, such as ½ helix turn. Accordingly, one embodiment is directed to such targets wherein the abasic sites are placed with a distance of ½ or 1 helix turn, such as 1 helix turn, such as ½ helix turn. In a preferred embodiment, one intercalator is inserted in each abasic site. In one embodiment thereof, more than abasic site is present and the intercalators inserted are identical. In another embodiment thereof, more than one abasic site is present and the intercalators inserted are different from each other.

In a preferred embodiment of the invention, the total number of abasic sites present in said target polynucleotide does not exceed 5. In a more preferred embodiment thereof, the total number of abasic sites is at most 4. In a most preferred embodiment of the invention, said target polynucleotide comprises no more than 3 abasic sites, such as e.g. 2 abasic sites. In a further embodiment of the invention, said target polynucleotide comprises 1 abasic site.

By the removal of one or more of a specific type of base as defined herein, e.g. by removal of one or more of the following type(s) of bases A, T, U, C or G, from said target polynucleotide in above step (i), at least 70%, for example at least 80%, such as at least 85%, for example at least 90%, such as at least 95%, for example at least 97%, such as at least 99% of the base concerned is removed from the target polynucleotide. In a preferred embodiment about 100% of the base concerned is removed from the target polynucleotide.

In one particular embodiment the target polynucleotide may be made of naturally occurring nucleotides or of nucleotides which are not known to occur naturally or any mixture thereof, such as RNA or DNA. In one specific embodiment thereof, said target polynucleotide is made of naturally occurring nucleotides. In one specific embodiment thereof, said target polynucleotide comprises RNA. In another specific embodiment thereof, said target polynucleotide comprises DNA. In another particular embodiment, the target polynucleotide is made of nucleotides which are not known to occur naturally or any mixture of naturally occurring nucleotides and nucleotides not known to occur naturally. Said target polynucleotide thus comprises one or more analogue(s) of the naturally occurring nucleotides which e.g. may be selected from the group consisting of LNA, PNA, PMO, TNA, GNA, nucleotide N3′→P5′ phosphoramidates, BNA, α-L-LNA and other constrained nucleotides. In one specific embodiment thereof said target polynucleotide comprises LNA. In one specific embodiment thereof said target polynucleotide comprises PNA. In one specific embodiment thereof said target polynucleotide comprises PMO. In one specific embodiment thereof said target polynucleotide comprises TNA. In one specific embodiment thereof said target polynucleotide comprises GNA. In one specific embodiment thereof said target polynucleotide comprises nucleotide N3′→P5′ phosphoramidates. In one specific embodiment thereof said target polynucleotide comprises BNA. In one specific embodiment thereof said target polynucleotide comprises α-L-LNA. In one specific embodiment thereof said target polynucleotide comprises other constrained nucleotides as defined herein.

In one embodiment, the base removed is selected from the group consisting of A, G, C, T, U and 5-hydroxymethyl-dC.
In an embodiment the base removed is A from the double stranded target polynucleotide and/or single stranded polynucleotide.

In one specific embodiment thereof, A is removed from the target polynucleotide by submitting said target polynucleotide to enzymatic treatment, e.g. ANG treatment which designates treatment with Adenine-DNA glycosylase.

In another embodiment the base removed is T from the double stranded target polynucleotide and/or single stranded polynucleotide. In one specific embodiment thereof, T is removed from the target polynucleotide by submitting said target polynucleotide to enzymatic treatment, e.g. TNG treatment which designates treatment with Thymine-DNA glycosylase.

In another embodiment the base removed is U from the double stranded target polynucleotide and/or single stranded polynucleotide. In one specific embodiment thereof, U is removed from the target polynucleotide by submitting said target polynucleotide to enzymatic treatment, e.g. UNG treatment which designates treatment with Uracil-DNA glycosylase.

In another embodiment the base removed is C from the double stranded target polynucleotide and/or single stranded polynucleotide. In one specific embodiment thereof, C is removed from the target polynucleotide by submitting said target polynucleotide to enzymatic treatment, e.g. CNG treatment which designates treatment with Cytosine-DNA glycosylase.

In another embodiment the base removed is G from the double stranded target polynucleotide and/or single stranded polynucleotide. In one specific embodiment thereof, G is removed from the target polynucleotide by submitting said target polynucleotide to enzymatic treatment, e.g. GNG treatment which designates treatment with Guanine-DNA glycosylase.

In another embodiment the base removed is 5-hydroxymethyl-dC.

In another embodiment the base removed is 5-methylcytosine (m⁵C).

In another embodiment the base removed is pseudouridine (Ψ).

In another embodiment the base removed is dihydrouridine (D).

In another embodiment the base removed is inosine (I).

In another embodiment the base removed is 7-methylguanosine (m⁷G).

In another embodiment the base removed is hypoxanthine.

In another embodiment the base removed is xanthine.

In another embodiment the base removed is a 2′-O-Methyl-derivative of any one of the bases disclosed herein.

In another embodiment the base removed is a N-Methyl-derivative of any one of the bases disclosed herein.

The complementary probe can be any polynucleotide probe such as a probe which may be made of a naturally occurring nucleotides or which may be made of nucleotides which are not known to occur naturally or any mixture thereof, such as e.g. a probe selected from the group consisting of a DNA probe, a RNA probe, a LNA probe and a PNA probe or any combinations thereof.

The complementary probe can be any polynucleotide probe which may be made of a naturally occurring nucleotides or which may be made of nucleotides which are not known to occur naturally or any mixture thereof, such as a DNA probe, a RNA probe, a LNA probe or PNA probe or any combinations thereof. In one particular embodiment the complementary probe is made of naturally occurring nucleotides, such as RNA or DNA. In one specific embodiment thereof, said complementary probe comprises RNA. In another specific embodiment thereof, said complementary probe is comprises DNA. In another particular embodiment, the complementary probe is made of a polynucleotide sequence which is not known to occur naturally. In a specific embodiment thereof, said complementary probe comprises one or more analogue(s) selected from the group consisting of LNA, PNA, PMO, TNA, GNA, oligonucleotide N3′→P5′ phosphoramidates, BNA, α-L-LNA and other constrained nucleotides. In one specific embodiment thereof said complementary probe comprises LNA. In one specific embodiment thereof said complementary probe comprises PNA. In one specific embodiment thereof said complementary probe comprises PMO. In one specific embodiment thereof said complementary probe comprises TNA. In one specific embodiment thereof said complementary probe comprises GNA. In one specific embodiment thereof said complementary probe comprises oligonucleotide N3′→P5′ phosphoramidates. In one specific embodiment thereof said complementary probe comprises BNA. In one specific embodiment thereof said complementary probe comprises α-L-LNA. In one specific embodiment thereof said complementary probe comprises other constrained nucleotides as defined herein.

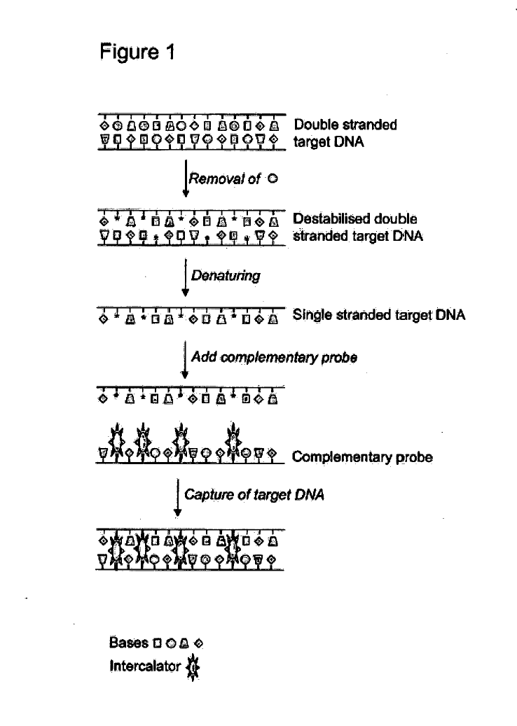

The technique comprises specific capture of polynucleotide which may be made of a naturally occurring nucleotides or which may be made of nucleotides which are not known to occur naturally or any mixture thereof, such as single stranded target DNA and/or RNA by a complementary probe comprising one or more intercalator molecules. The method further involves removal of one or more type(s) of bases from the target polynucleotide which may be made of a naturally occurring nucleotides or which may be made of nucleotides which are not known to occur naturally or any mixture thereof, such as DNA and/or RNA, prior to interaction with the complementary probe. This reaction results in generation of one or more abasic sites—i.e. a site where the base has been removed. The removal of the bases from the target polynucleotide which may be made of a naturally occurring nucleotides or which may be made of nucleotides which are not known to occur naturally or any mixture thereof, such as DNA and/or RNA, can either be removed from the double stranded target polynucleotide such as DNA or from the single stranded target polynucleotide such as DNA and/or RNA. In one embodiment the bases are preferably removed from double stranded target DNA. This embodiment is exemplified in FIG. 1.

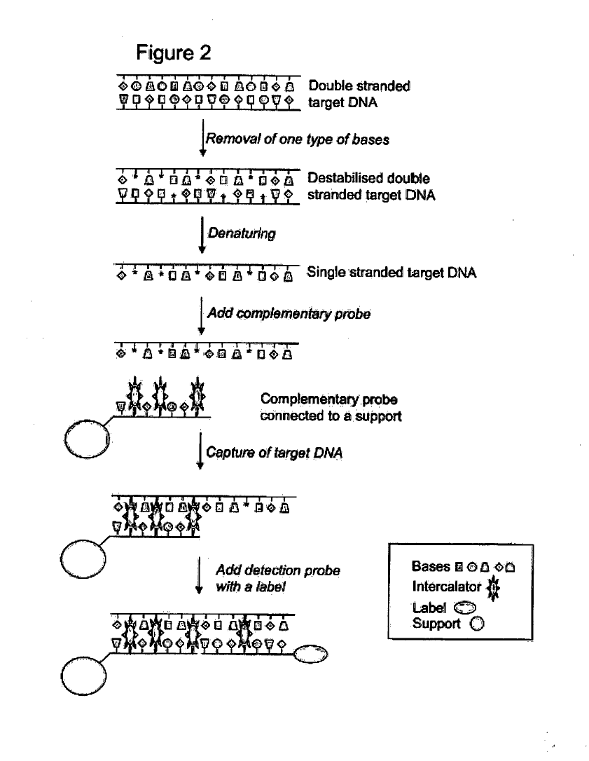

The complementary probe can be connected to a support such as a bead. This results in capture of the target polynucleotide which may be made of a naturally occurring nucleotides or which may be made of nucleotides which are not known to occur naturally or any mixture thereof, such as DNA and/or RNA, by the complementary probe. Subsequently, a detection probe comprising one or more labels can be added. In one embodiment, the method disclosed herein further comprises one or more washing steps in order to remove of unbound polynucleotides and nucleotides which may be made of naturally occurring nucleotides or which may be made of nucleotides which are not known to occur in nature or any mixture thereof. In one embodiment said washing step is conducted prior to addition of the detection probe e.g. to remove unspecific polynucleotides and nucleotides which may be made of a naturally occurring nucleotides or which may be made of nucleotides which are not known to occur naturally or any mixture thereof, such as e.g. DNA and/or RNA. This embodiment is exemplified in FIG. 2.

The removal of the bases from the target polynucleotide which may be made of a naturally occurring nucleotides or which may be made of nucleotides which are not known to occur naturally or any mixture thereof, such as e.g. such as DNA and/or RNA, can e.g. be performed by use of an enzyme that specifically removes any base disclosed herein such as e.g. A, T, C, G and/or U. Alternatively one type of bases can be removed by use of reaction conditions that specifically results in loss of one of the types of bases. A can for example be removed by regulation of the pH [7, 8, 9]. In a particular embodiment, 1, 2 or 3 types of the bases from the target polynucleotide such as target DNA and/or RNA, is removed.

In an embodiment, the method disclosed herein comprises destabilisation of double stranded target polynucleotide which may be made of nucleotides which are not known to occur naturally or any mixture thereof by removal of one or more chemical entities from said double stranded target polynucleotide.

In one embodiment one type of the bases in the double stranded target polynucleotide which may be made of a naturally occurring nucleotides or which may be made of nucleotides which are not known to occur naturally or any mixture thereof, such as DNA and/or single stranded DNA and/or RNA is converted into another chemical entity such as uracil. Subsequently, the chemical entity such as uracil is removed from the double stranded target polynucleotide which may be made of a naturally occurring nucleotides or which may be made of nucleotides which are not known to occur naturally or any mixture thereof, such as DNA, and/or from the single stranded target polynucleotide which may be made of a naturally occurring nucleotides or which may be made of nucleotides which are not known to occur naturally or any mixture thereof, such as DNA and/or RNA. In an embodiment, the removal of the chemical entity is performed by use of one or more enzymes and/or by physical stress such as change of the salt concentration, pH [7, 8, 9] or temperature etc. MutY is an adenine glycosylase which is active on G-A mispairs. MutY can in one embodiment be used for removal of bases in the DNA [8].

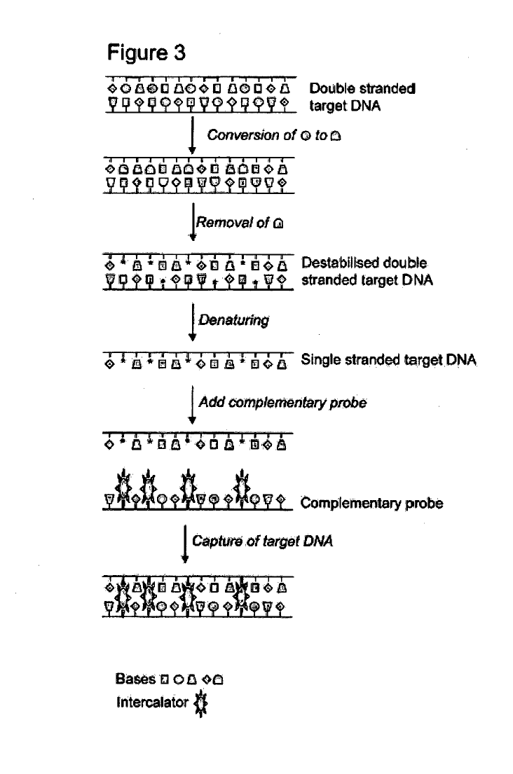

In another embodiment excision of cytosine and thymine from polynucleotide which may be made of a naturally occurring nucleotides or which may be made of nucleotides which are not known to occur naturally or any mixture thereof, such as e.g. DNA or RNA, can be performed by mutants of human uracil-DNA glycosylase [9]. In one preferred embodiment replacement of Asn204 in Uracil-DNA glycosylase by Asp or Tyr147 in Uracil-DNA glycosylase by Ala, Cys or Ser result in enzymes that have cytosine-DNA glycosylase activity or thymine-DNA glycosylase activity, respectively [9]. These enzymes can be used for removal of these bases in the polynucleotide which may be made of a naturally occurring nucleotides or which may be made of nucleotides which are not known to occur naturally or any mixture thereof, such as e.g. DNA or RNA. In an embodiment, Uracil is removed by uracil dehydrogenase. In a further embodiment, removel of A is performed by adjustment of the pH value. Removal of the chemical entity from double stranded target polynucleotide which may be made of a naturally occurring nucleotides or which may be made of nucleotides which are not known to occur naturally or any mixture thereof, such as DNA, results in a destabilized double stranded target polynucleotide which may be made of a naturally occurring nucleotides or which may be made of nucleotides which are not known to occur naturally or any mixture thereof, such as DNA, which is subsequently denatured into single stranded target polynucleotide which may be made of a naturally occurring nucleotides or which may be made of nucleotides which are not known to occur naturally or any mixture thereof, such as DNA. The single stranded target polynucleotide which may be made of a naturally occurring nucleotides or which may be made of nucleotides which are not known to occur naturally or any mixture thereof, such as DNA and/or RNA, is mixed with a complementary probe comprising one or more intercalators such as ortho-TINA. This results in capture of the target polynucleotide which may be made of a naturally occurring nucleotides or which may be made of nucleotides which are not known to occur naturally or any mixture thereof, such as DNA by the complementary probe. This embodiment is exemplified in FIG. 3.

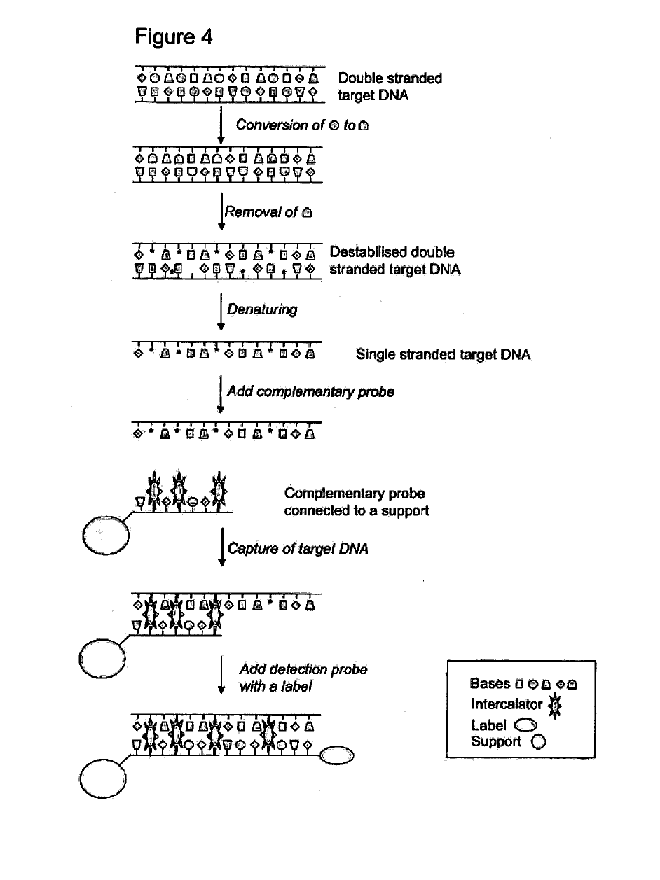

In one embodiment one type of the bases in the double stranded target polynucleotide which may be made of a naturally occurring nucleotides or which may be made of nucleotides which are not known to occur naturally or any mixture thereof, such as DNA and/or single stranded polynucleotide which may be made of a naturally occurring nucleotides or which may be made of nucleotides which are not known to occur naturally or any mixture thereof, such as DNA and/or RNA is converted into another chemical entity such as uracil. Uracil can for example be removed by uracil dehydrogenase. Subsequently, the chemical entity such as uracil is removed from the double stranded target polynucleotide which may be made of a naturally occurring nucleotides or which may be made of nucleotides which are not known to occur naturally or any mixture thereof, such as DNA and/or single stranded target polynucleotide such as DNA and/or RNA. The removal of the chemical entity can be performed by use of one or more enzymes and/or by physical stress such as change of salt concentration, pH etc. [7, 8, 9]. Uracil can for example be removed by uracil dehydrogenase. Removal of the chemical entity from double stranded polynucleotide which may be made of a naturally occurring nucleotides or which may be made of nucleotides which are not known to occur naturally or any mixture thereof, such as DNA results, in a destabilized double stranded target polynucleotide, such as DNA, which is subsequently denatured into single stranded target polynucleotide, such as DNA. The single stranded target polynucleotide which may be made of a naturally occurring nucleotides or which may be made of nucleotides which are not known to occur naturally or any mixture thereof, such as DNA and/or RNA is mixed with a complementary probe comprising one or more intercalators such as ortho-TINA. The complementary probe is connected to a support such as a bead. This results in capture of the target polynucleotide which may be made of a naturally occurring nucleotides or which may be made of nucleotides which are not known to occur naturally or any mixture thereof, such as DNA by the complementary probe. Subsequently, a detection probe comprising a label is added. In one embodiment a washing step is conducted prior to addition of the detection probe e.g. to remove unspecific polynucleotide and nucleotide which may be made of a naturally occurring nucleotides or which may be made of nucleotides which are not known to occur naturally or any mixture thereof, such as e.g. DNA and/or RNA. This embodiment is illustrated in FIG. 4.

The invention comprises removal of 1, 2, or 3 types of the bases from the target polynucleotide which may be made of a naturally occurring nucleotides or which may be made of nucleotides which are not known to occur naturally or any mixture thereof, such as DNA and/or RNA. Accordingly, in a first embodiment a base as disclosed herein is removed, such as either A, T, C, G or U is removed. In a further embodiment 2 or 3 types of the bases are removed such as removal of A and T.

Bases from both the target polynucleotide and other nucleotide material within the test material are removed when using the method of the present invention. Accordingly, the bases removed from the target polynucleotide which may be made of a naturally occurring nucleotides or which may be made of nucleotides which are not known to occur naturally or any mixture thereof, such as DNA and/or RNA only constitutes a minor part of the nucleotide material present in the test material.

One embodiment of the invention relates to the method disclosed herein, wherein the total number of bases that are removed from the target polynucleotide, such as RNA or DNA, can be selected from the group consisting of 1, 2, 3, 4, 5, 6, 7, 8, 9, 10, 11, 12, 13, 14, 15, 16, 17, 18, 19, 20 and more than 20 bases.

In one specific embodiment, the total number of uracil residues or other chemical entities that are removed from the target polynucleotide which may be made of a naturally occurring nucleotides or which may be made of nucleotides which are not known to occur naturally or any mixture thereof, such as e.g. DNA and/or RNA can be any number such as 0, 1, 2, 3, 4, 5, 6, 7, 8, 9, 10, 11, 12, 13, 14, 15, 16, 17, 18, 19, 20 or more than 20 different or identical chemical entities or uracil residues.

In another embodiment the total number of uracil residues or other chemical entities that are removed from the target which may be made of a naturally occurring nucleotides or which may be made of nucleotides which are not known to occur naturally or any mixture thereof, such as e.g. DNA and/or RNA is at least 1, such as at least 2, for example 3, such as at least 4, for example 5, such as at least 6, for example 7, such as at least 8, for example 9, such as at least 10, for example 11, such as at least 12, for example 13, such as at least 14, for example 15, such as at least 16, for example 17, such as at least 18, for example 19, or such as at least 20.

In another embodiment the total number of uracil residues or other chemical entities that are removed from the target polynucleotide which may be made of a naturally occurring nucleotides or which may be made of nucleotides which are not known to occur naturally or any mixture thereof, such as e.g. DNA and/or RNA is less than 20, such as less than 19, for example less than 18, such as less than 17, for example less than 16, such as less than 15, for example less than 14, such as less than 13, for example less than 12, such as less than 11, for example less than 10, such as less than 9, for example less than 8, such as less than 7, for example less than 6, such as less than 5, for example less than 4, such as less than 3, for example less than 2, or such as less than 1.

The number of residues that are removed correlates with the change of the melting temperature of dsDNA. In one embodiment the preferred change of the melting temperature corresponds to removal of less than 5 uracil residues or other chemical entities. In other embodiments the preferred change in melting temperature corresponds to removal of from 5 to 10 uracil residues or other chemical entities. Finally, in another preferred embodiments the preferred change in melting temperature corresponds to removal of at least 10 uracil residues or other chemical entities.

The total number of intercalator molecules in the complementary probe can be selected from the group consisting of 1, 2, 3, 4, 5, 6, 7, 8, 9, 10, 11, 12, 13, 14, 15, 16, 17, 18, 19, 20 and more than 20 different or identical intercalation molecules. Accordingly, one embodiment relates to a polynucleotide probe which is suitable for interaction with a nucleotide target, such as a complementary RNA and/or DNA target, wherein said polynucleotide probe comprises exactly 1 intercalator molecule. Another embodiment relates to a polynucleotide probe suitable for interaction with a complementary nucleotide target, such as a complementary RNA and/or DNA target, wherein the polynucleotide probe comprises at least 2 intercalator molecules. A further embodiment relates to a polynucleotide probe which may be made of naturally occurring nucleotides or which may be made of nucleotides which are not known to occur in nature or any mixture thereof said complementary probe may thus e.g. be made of nucleotides such as those selected from the group consisting of RNA, α-L-RNA, β-D-RNA, 2′-R-RNA, DNA, LNA, PNA, PMO, TNA, GNA, oligonucleotide N3′→P5′ phosphoramidates, BNA, α-L-LNA, HNA, MNA, ANA, CAN, INA, CeNA, (2′-NH)-TNA, (3′-NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-OR-RNA, 2′-AE-RNA, and combinations and modifications thereof. One particular embodiment thereof relates to such probe which is selected from the group consisting of a DNA probe, a RNA probe, a LNA probe and a PNA probe. One further embodiment relates to a polynucleotide probe wherein the total number of intercalator molecules can be selected from the group consisting of 2, 3, 4, 5, 6, 7, 8, 9, 10, 11, 12, 13, 14, 15, 16, 17, 18, 19, 20 and more than 20 intercalator molecules. In further specific embodiments thereof the total number of intercalator molecules in the complementary probe is at least 1, such as at least 2, for example 3, such as at least 4, for example 5, such as at least 6, for example 7, such as at least 8, for example 9, such as at least 10, for example 11, such as at least 12, for example 13, such as at least 14, for example 15, such as at least 16, for example 17, such as at least 18, for example 19, or such as at least 20.

In another embodiment the total number of intercalator molecules in the complementary probe is less than 20, such as less than 19, for example less than 18, such as less than 17, for example less than 16, such as less than 15, for example less than 14, such as less than 13, for example less than 12, such as less than 11, for example less than 10, such as less than 9, for example less than 8, such as less than 7, for example less than 6, such as less than 5, for example less than 4, such as less than 3, for example less than 2, or such as less than 1.

The number of intercalator molecules in the complementary probe which may be made of a naturally occurring nucleotides or which may be made of nucleotides which are not known to occur naturally or any mixture thereof correlates with a change of the melting temperature of the hybridization product consisting of the target DNA or RNA and the complementary probe. In one embodiment the preferred change of the melting temperature corresponds to use of a probe with less than 5 intercalator molecules. In other embodiments the preferred change in melting temperature corresponds to use of a probe with from 5 to 10 intercalator probes. Finally, in another preferred embodiments the preferred change in melting temperature corresponds to use of a probe with at least 10 intercalator molecules.

In a preferred embodiment, one intercalator molecule is inserted into the backbone-structure of a polynucleotide probe fitting morphologically into one abasic site of a complementary polynucleotide target sequence. In one embodiment thereof, more than one abasic site is present and the intercalators inserted therein are identical. In another embodiment thereof, more than one abasic site is present and the intercalators inserted are different from each other.

In a preferred embodiment of the invention, the total number of intercalators inserted into abasic sites is identical to the number of abasic sites present in the target polynucleotide. Particularly preferred is that the number of abasic sites and thus the number of intercalators inserted into abasic sites does not exceed 5. In a more preferred embodiment thereof, the total number of abasic sites and thus the number of intercalators inserted into abasic sites is at most 4. In a most preferred embodiment of the invention, said target polynucleotide comprises no more than 3 abasic sites and thus no more than 3 intercalators are inserted into abasic sites, such as e.g. 2 abasic sites and intercalators inserted into abasic sites. In a further embodiment of the invention, said target polynucleotide comprises 1 abasic site and 1 intercalator is inserted into said abasic site.

As previously mentioned the intercalator molecules in a probe can be either different or identical. In one embodiment different intercalator molecules are used to optimize the hybridization specificity of the probe to the target polynucleotide which may be made of a naturally occurring nucleotides or which may be made of nucleotides which are not known to occur naturally or any mixture thereof, such as e.g. DNA or RNA. For some types of intercalator molecules it is preferred to use different types of intercalator molecules if these intercalator molecules are close to each other in the probe such as next to each other or if there is less than 2, 3, 4, 5, or 6 residues between them. For other types of intercalator molecules it is preferred to use identical types of intercalator molecules if these intercalator molecules are close to each other in the probe such as next to each other or if there is less than 2, 3, 4, 5, or 6 residues between them.

The length of the complementary probe is in one embodiment selected from the group consisting of 5, 6, 7, 8, 9, 10, 11, 12, 13, 14, 15, 16, 17, 18, 19, 20, 21, 22, 23, 24, 25, 26, 27, 28, 29, 30 and more than 30 bases. Preferably, the length of the complementary probe is from 18 to 22 bases long. The length of the probe is optimized in other to optimize the hybridization efficiency. The hybridization efficiency depends on the specific sequence as well as the number of abasic sites.

In one embodiment, the method disclosed herein further comprises conversion of one or more C's in the target polynucleotide which may be made of a naturally occurring nucleotides or which may be made of nucleotides which are not known to occur naturally or any mixture thereof to one or more U's.

In one embodiment, the method disclosed herein further comprises conversion of one or more types of bases in the double stranded target polynucleotide to another chemical entity. In a specific embodiment thereof, the method can further comprise conversion of one or more C's in the double stranded target polynucleotide which may be made of a naturally occurring nucleotides or which may be made of nucleotides which are not known to occur naturally or any mixture thereof, such as e.g. DNA, to one or more U's prior to removal of any bases. The conversion of one or more C's in the double stranded target DNA to one or more U's can be preformed by bisulphite treatment [1].

In one embodiment, in the method disclosed herein the conversion of one or more C's in the target polynucleotide to one or more U's is preformed by bisulphite treatment.

In another embodiment one type of the bases is removed from double stranded target polynucleotide which may be made of a naturally occurring nucleotides or which may be made of nucleotides which are not known to occur naturally or any mixture thereof, such as from DNA and/or from single stranded DNA and/or RNA. The removal of one type of bases from double stranded polynucleotide which may be made of a naturally occurring nucleotides or which may be made of nucleotides which are not known to occur naturally or any mixture thereof, such as DNA results in a destabilized double stranded target polynucleotide, such as DNA, which is subsequently denatured into single stranded target polynucleotide, such as DNA. The single stranded target polynucleotide which may be made of a naturally occurring nucleotides or which may be made of nucleotides which are not known to occur naturally or any mixture thereof, such as DNA and/or RNA is subsequently mixed with a complementary probe comprising one or more intercalators such as ortho-TINA. The complementary probe can be connected to a support such as a bead. This results in capture of the target polynucleotide which may be made of a naturally occurring nucleotides or which may be made of nucleotides which are not known to occur naturally or any mixture thereof, such as DNA and/or RNA, by the complementary probe. Subsequently, a detection probe comprising a label can be added. In one embodiment a washing step is conducted prior to and/or after addition of the detection probe.

In one embodiment, the polynucleotide probe suitable for interaction with a complementary nucleotide target which may be made of a naturally occurring nucleotides or which may be made of nucleotides which are not known to occur naturally or any mixture thereof, such as e.g. a RNA and/or a DNA target, comprises exactly 1 intercalator molecule. In one embodiment, the polynucleotide probe suitable for interaction with a complementary nucleotide target which may be made of a naturally occurring nucleotides or which may be made of nucleotides which are not known to occur naturally or any mixture thereof, such as e.g. a RNA and/or a DNA target comprises at least 2 intercalator molecules.

In one preferred embodiment the complementary probe and the detection probe comprises one or more intercalator molecules. This has the advantage that the complementary probe and the detection probe cannot hybridise to each other.

In an embodiment of the method of the present invention, the complementary probe comprises one or more intercalator molecules. In a particular embodiment thereof, the total number of intercalator molecules can be selected from the group consisting of 1, 2, 3, 4, 5, 6, 7, 8, 9, 10, 11, 12, 13, 14, 15, 16, 17, 18, 19, 20 and more than 20 intercalator molecules.

The method according to present invention relates in one embodiment to a method wherein an intercalator molecule has been inserted into from 10% to 100% of the abasic sites in the target polynucleotide which may be made of a naturally occurring nucleotides or which may be made of nucleotides which are not known to occur naturally or any mixture thereof, such as a DNA target and/or a RNA target, such as from 10% to 20%, for example from 20% to 30%, such as from 30% to 40%, for example from 40% to 50%, such as from 50% to 60%, for example from 60% to 70%, such as from 70% to 80%, for example from 80% to 90%, such as from 90% to 100%, or any combination thereof.

The insertion of the intercalator molecules can result in an increased melting point of the polynucleotide duplex consisting of the target polynucleotide which may be made of a naturally occurring nucleotides or which may be made of nucleotides which are not known to occur naturally or any mixture thereof, such as DNA and/or RNA and the complementary probe. One preferred embodiment thereof, relates to a polynucleotide probe, wherein insertion of intercalator molecules into the backbone-structure of a polynucleotide problem, said intercalator molecules fitting morphologically into one or more abasic sites of a complementary polynucleotide target sequence results in increased melting point of a polynucleotide duplex consisting of the target DNA and/or RNA and the complementary probe.

An advantage of the present invention is that a single mutation in the nucleotide target which may be made of a naturally occurring nucleotides or which may be made of nucleotides which are not known to occur naturally or any mixture thereof, such as e.g. the target DNA, can be detected with substantially higher confidence compared to conventional hybridization technology. Removal of one or more bases from the target (creating one or more “abasic” sites) results in a drop in the melting temperature of e.g. approximately 12° C. per abasic site. The exact drop in melting temperature does however depend on the size of target and also on the number of abasic sites in the target. The interaction between a probe containing intercalator molecule(s) “basepairing” (i.e. filling out the empty space) with the abasic site(s) in the target will compensate for this drop in melting temperature. In a preferred embodiment of the invention, the insertion of the intercalator molecules into the backbone-structure of a polynucleotide probe, said intercalator molecule fitting morphologically into one or more abasic sites of a complementary polynucleotide target sequence results in increased melting temperature of the polynucleotide duplex consisting of the target polynucleotide, such as DNA and/or RNA, and the complementary probe. Accordingly, there will be a relative large difference in melting temperature between a probe which is a perfect match to the target DNA and a probe which has a single mis-match. Accordingly, the present technology can more specifically identify a single mis-match compared to hybridization with conventional probes

It has been found that the presence of abasic sites in a probe decrease Tm substantially more compared to nucleobase mismatches.

The inventors of the present invention have found that central nucleobase mismatches decrease Tm more than nucleobase mismatches which are placed towards the ends of the oligonucleotides. Accordingly, one embodiment of the invention relates to such probes wherein the abasic sites or the nucleobase mismatches are placed in the end of the probe nucleotide sequence. Another embodiment of the invention, relates to such probes wherein the abasic sites or the nucleobase mismatches are placed towards the end of the probe nucleotide sequence.

Decreasing the length of the oligonucleotides in the duplex from 30 down to 18 nucleotides decreases the Tm of the duplex. Accordingly, in one embodiment of the invention, the probe nucleotide sequence has a length of 25-35 nucleotides, such as of about 30 nucleotides. In another embodiment, the probe nucleotide sequence has a length of 15-23 nucleotides. In a preferred embodiment, the probe nucleotide sequence has a length of about 18 nucleotides.

However, decreasing the length of the oligonucleotides in the duplex from 30 to 18 nucleotides decreases the Tm of the duplex and increases the melting point change (ΔTm) obtained by nucleobase mismatches.

The ΔTm obtained for a duplex probe comprising a single nucleobase mismatch is lower than the ΔTm obtained for a probe comprising a single abasic site. Furthermore, the ΔTm obtained for a duplex probe comprising two nucleobase mismatches is lower than the ΔTm obtained for a probe comprising two abasic sites. Accordingly, an increase in the number of abasic sites or mismatches from 1 to 2 incur an increase in ΔTm with about a factor 2-3. Accordingly, in an embodiment, the duplex probe comprises at least 2 abasic sites or mismatches, such as exactly 2 abasic sites or mismatches.

Insertion of an intercalator molecule into the backbone structure of a polynucleotide probe increases the stability of the probe and significantly reduces the melting point decrease obtained, when the intercalator in the probe is positioned complementary to the abasic site in the target sequence. Compared to a probe comprising one mismatch, such insertion of an intercalator significantly decreases ΔTm, by approximately a factor 3-4. Similarly, when comparing the melting point decreases observed from a probe comprising two mismatch sites with the melting point decreases observed from a probe wherein 2 intercalators are inserted into the backbone structure of said polynucleotide probe positioned complementary to the abasic sites in the target sequence, such insertion of intercalators significantly decreases ΔTm by approximately a factor 2, e.g. with a factor 3.

More specifically, an embodiment, one mismatch is present which leads to a ΔTm for a 22-mer duplex of about 4° C. to 13° C., such as with about 7° C. to about 10° C., typically with about 8° C. In another embodiment one abasic site is present which leads to a ΔTm for a 22-mer duplex of about 8° C. to 18° C., such as with about 10° C. to about 16C, typically with about 12-14° C. In another embodiment two mismatches are present which lead to a ΔTm for a 22-mer duplex of about 10° C. to 28° C., such as with about 13° C. to about 25° C., typically with about 18° C. In another embodiment two abasic sites are present which lead to a ΔTm for a 22-mer duplex of at least 15° C., such of at least 17° C., typically at least about 20° C. In another embodiment three mismatches are present which lead to a ΔTm for a 22-mer duplex of at least 20° C., such of at least 23° C., typically at least about 25° C. In another embodiment three abasic sites are present which lead to a ΔTm for a 22-mer duplex of at least 25° C., such of at least 28° C., typically at least about 30° C.

In another specific embodiment, one mismatch is present which leads to a ΔTm for a 30-mer duplex of about 2° C. to 8° C., such as with about 4° C. to about 7° C., typically with about 6° C. In another embodiment one abasic site is present which leads to a ΔTm for a 30-mer duplex of about 8° C. to 12° C., such as with about 9° C. to about 10° C., typically with about 8.5° C. to 9.5° C. In another embodiment two mismatches are present which lead to a ΔTm for a 30-mer duplex of about 10° C. to 17° C., such as with about 11° C. to about 14° C., typically with about 13° C. In another embodiment two abasic sites are present which lead to a ΔTm for a 30-mer duplex of about 15° C. to 24° C., such of about 17° C. to 23° C., typically about 19° C. to about 22° C. In another embodiment three mismatches are present which lead to a ΔTm for a 30-mer duplex of at about 18° C. to 25° C., such of at about 21° C. to 22° C., typically at least about 21.5° C. In another embodiment three abasic sites are present which lead to a ΔTm for a 30-mer duplex of at least 25° C., such of at least 29° C., typically at least about 31° C.

In another embodiment, one intercalator molecule is inserted into the backbone structure of a polynucleotide probe positioned complementary to the abasic site in the target sequence which leads to a ΔTm for a 22-mer duplex of at about 0° C. to 4° C., such of at about 1° C. to 2° C., typically at least about 1.5° C. In another embodiment, two intercalator molecules are inserted into the backbone structure of a polynucleotide probe positioned complementary to the two abasic sites in the target sequence which leads to a ΔTm for a 22-mer duplex of at about 3° C. to 8° C., such of at about 5° C. to 7° C., typically at least about 6° C. In another embodiment, three intercalator molecules are inserted into the backbone structure of a polynucleotide probe positioned complementary to the three abasic sites in the target sequence which leads to a ΔTm for a 22-mer duplex of at about 10° C. to 14° C., such of at about 11° C. to 13° C., typically about 12° C. In another embodiment, one intercalator molecule is inserted into the backbone structure of a polynucleotide probe positioned complementary to the abasic site in the target sequence which leads to a ΔTm for a 30-mer duplex of at about 1° C. to 2° C., typically about 1.5° C. In another embodiment, two intercalator molecules are inserted into the backbone structure of a polynucleotide probe positioned complementary to the two abasic sites in the target sequence which leads to a ΔTm for a 30-mer duplex of at about 3° C. to 7° C., such of at about 4° C. to 5° C., typically at least about 4.5° C. In another embodiment, three intercalator molecules are inserted into the backbone structure of a polynucleotide probe positioned complementary to the three abasic sites in the target sequence which leads to a ΔTm for a 30-mer duplex of at about 6° C. to 11° C., such of at about 8° C. to 9° C., typically about 8.5° C.

The present invention further relates to a method wherein an intercalator molecule has been inserted in more than 10% of the abasic sites in the target polynucleotide such as DNA and/or RNA, such as more than 20%, for example more than 30%, such as more than 40%, for example more than 50%, such as more than 60%, for example more than 70%, such as more than 80%, for example more than 90%, such as more than 95%, for example 100%.

The ratio between the total number of intercalator molecules and the total number of bases in the complementary probe is in one embodiment from 1:50 to 1:2 such as from 1:50 to 1:40, for example 1:40 to 1:30, such as from 1:30 to 1:20, for example 1:20 to 1:10, such as from 1:10 to 1:5, for example 1:5 to 1:2, or any combination of these intervals. One particular embodiment of the present invention relates to a polynucleotide probe wherein the ration between the number of intercalator molecules and the total number of bases in the polynucleotide probe is from 1:50 to 1:2 such as from 1:50 to 1:40, for example 1:40 to 1:30, such as from 1:30 to 1:20, for example 1:20 to 1:10, such as from 1:10 to 1:5, for example 1:5 to 1:2, or any combination of these intervals.

In an embodiment of the method according disclosed herein, the complementary detection probe comprises one or more intercalator molecules. In an embodiment thereof, the complementary detection probe can comprise one or more intercalator molecules such as 1, 2, 3, 4, 5, 6, 7, 8, 9, 10, 11, 12, 13, 14, 15, 16, 17, 18, 19, 20 or more than 20 different or identical intercalator molecules.

The length of the detection probe is in one embodiment selected from the group consisting of 5, 6, 7, 8, 9, 10, 11, 12, 13, 14, 15, 16, 17, 18, 19, 20, 21, 22, 23, 24, 25, 26, 27, 28, 29, 30 and more than 30 bases.

The detection probe can be any polynucleotide probe made of polynucleotide which may be made of a naturally occurring nucleotides or which may be made of nucleotides which are not known to occur naturally or any mixture thereof, such as a DNA, a RNA, a LNA or PNA probe. In one particular embodiment the detection probe is made of polynucleotide which is made of naturally occurring nucleotide, such as RNA or DNA. In one specific embodiment thereof, said detection probe comprises RNA. In another specific embodiment thereof, said detection probe is comprises DNA. In another particular embodiment, the detection probe is made of polynucleotide not known to occur naturally, such as comprising one or more analogue(s) selected from the group consisting of LNA, PNA, PMO, TNA, GNA, oligonucleotide N3′→P5′ phosphoramidates, BNA, α-L-LNA and other constrained nucleotides. In one specific embodiment thereof said detection probe comprises LNA. In one specific embodiment thereof said detection probe comprises PNA. In one specific embodiment thereof said detection probe comprises PMO. In one specific embodiment thereof said detection probe comprises TNA. In one specific embodiment thereof said detection probe comprises GNA. In one specific embodiment thereof said detection probe comprises nucleotide N3′→P5′ phosphoramidates. In one specific embodiment thereof said detection probe comprises BNA. In one specific embodiment thereof said detection probe comprises α-L-LNA. In one specific embodiment thereof said detection probe comprises other constrained nucleotides as defined herein.

The method of the present invention may be used within a wide range of applications such as but not limited to within diagnosing, monitoring antisense therapy, identification of familial relatives, personalized medicine, forensic genetics, quantitative RNA analysis, detection of microorganisms, archaeology and paleopathology, food contamination and environmental pollution. Accordingly, the present invention may be used within for diagnosis purposes and for pharmaceuticals, vetenary medicine, environmental medicine and within quality control of food production.

### Intercalators

An intercalator according to the invention is, as already mentioned, a type of molecule that, like a conventional nucleobase (base), can be inserted in an abasic site of a polynucleotide probe which may be made of a naturally occurring nucleotides or which may be made of nucleotides which are not known to occur naturally or any mixture thereof, such as a DNA probe, a RNA probe, a PNA probe or a LNA probe.

Further to the insertion into an abasic site mentioned above, the intercalator according to the invention may also be capable of fitting itself in between base pairs of double stranded polynucleotide which may be a naturally occurring nucleotides or which may be nucleotides which are not known to occur naturally. In other words, the intercalator of the invention may further be capable of fitting itself into an open space between base pairs of said polynucleotide. Such open space is not provided by an abasic site but is positioned where an open space between base pairs. Such open space is provided e.g. by unwinding of the polynucleotide or in a terminal position of said polynucleotide. The incorporation of an intercalator according to the invention into such open space may be done enzymatically or without use of enzymes. Accordingly, in one embodiment of the method disclosed herein, the one or more intercalator(s) as defined herein is/are inserted into one or more abasic sites, only. In another embodiment of the invention, one or more further intercalators as defined herein is/are positioned in between base pairs of double stranded polynucleotide.

In an embodiment of the invention, the intercalator according to the present invention is not conjugated to a nucleic acid residue.

In another embodiment of the invention, the intercalator disclosed herein does not utilize charge transfer.

In a particular embodiment of the invention, the intercalator is a chemical entity of the general structure X-Y wherein X is an intercalating unit comprising at least one essentially flat conjugated system, which is capable of co-stacking with nucleobases of a nucleic acid; and Y is a linker linking the intercalating unit to the polynucleotide probe which may be made of a naturally occurring nucleotides or which may be made of nucleotides which are not known to occur naturally or any mixture thereof, such as a DNA probe, a RNA probe, a PNA probe or a LNA probe. In a particular embodiment thereof the invention, said essentially flat conjugated system is completely planar.

In a preferred embodiment, the intercalators according to the invention are of the general structure X-Y wherein X is an intercalating unit; and Y is a linker linking the intercalating unit to the polynucleotide probe.

The intercalating unit of the intercalator according to the invention preferably comprises a chemical group selected from the group consisting of polyaromates and heteropolyaromates and even more preferably the intercalating unit essentially consists of a polyaromate or a heteropolyaromate. Most preferably, the intercalator is selected from the group consisting of polyaromates and heteropolyaromates.

Polyaromates or heteropolyaromates according to the present invention may consist of any suitable number of aromatic rings, such as at least 2, such as 2, such as 3, for example 4, such as 5, for example 6, such as 7, for example 8, such as more than 8 aromatic rings.

In one embodiment of the present invention, the size of the intercalating unit is between 20 and 400 Å, such as from 20-40 Å, for example from 40-60 Å, such as from 60-80 Å, for example from 80-100 Å, such as from 100-120 Å, for example from 120-140 Å, such as from 140-160 Å, for example from 160-180 Å, such as from 180-200 Å, for example from 200-220 Å, such as from 220-240 Å, for example from 240-260 Å, such as from 260-280 Å, for example from 280-300 Å, such as from 300-320 Å, for example from 320-340 Å, such as from 340-360 Å, for example from 360-380 Å, such as from 380-400 Å, or any combination of these intervals.

Heteropolyaromates according to the present invention contains at least one aromatic ring wherein at least one carbon atom is replaced by a heteroatom selected from nitrogen and oxygen, such as oxygen, for example nitrogen. Heteropolyaromates according to the invention contains such as at least 2 hetero atoms, such as 2 heteroatoms, such as 3 heteroatoms, for example 4 heteroatoms, such as 5 heteroatoms, for example more than 5 heteroatoms. Heteropolyaromates according to the invention containing more than one heteroatom contains such as one or more oxygen but no other heteroatoms, such as one oxygen and no other heteroatoms, for example one or more nitrogen but no other heteroatoms, for example one nitrogen but no other heteroatoms, such as one or more nitrogen and one or more oxygen but no other heteroatoms.

Polyaromates or heteropolyaromates according to the present invention may be substituted with one or more substituents selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl and/or amido or two adjacent substituents may together form N═C—CH or C═C.

In one preferred embodiment of the present invention the intercalating unit may be selected from the group consisting of polyaromates and heteropolyaromates that are capable of increasing the stability of the polynucleotide duplex structure.

In one preferred embodiment, the intercalating unit is selected from the group consisting of phenanthroline, phenazine, phenanthridine, pyrene, anthracene, naphthalene, phenanthrene, picene, chrysene, naphtacene, benzanthracene, stilbene, porphyrin and any of the aforementioned intercalators substituted with one or more substituents selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl and/or amido or two adjacent substituents may together form N═C—CH or C═C.

In another preferred embodiment, the intercalating unit is selected from the group consisting of bi-cyclic aromatic ringsystems, tricyclic aromatic ringsystems, tetracyclic aromatic ringsystems, pentacyclic aromatic ringsystems and heteroaromatic analogues thereof and substitutions thereof. In a specific embodiment thereof, said intercalating unit is selected from the group consisting of pyrene, phenanthroimidazole and naphthalimide.

In another preferred embodiment, the intercalating unit is selected from the group consisting of modified nucleobases. Non-limiting examples thereof are ^{MPy}U, ^{AMPy}U, ^{Oxo-Py}U and such analogues wherein U is replaced with any of the other nucleobases herein disclosed. One specific embodiment is directed to those intercalating units which are selected from the group consisting of ^{MPy}U, ^{AMPy}U, ^{Oxo-Py}U (Bag et. al, Bioorganic & Medicinal Chemistry Letters 20 (2010) 3227-3230). Particularly preferred is ^{Oxo-Py}U.

Preferably the intercalating unit is of the below formula 1:

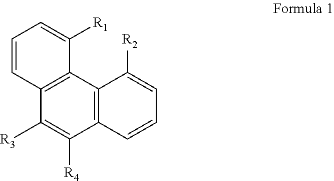

wherein:
R1 is selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl; and
R2 selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl and/or amido; or two adjacent substituents R1 and R2 together form N═C—CH or C═C; and
R3 is selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl; and
R4 selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl and/or amido; or two adjacent substituents R3 and R4 together form N═C—CH or C═C.

Even more preferably, the intercalating unit is of one of the below formulas:

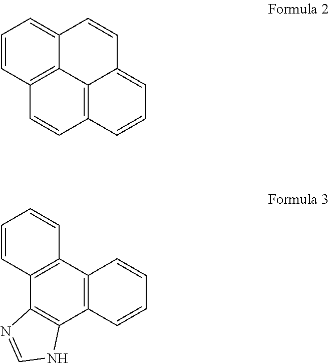

In a specific and preferred embodiment of formula 1, the intercalating unit is of formula 2, i.e. a pyrene moiety.

In a specific and preferred embodiment of formula 1, the intercalating unit is of formula 3.

The intercalating units may be attached to the linker at any available position. However attachment as indicated below is preferred:

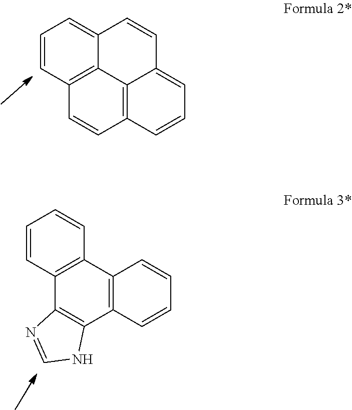

In one preferred embodiment of formula 2, the intercalating unit is attached as indicated in formula 2*.

In one preferred embodiment of formula 3, the intercalating unit is attached as indicated in formula 3*.

The above list of examples is not to be understood as limiting in any way, but only as to provide examples of possible structures for use as intercalating units. In addition, the substitution of one or more chemical groups on each intercalating unit to obtain modified structures is also included in the present invention.

The intercalating unit of the intercalator pseudonucleotide is linked to the backbone unit of the polynucleotide probe by the linker Y. When going from the backbone along the linker to the intercalating unit, then connection between the linker and the intercalating unit is defined as the bond between a linker atom and the first atom being part of the conjugated system of the intercalating unit. Said linker molecule covalently or non-covalently connects the backbone of the probe with the intercalating unit, thereby creating a larger complex consisting of all molecules including the linker molecule.

The linker is the shortest path linking the polynucleotide probe to the intercalating unit. The linker usually consists of a chain of atoms or a branched chain of atoms. Chains can be saturated as well as unsaturated. The linker may also be a ring structure with or without conjugated bonds.

The linker is a bond in an embodiment wherein the intercalating unit is linked directly to the backbone.

In another embodiment, the linker comprises one or more atom(s) or bond(s) between atoms.

In yet another embodiment, the linker may comprise a conjugated system and the intercalating unit may comprise another conjugated system. In this case the linker conjugated system is not capable of costacking with nucleobases in the abasic site.

For example the linker may comprise a chain of m atoms selected from the group consisting of C, O, S, N, P, Se, Si, Ge, Sn and Pb, wherein one end of the chain is connected to the intercalating unit and the other end of the chain is connected to the backbone monomer unit of the polynucleotide probe.

In some embodiments the total length of the linker and the intercalating unit according to the present invention preferably is between 8 and 13 Å. The area normally occupied by two natural bases is 269 Å [Kool, 6]. Accordingly, m should be selected dependent on the size of the intercalating unit. I.e. m should be relevatively large, when the intercalator is small and m should be relatively small when the intercalator is large.

For most purposes m will be an integer from 1 to 13, such as from 1-12, such as from 1-11, such as from 1-10, such as from 1-9, such as from 1-8, such as from 1-7, such as from 1-6, such as from 1-5, such as from 1-4. As described above the linker may be an alkyl such as an unsaturated chain or another system involving conjugated bonds. For example the linker may comprise cyclic conjugated structures. Preferably, m is from 1 to 4 when the linker is a saturated chain and from 7-13, such as from 9-11 when the linker comprises a cyclic conjugated structure.

In one embodiment of the invention, the size of the linker is between 20 and 400 Å, such as from 20-40 Å, for example from 40-60 Å, such as from 60-80 Å, for example from 80-100 Å, such as from 100-120 Å, for example from 120-140 Å, such as from 140-160 Å, for example from 160-180 Å, such as from 180-200 Å, for example from 200-220 Å, such as from 220-240 Å, for example from 240-260 Å, such as from 260-280 Å, for example from 280-300 Å, such as from 300-320 Å, for example from 320-340 Å, such as from 340-360 Å, for example from 360-380 Å, such as from 380-400 Å, or any combination of these intervals.

In another embodiment the linker consists of from 1-6 C atoms, from 0-3 of each of the following atoms O, S, N. More preferably the linker consists of from 1-6 C atoms and from 0-1 of each of the atoms O, S, N.

The chain of the linker may be substituted with one or more atoms selected from the group consisting of halogen, iodo, mercapto, thio, cyano, alkylthio, heterocycle, aryl, heteroaryl, carboxyl, carboalkoyl, alkyl, alkenyl, alkynyl, nitro, amino, alkoxyl and amido.

More particularly, the linker may consist of a chain comprising atoms selected from the group consisting of C, O, S, N, P, Se, Si, Ge, Sn and Pb. Such chain may comprise one or more alkyl groups, such as C₁₋₆-alk(en/yn)yl, such as C₁₋₆-alkyl, for example an unbranched alkyl chain, wherein one end of the chain is connected to the intercalating unit and the other end of the chain is connected to the backbone monomer unit of the polynucleotide probe and wherein each C is substituted with 2 H. In a preferred embodiment, said chain contains one or more oxygen atoms (O) further to the alkyl chains mentioned. In an embodiment, said chain is C₁₋₆-alk(en/yn)yl-O—C₁₋₆-alk(en/yn)yl, such as C₁₋₆-alkyl-O—C₁₋₆-alkyl. Preferably, said unbranched alkyl chain is from 1 to 5 atoms long, such as from 1 to 4 atoms long, such as from 1 to 3 atoms long, such as from 2 to 3 atoms long. In a preferred embodiment the linker is CH₂—O—CH₂.

More particularly, the linker may comprise a ring structure and a chain structure comprising atoms selected from the group consisting of C, O, S, N, P, Se, Si, Ge, Sn and Pb. Non limiting examples of the ring structured moiety comprised therein are aryl or C₃₋₈-cycloalk(en)yl. In a particular embodiment the linker is an aryl, such as a phenyl group. The chain structured moiety comprised therein may comprise one or more alkyl groups, such as C₁₋₆-alk(en/yn)yl, such as C₁₋₆-alkyl, for example an unbranched alkyl chain, wherein one end of the chain is connected to the intercalating unit and the other end of the chain is connected to the backbone monomer unit of the polynucleotide probe and wherein each C is substituted with 2 H. In a preferred embodiment, said chain contains one or more oxygen atoms (O) further to the alkyl chains mentioned. In an embodiment, such linker is selected from the group consisting of aryl-O—C₁₋₆-alk(en/yn)yl and C₁₋₆-alk(en/yn)yl-aryl-C₁₋₆-alk(en/yn)yl-O—C₁₋₆-alk(en/yn)yl. In particular embodiments of the invention, said linker is a aryl-O—C₁₋₆-alk(en/yn)yl, such as aryl-O—C₁₋₆-alk(en/yn)yl, such as aryl-O—C₁₋₆-alkyl. For example such linker may be substituted with one or more substituents selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl and/or amido. In a preferred embodiment, such linker is phenyl-O-ethyl. In another preferred embodiment, such linker is naphtyl-O-ethyl. In particular embodiments of the invention, said linker is a C₁₋₆-alk(en/yn)yl-aryl-C₁₋₆-alk(en/yn)yl-O—C₁₋₆-alk(en/yn)yl, such as C₁₋₆-alk(en/yn)yl-aryl-C₁₋₆-alk(en/yn)yl-O—C₁₋₆-alk(en/yn)yl, such as C₁₋₆-alkyl-aryl-C₁₋₆-alkyl-O—C₁₋₆-alkyl. In a preferred embodiment, such as linker is ethynyl-phenyl-methyl-O-methyl.

More particularly, the linker may be a ring structure comprising atoms selected from the group consisting of C, O, S, N, P, Se, Si, Ge, Sn and Pb. Non limiting examples of such ring structures are aryl C₃₋₆-cycloalk(en)yl. For example such linker may be substituted with one or more substituents selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl and/or amido. In a particular embodiment the linker is an aryl, such as aryl, such as phenyl.

In one embodiment, the linker according to the invention is hydrophobic in nature. In another embodiment of the invention the linker is hydrophilic in nature.

In one embodiment, the linker according to the invention is flexible in nature. In another embodiment, the linker is rigid in nature.

The area occupied by pyrene is 220 Å [6]. In one embodiment of the invention, the intercalating unit is pyrene and the size of the linker is 20 and 200 Å, such as from 20-40 Å, for example from 40-60 Å, such as from 60-80 Å, for example from 80-100 Å, such as from 100-120 Å, for example from 120-140 Å, such as from 140-160 Å, for example from 160-180 Å, such as from 180-200 Å, or any combination of these intervals.

In an embodiment of the invention wherein the intercalator is pyrene, m is preferably an integer from 1 to 11, such as from 1-10, such as from 1-9, such as from 1-8, such as from 1-7, such as from 1-6, such as from 1-5, such as from 1-4, such as from 1-3.

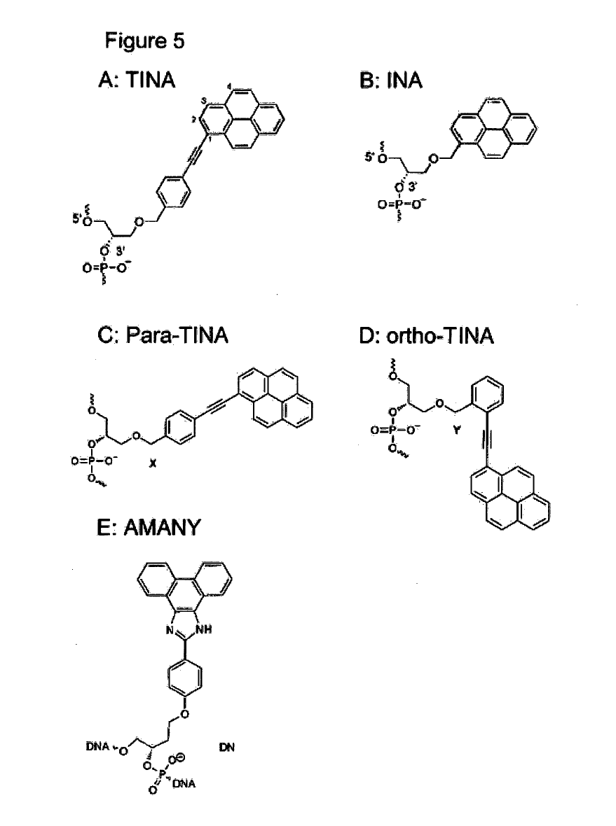

In an embodiment, the intercalating unit is pyrene and the linker is C₁₋₆-alk(en/yn)yl-O—C₁₋₆-alk(en/yn)yl, such as C₁₋₆-alkyl-O—C₁₋₆-alkyl, such as CH₂—O—CH₂. In a preferred embodiment the intercalating unit together with the linker form the complex designated INA in FIG. 5.

In another embodiment, the intercalating unit is pyrene and the linker is C₁₋₆-alk(en/yn)yl-aryl-C₁₋₆-alk(en/yn)yl-O—C₁₋₆-alk(en/yn)yl, such as C₁₋₆-alk(en/yn)yl-aryl-C₁₋₆-alk(en/yn)yl-O—C₁₋₆-alk(en/yn)yl, such as C₁₋₆-alkyl-aryl-C₁₋₆-alkyl-O—C₁₋₆-alkyl, such as ethynyl-phenyl-methyl-O-methyl. In a preferred embodiment the intercalating unit together with the linker form the complex designated TINA in FIG. 5. In another preferred embodiment the intercalating unit together with the linker form the complex designated para-TINA in FIG. 5. In a preferred embodiment the intercalating unit together with the linker form the complex designated ortho-TINA in FIG. 5.

In yet another embodiment, the intercalating unit is of formula 3 and the linker is aryl-O—C₁₋₆-alk(en/yn)yl, such as aryl-O—C₁₋₆-alk(en/yn)yl, such as aryl-O—C₁₋₆-alkyl, such as phenyl-O-ethyl. In another preferred embodiment, the intercalating unit together with the linker form the complex designated Amany in FIG. 5.

In a preferred embodiment, the insertion into an abasic site of a complementary DNA or RNA structure of a intercalator according to the invention results in increased stability of the polynucleotide duplex structure.

In one embodiment the size of the intercalator molecule is between 20 and 400 Å, such as from 20-40 Å, for example from 40-60 Å, such as from 60-80 Å, for example from 80-100 Å, such as from 100-120 Å, for example from 120-140 Å, such as from 140-160 Å, for example from 160-180 Å, such as from 180-200 Å, for example from 200-220 Å, such as from 220-240 Å, for example from 240-260 Å, such as from 260-280 Å, for example from 280-300 Å, such as from 300-320 Å, for example from 320-340 Å, such as from 340-360 Å, for example from 360-380 Å, such as from 380-400 Å, or any combination of these intervals.

In one embodiment more than one type of intercalator molecule is used in each probe such as 2, 3, 4, 5 or more than 5 different types. Accordingly, in an embodiment, the polynucleotide probe, such as the complementary probe, comprises more than one type of intercalator molecules such as 2, 3, 4, 5 or more than 5 different types of intercalator molecules.

In a preferred embodiment the intercalating unit together with the linker form a complex selected from the group consisting of TINA, INA, ortho-TINA, para-TINA, and AMANY as illustrated in FIG. 5.

A particular embodiment of the present invention relates to a method comprising use of one or more intercalator molecules which can be selected from the group consisting of TINA, INA, ortho-TINA, para-TINA, and AMANY [3, 4, 5, 6].

One further embodiment of the present invention relates to a polynucleotide probe wherein the one or more intercalator molecules is selected from the group consisting of TINA, INA, ortho-TINA, para-TINA, and AMANY. Yet one further embodiment of the present invention relates to a polynucleotide probe wherein the two or more intercalator molecules are selected from the group consisting of TINA, INA, ortho-TINA, para-TINA, and AMANY. A specific embodiment thereof, relates to a polynucleotide probe wherein the intercalator molecule is TINA. A further specific embodiment, relates to a polynucleotide probe wherein the intercalator molecule is INA. A further specific embodiment, relates to a polynucleotide probe wherein the intercalator molecule is ortho-TINA. A further specific embodiment, relates to a polynucleotide probe wherein the intercalator molecule is para-TINA. A further specific embodiment, relates to a polynucleotide probe wherein the intercalator molecule is AMANY.

TINA, INA and AMANY are intercalators designed to stabilize Hoogsteen triplex DNA, but surprisingly, they can also be used to stabilize double stranded DNA.

The intercalator molecules increase the melting temperature (Tm) and ΔTm of antiparallel duplex formations in hybridizations assays.

### Supports

The complementary probe may, as previously mentioned, be connected to a support. Accordingly, in one embodiment, the polynucleotide probe, i.e. the complementary probe, is connected to a support, such as a solid support. The support can be a solid, semi-solid or soluble support. The support can be any suitable support disclosed in the prior art. In an embodiment of the present invention, the polynucleotide probe, such as the complementary probe, is connected to a support. In an embodiment thereof, said support is a solid support. In a further embodiment thereof, the support is selected from the group consisting of particulate matters, beads, magnetic beads, non-magnetic beads, polystyrene beads, magnetic polystyrene beads, sepharose beads, sephacryl beads, polystyrene beads, agarose beads, polysaccharide beads, and polycarbamate beads.

Non-limiting examples of supports are listed herein below:

Poly(ether ether ketone) (PEEK), PP (polypropylene), PE (polyethylene), Poly(ethylene terephthalate) (PET), Poly(vinyl chloride) (PVC), Polyamide/nylon (PA), Polycarbonate (PC), Cyclic olefin copolymer (COC), Filter paper, Cotton, Cellulose, Poly(4-vinylbenzyl chloride) (PVBC), Poly(vinylidene fluoride) (PVDF), Polystyrene (PS), Toyopearl®, Hydrogels, Polyimide (PI), 1,2-Polybutadiene (PB), LSR (Liquid silicon rubber), poly(dimethylsiloxane) (PDMS), fluoropolymers-and copolymers (e.g. poly(tetrafluoroethylene) (PTFE), Perfluoroethylene propylene copolymer (FEP), Ethylene tetrafluoroethylene (ETFE)), poly(methyl methacrylate) (PMMA), Nanoporous materials, Membranes, Mesostructured cellular foam (MCF), and singlewall or multiwall Carbon Nanotubes (SWCNT, MWCNT), particulate matters, beads, magnetic beads, non-magnetic beads, polystyrene beads, magnetic polystyrene beads, sepharose beads, sephacryl beads, polystyrene beads, agarose beads, polysaccharide beads, and polycarbamate beads.

In one specific embodiment, the support is selected from the group of Polymeric or organic substrates such as from the group consisting of Poly(ether ether ketone) (PEEK), PP (polypropylene), PE (polyethylene), Poly(ethylene terephthalate) (PET), Poly(vinyl chloride) (PVC), Polyamide/nylon (PA), Polycarbonate (PC), Cyclic olefin copolymer (COC), Filter paper, Cotton, Cellulose, Poly(4-vinylbenzyl chloride) (PVBC), Poly(vinylidene fluoride) (PVDF), Polystyrene (PS), Toyopearl®, Hydrogels, Polyimide (PI), 1,2-Polybutadiene (PB), LSR (Liquid silicon rubber), poly(dimethylsiloxane) (PDMS), fluoropolymers-and copolymers (e.g. poly(tetrafluoroethylene) (PTFE), Perfluoroethylene propylene copolymer (FEP), Ethylene tetrafluoroethylene (ETFE)), poly(methyl methacrylate) (PMMA).

In another specific embodiment, the support is selected from the group consisting of Nanoporous materials, Membranes, Mesostructured cellular foam (MCF), and singlewall or multiwall Carbon Nanotubes (SWCNT, MWCNT).

In yet another specific embodiment, the support is selected from the group consisting of particulate matters, beads, magnetic beads, non-magnetic beads, polystyrene beads, magnetic polystyrene beads, sepharose beads, sephacryl beads, polystyrene beads, agarose beads, polysaccharide beads, and polycarbamate beads.

In further embodiments, the solid support is selected from the group consisting of microtiter plate, other plate formats, reagent tubes, glass slides and other supports for use in array or microarray analysis, tubings or channels of micro fluidic chambers or devices and Biacore chips.

### Labels

As previously mentioned the detection probe and/or the complementary DNA probe can comprise one or more labels such 1, 2, 3, 4, 5, 6, 7, 8, 9, 10, 11, 12, 13, 14, 15, 16, 17, 18, 19, 20 or more than 20 identical or different labels.

Accordingly, in one embodiment, the invention relates to a polynucleotide probe comprising one or more labels. A further embodiment provides the method disclosed herein comprising use of a detection probe comprising one or more labels. In one further embodiment, the method disclosed herein comprises use of a complementary probe comprising one or more labels.

The one or more labels can be any state-of-the art label such as one or more labels selected from the consisting of biotin, a fluorescent label, 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, dyes, Cy2, Cy3, and Cy5, PerCP, phycobiliproteins, R-phycoerythrin (RPE), allophycoerythrin (APC), Texas Red, Princeston Red, Green fluorescent protein (GFP) and analogues thereof, conjugates of R-phycoerythrin or allophycoerythrin, inorganic fluorescent labels based on semiconductor nanocrystals (like quantum dot and Qdot™ nanocrystals), time-resolved fluorescent labels based on lanthanides like Eu3+ and Sm3+, haptens, DNP, digoxiginin, enzymic labels, horse radish peroxidase (HRP), alkaline phosphatase (AP), beta-galactosidase (GAL), glucose-6-phosphate dehydrogenase, beta-N-acetyl-glucosaminidase, β-glucuronidase, invertase, Xanthine Oxidase, firefly luciferase and glucose oxidase (GO), luminiscence labels, luminol, isoluminol, acridinium esters, 1,2-dioxetanes, pyridopyridazines, radioactivity labels, isotopes of iodide, isotopes of cobalt, isotopes of elenium, isotopes of tritium, and isotopes of phosphor.

In one preferred embodiment a biotin label is used. Biotin can be detected by use of streptavidin-R-phycoerythrine.

In an embodiment, the method of the present invention comprises one or more washing steps prior to and/or after addition of the detection probe.

The label can be detected by any suitable method disclosed in the prior art.

### Detection of Multiple Target DNA Sequences

One specific embodiment of the present invention relates to detection of multiple target polynucleotide such as target DNA, LNA-modified DNA, RNA, LNA-modified RNA and/or PNA, e.g. DNA and/or RNA sequences by use of the method disclosed herein above. Multiple target polynucleotide such as DNA, LNA-modified DNA, RNA, LNA-modified RNA and/or PNA, e.g. target DNA and/or RNA sequences can in one embodiment be tested by use of multiple complementary probes in an array format or microtiter plate format.

In one further specific embodiment the total number of target polynucleotide, such as different target polynucleotide, such as DNA, LNA-modified DNA, RNA, LNA-modified RNA and/or PNA, e.g. target DNA and/or RNA sequences that are captured can be selected from the group consisting of from 1, 2-5, 5-10, 10-15, 15-20, 20-25, 25-30, 30-35, 35-40, 40-45, 45-50, 50-55, 55-60, 60-65, 65-70, 70-75, 75-80, 80-85, 85-90, 95-100, 100-150, 150-200, 200-300, 300-500, 500-1000 and more than 1000, or any combination of these intervals.

### Uses of the Method of the Invention

The present invention is useful within a wide range of screening applications involving the identification and capture of genetic material, such as e.g. within one or more uses selected from the group consisting of:
diagnosing such as of cancer or hereditary diseases other than cancer or in the field of prenatal diagnosis;
monitoring antisense therapy;
identification of familial relatives;
personalized medicine;
forensic genetics;
quantitative RNA analysis;
detection of microorganisms;
archaeology and paleopathology;
food contamination; and
environmental pollution.

### Diagnosing

The present invention can be used in diagnosis of one or more diseases. The diseases can be diagnosed by detection of target polynucleotide such as DNA and/or RNA from the genome of an individual that is tested or by detection of target polynucleotide which may be made of a naturally occurring nucleotides or which may be made of nucleotides which are not known to occur naturally or any mixture thereof, such as DNA and/or RNA, which is not derived from the genome of the individual that is tested. Accordingly, in an embodiment, the present invention relates to a method for diagnosing one or more diseases comprising use of the method disclosed herein. In one further embodiment, the diagnosis comprises detection of target polynucleotide, such as target DNA and/or RNA, from the genome of an individual that is tested. In another embodiment, the diagnosis comprises detection of target polynucleotide, such as target DNA and/or RNA, which is not derived from the genome of an individual that is tested.

The present invention can be used to diagnose any disease where a specific target polynucleotide which may be made of a naturally occurring nucleotides or which may be made of nucleotides which are not known to occur naturally or any mixture thereof, such as DNA and/or RNA sequence, is known.

In an embodiment, the present invention is used to diagnose one or more diseases such as hereditary diseases, cancer and infectious disease, headaches and other diseases wherein the method of the invention may be useful. Further uses of the present invention is within the field of personalized medicine e.g., but not limited to in cancer treatment.

An embodiment of the invention relates to the use of the method as disclosed herein for the diagnosis of an individual that may have signs or symptoms of the disease in question or who may be without signs or symptoms of that disease. In a particular embodiment of the invention, said individual has signs or symptoms of the disease tested for. When a person has signs or symptoms of the disease tested for, said person may e.g. suffer from an infection or from a hereditary disorder or disease. In another particular embodiment of the invention, said individual has no signs or symptoms of the disease tested for. When testing is performed on persons without any clinical sign of disease, the intention is e.g. to identify a disease screened for at an early stage, thus enabling earlier intervention and management in the hope to reduce mortality and suffering from a disease. Several types of such testing exist: ‘universal screening’ involves screening of all individuals in a certain category (for example, all children of a certain age) and ‘case finding’ involves screening a smaller group of people based on the presence of risk factors such as for example, because a family member has been diagnosed with a hereditary disease. One specific embodiment of the invention relates to the use of the as method disclosed herein within universal screening. Another specific embodiment of the invention relates to the use of the method disclosed herein within case finding.

Human and animal samples include faeces, blood, semen, cerebrospinal fluid (CSF), sputum, vaginal fluid, urine, saliva, hair, other bodily fluids, tissue samples, whole organs, sweat, tears, skin cells, hair, bone, teeth or appropriate fluid or tissue from personal items (e.g. toothbrush, razor, etc.) or from samples (e.g. sperm or biopsy tissue or liquid) or other sub-structures of humans or animals. The sample therefore may be a solid, semi-solid or a fluent sample.

### Diagnosis of Cancer

In one preferred embodiment the present invention is used to diagnose one or more diseases such as cancer. The cancer can be diagnosed by detection of target polynucleotide such as DNA and/or RNA from the genome of an individual that is tested.

The cancer to be diagnosed may be in its early phase or it may be at a large stage of the disease. In an embodiment of the invention the cancer to be diagnosed is preferably in one of its earlier stages such as in its benign or premalignant stage. In another embodiment of the invention the cancer to be diagnosed is preferably on stadium 1 or 2. In an embodiment thereof, the cancer to be diagnosed is benign. In another embodiment thereof the cancer to be diagnosed is malignant. In a preferred embodiment thereof, the cancer to be diagnosed is in its premalignant stage. In yet a further embodiment thereof, the cancer to be diagnosed is on stadium 4. In yet a further embodiment thereof, the cancer to be diagnosed is on stadium 3. In yet a further embodiment thereof, the cancer to be diagnosed is on stadium 2. In a preferred embodiment thereof, the cancer to be diagnosed is on stadium 1.

In an embodiment, different types of cancer are diagnosed by the method according to the present invention, such as e.g. those which are selected from the group listed in Table A herein below:

TABLE A
Types of cancer

Bone cancer - including Ewing's Sarcoma, Osteosarcoma, Chondrosarcoma
Brain and CNS tumors - including Acoustic Neuroma, Spinal Cord Tumours
Breast cancer- including male breast cancer and Ductal Carcinoma in situ
Colorectal cancer - and anal cancer
Endocrine cancers - including Adrenocortical Carcinoma, Pancreatic Cancer, Pituitary
Cancer, Thyroid Cancer, Parathyroid Cancer, Thymus Cancer, Multiple Endocrine
Neoplasia, Other Endocrine cancers.
Gastrointestinal cancers - including Stomach (Gastric) Cancer, Esophageal Cancer,
Small Intestine Cancer, Gall Bladder Cancer, Liver Cancer, Extra-Hepatic Bile Duct
Cancer, Gastrointestinal Carcinoid Tumour
Genitourinary cancers - including Testicular Cancer, Penile Cancer, Prostate Cancer
Gynaecological cancers - including Cervical Cancer, Ovarian Cancer, Vaginal Cancer,
Uterus/Endometrium Cancer, Vulva Cancer, Gestational Trophoblastic Cancer,
Fallopian Tube cancer, Uterine sarcoma
Head and Neck Cancer - including Oral cavity, Lip, Salivary gland Cancer, Larynx,
hypopharynx, oropharynx Cancer, Nasal, Paranasal, Nasopharynx Cancer
Leukaemia - including Childhood Leukaemia, Acute Lymphocytic Leukaemia, Acute
Myeloid Leukaemia, Chronic Lymphocytic Leukaemia, Chronic Myeloid Leukaemia,
Hairy Cell Leukaemia, BMT for Leukaemia, Patient's Home Pages, Acute
Promyelocytic Leukemia, Plasma Cell Leukaemia
Haematological disorders - including Multiple myeoloma, Myelodysplastic Syndromes,
Myeloproliferative Disorders, Aplastic Anaemia, Fanconi Anaemia, Waldenstrom's
Macroglobulinemia
Lung cancer - including Small Cell Lung Cancer, Non-Small Cell Lung Cancer
Lymphoma - including Hodgkin's Disease, Non-Hodgkin's Lymphoma, Aids Related
Lymphoma
Eye cancer - including Retinoblastoma, IntraOcular Melanoma
Skin cancer - including Melanoma, squamous cell carcinoma, basal cell carcinoma
Soft tissue - including Soft tissue sarcoma, Kaposi's sarcoma
Urinary system cancer - including Kidney Cancer, Wilm's Tumour, Bladder Cancer,
Urethral Cancer, Transitional Cell Cancer

In one preferred embodiment the present invention relates to diagnosis of colon cancer. This diagnosis can be performed by detection of target polynucleotide such as DNA in a faeces sample from the individual to be tested.

In another preferred embodiment the present invent is used to diagnose a neoplastic disease, such as cancer, characterized by one or more mutations in one or more genes or genes encoding proteins, such as in one or more of those listed in Table B herein below and in [2].

TABLE B
Genes/proteins involved in neoplastic diseases

101F6, ABR, ADPRTL3, ANP32C, ANP32D, APC2, APC, ARF, ARHGAP8, ARHI,
AT1G14320, ATM, ATP8A2, AXUD1, BAP1, BECN1, BIN1, BRCA1, BRCA2, BTG1,
BTG2, C1orf11, C5orf4, C5orf7, Cables, CACNA2D2, CAP-1, CARS, CAV1, CD81,
CDC23, CDK2AP1, CDKN1A, CDKN1C, CDKN2A, CDKN2B, CDKN2X, Ciao1-
pending, CLCA2, CREBL2, CTNNA1, CUL2, CW17R, DAB2, DAF-18, D-APC, DBC2,
DCC, DDX26, DEC1, DLC1, DLEC1, DLEU1, DLEU2, DLG1, DLGH1, DLGH3,
DMBT1, DNAJA3, DOC-1, DPC4, DPH2L, EGR1, FABP3, FAT, FGL1, FHIT,
FLJ10506, FOXD1, FOXP1, FT, FUS1, FUS2, GAK, GAS1, GAS11, GLD-1, GLTSCR1,
GLTSCR2, GRC5, GRLF1, HDAC3, HEMK, HIC1, HRG22, HSAL2, HTS1, HYAL1,
HYAL2, IFGBP7, IGSF4, ING1, ING1L, ING4, I(2)tid, I(3)mbn, I(3)mbt, LAPSER1,
LATS1, LATS2, LDOC1, LOH11CR2A, LRP1B, LUCA3, MAD, MAP2K4, MAPKAPK3,
MCC, MDC, MEN1, ML-1, MLH1, MRVI1, MTAP, MXI1, NAP1L4, NBR2, NF1, NF2,
NORE1, NPR2L, NtRb1, OVCA2, PDGFRL, PHEMX, pHyde, PIG8, PIK3CG, PINX1,
PLAGL1, PRDM2, PTCH, PTEN, PTPNI3, PTPRG, RASSF1, RB1, RBBP7, RBX1,
RBM6, RECK, RFP2, RIS1, RPL10, RPS29, RRM1, S100A2, SEMA3B, SF1, SFRP1,
SLC22A1L, SLC26A3, SMARCA4, ST7, ST7L, ST13, ST14, STIM1, TCEB2, THW,
TIMP3, TP53, TP63, TRIM8, TSC2, TSG101, TSSC1, TSSC3, TSSC4, VHL, Vhlh,
WFDC1, WIT-1, WT1, WWOX.

In another embodiment the present invention can be used for diagnosis of cancer wherein the cancer is characterized by one or more tumor antigens selected from the group listed in Table C herein below. Accordingly one embodiment relates to the detection of target polynucleotide such as DNA derived from one or more tumor antigens such as the ones listed in Table C herein below.

TABLE C
Tumor antigens

Alpha-actinin-4, ARTC1, BCR-ABL fusion protein (b3a2), B-RAF, CASP-5, CASP-8,
beta-catenin, Cdc27, CDK4, CDKN2A, COA-1, dek-can fusion protein, EFTUD2,
Elongation factor 2, ETV6-AML1 fusion protein, FLT3-ITD, FN1, GPNMB, LDLR-
fucosyl transferase AS fusion protein, HLA-A2^{d}, HLA-A11^{d}, hsp70-2, KIAAO205,
MART2, ME1, MUM-1^{f}, MUM-2, MUM-3, neo-PAP, Myosin class I, NFYC, OGT, OS-9,
P53, pml-RARalpha fusion protein, PRDX5, PTPRK, K-ras, N-ras, RBAF600, SIRT2,
SNRPD1, SYT-SSX1 or -SSX2 fusion protein, Triosephosphate Isomerase, BAGE-1,
GAGE-1,2,8, GAGE-3,4,5,6,7, GnTV^{f}, HERV-K-MEL, KK-LC-1, KM-HN-1, LAGE-1,
MAGE-A1, MAGE-A2, MAGE-A3, MAGE-A4, MAGE-A6, MAGE-A9, MAGE-A10,
MAGE-A12, MAGE-C2, mucin^{k}, NA-88, NY-ESO-1/LAGE-2, SAGE, Sp17, SSX-2,
SSX-4, TRAG-3, and TRP2-INT2^{g}.

In another embodiment the present invention can be used for diagnosis of cancer by detection of target polynucleotide such as DNA derived from one or more tumor antigens or oncogenes such as the ones listed in Table D herein below.

TABLE D

| Symbol | Description |
|---|---|
| ABL1 | v-abl Abelson murine leukemia viral oncogene homolog 1 |
| ABL2 | v-abl Abelson murine leukemia viral oncogene homolog 2 |
| | (arg, Abelson-related gene) |
| AKT1 | v-akt murine thymoma viral oncogene homolog 1 |
| AKT2 | v-akt murine thymoma viral oncogene homolog 2 |
| APC | adenomatosis polyposis coli |
| ARAF1 | v-raf murine sarcoma 3611 viral oncogene homolog 1 |
| ARHA | ras homolog gene family, member A |
| ARHB | ras homolog gene family, member B |
| ARHC | ras homolog gene family, member C |
| AXL | AXL receptor tyrosine kinase |
| BCL2 | B-cell CLL/lymphoma 2 |
| BCL3 | B-cell CLL/lymphoma 3 |
| BCR | breakpoint cluster region |
| BLYM | avian lymphoma virus-derived transforming sequence |
| BMI1 | murine leukemia viral (bmi-1) oncogene homolog |
| BRAF | v-raf murine sarcoma viral oncogene homolog B1 |
| BRCA1 | breast cancer 1, early onset |
| BRCA2 | breast cancer 2, early onset |
| CBL | Cas-Br-M (murine) ecotropic retroviral transforming sequence |
| CCND1 | cyclin D1 (PRAD1: parathyroid adenomatosis 1) |
| CDH1 | cadherin 1, E-cadherin (epithelial) |
| CDK4 | cyclin-dependent kinase 4 |
| CDKN1A | cyclin-dependent kinase inhibitor 1A (p21, Cip1) |
| CDKN1C | cyclin-dependent kinase inhibitor 1C (p57, Kip2) |
| CDKN2A | cyclin-dependent kinase inhibitor 2A (melanoma, p16, inhibits CDK4) |
| CDKN2B | cyclin-dependent kinase inhibitor 2B (p15, inhibits CDK4) |
| CHES1 | checkpoint suppressor 1 |
| COT | cot (cancer Osaka thyroid) oncogene |
| CRK | v-crk avian sarcoma virus CT10 oncogene homolog |
| CRKL | v-crk avian sarcoma virus CT10 oncogene homolog-like |
| CSF1R | colony stimulating factor 1 receptor, formerly McDonough feline |
| | sarcoma viral (v-fms) oncogene homolog |
| D10S170 | DNA segment, single copy, probe pH4 (transforming sequence, thyroid-1, |
| DCC | deleted in colorectal carcinoma |
| DDX6 | DEAD/H (Asp-Glu-Ala-Asp/His) box polypeptide 6 (RNA helicase, 54 kD) |
| E2F1 | E2F transcription factor 1 |
| EGFR | epidermal growth factor receptor (avian erythroblastic leukemia viral |
| | (v-erb-b) oncogene homolog) |
| EIF3S6 | eukaryotic translation initiation factor 3, subunit 6 (48 kD) |
| ELE1 | RET-activating gene ELE1 |
| ELK1 | ELK1, member of ETS oncogene family |
| ELK3 | ELK3, ETS-domain protein (SRF accessory protein 2) NOTE: |
| | Symbol and name provisional. |
| EMP1 | epithelial membrane protein 1 |
| EMS1 | ems1 sequence (mammary tumor and squamous cell carcinoma- |
| | associated (p80/85 src substrate) |
| EPHA1 | EphA1 |
| EPHA3 | EphA3 |
| ERBAL2 | v-erb-a avian erythroblastic leukemia viral oncogene homolog-like 2 |
| ERBB2 | v-erb-b2 avian erythroblastic leukemia viral oncogene homolog 2 |
| | (neuro/glioblastoma derived oncogene homolog) |
| ERBB3 | v-erb-b2 avian erythroblastic leukemia viral oncogene homolog 3 |
| ERBB4 | v-erb-a avian erythroblastic leukemia viral oncogene homolog-like 4 |
| ERG | v-ets avian erythroblastosis virus E26 oncogene related |
| ETS1 | v-ets avian erythroblastosis virus E26 oncogene homolog 1 |
| ETS2 | v-ets avian erythroblastosis virus E26 oncogene homolog 2 |
| ETV3 | ets variant gene 3 |
| ETV6 | ets variant gene 6 (TEL oncogene) |
| EVI1 | ecotropic viral integration site 1 |
| EWSR1 | Ewing sarcoma breakpoint region 1 |
| FAT | FAT tumor suppressor (Drosophila) homolog |
| FER | fer (fps/fes related) tyrosine kinase (phosphoprotein NCP94) |
| FES | feline sarcoma (Snyder-Theilen) viral (v-fes)/Fujinami avian sarcoma |
| | (PRCII) viral (v-fps) oncogene homolog |
| FGF3 | fibroblast growth factor 3 (murine mammary tumor virus integration |
| | site (v-int-2) oncogene homolog) |
| FGF4 | fibroblast growth factor 4 (heparin secretory transforming protein 1, |
| | Kaposi sarcoma oncogene) |
| FGF6 | fibroblast growth factor 6 |
| FGR | Gardner-Rasheed feline sarcoma viral (v-fgr) oncogene homolog |
| FKHL1 | forkhead (Drosophila)-like 1 |
| FLI1 | Friend leukemia virus integration 1 |
| FLT1 | fms-related tyrosine kinase 1 (vascular endothelial growth |
| | factor/vascular permeability factor receptor) |
| FOS | v-fos FBJ murine osteosarcoma viral oncogene homolog |
| FOSL1 | FOS-like antigen-1 |
| FOSL2 | FOS-like antigen 2 |
| FYN | FYN oncogene related to SRC, FGR, YES |
| GLI | glioma-associated oncogene homolog (zinc finger protein) |
| GLI2 | GLI-Kruppel family member GLI2 |
| GLI3 | GLI-Kruppel family member GLI3 (Greig cephalopolysyndactyly |
| | syndrome) |
| GRO1 | GRO1 oncogene (melanoma growth stimulating activity, alpha) |
| GRO2 | GRO2 oncogene |
| GRO3 | GRO3 oncogene |
| HCK | Hemopoietic cell kinase |
| HKR3 | GLI-Kruppel family member HKR3 |
| HRAS | v-Ha-ras Harvey rat sarcoma viral oncogene homolog |
| IRF4 | interferon regulatory factor 4 |
| JUN | v-jun avian sarcoma virus 17 oncogene homolog |
| JUNB | jun B proto-oncogene |
| JUND | jun D proto-oncogene |
| KAI1 | kangai 1 (suppression of tumorigenicity 6, prostate; CD82 antigen |
| | (R2 leukocyte antigen, antigen detected by monoclonal and antibody IA4)) |
| KIT | v-kit Hardy-Zuckerman 4 feline sarcoma viral oncogene homolog |
| KRAS2 | v-Ki-ras2 Kirsten rat sarcoma 2 viral oncogene homolog |
| LCK | lymphocyte-specific protein tyrosine kinase |
| LTA | Lymphotoxin alpha (TNF superfamily, member 1) |
| LTB | Lymphotoxin beta (TNF superfamily, member 3) |
| LYN | v-yes-1 Yamaguchi sarcoma viral related oncogene homolog |
| M1S1 | membrane component, chromosome 1, surface marker 1 (40 kD |
| | glycoprotein, identified by monoclonal antibody GA733) |
| M4S1 | membrane component, chromosomal 4, surface marker (35 kD |
| | glycoprotein) |
| MADH4 | MAD (mothers against decapentaplegic, Drosophila) homolog 4 |
| MAF | v-maf musculoaponeurotic fibrosarcoma (avian) oncogene homolog |
| MAFG | v-maf musculoaponeurotic fibrosarcoma (avian) oncogene family, |
| | protein G |
| MAS1 | MAS1 oncogene |
| MAX | MAX protein |
| MCC | mutated in colorectal cancers |
| MCF2 | MCF.2 cell line derived transforming sequence |
| MDM2 | mouse double minute 2, human homolog of; p53-binding protein |
| MEL | mel transforming oncogene (derived from cell line NK14)- RAB8 |
| | homolog |
| MEN1 | multiple endocrine neoplasia I |
| MET | met proto-oncogene (hepatocyte growth factor receptor) |
| MLH1 | mutL (E. coli) homolog 1 (colon cancer, nonpolyposis type 2) |
| MOS | v-mos Moloney murine sarcoma viral oncogene homolog |
| MPL | myeloproliferative leukemia virus oncogene |
| MSH2 | mutS (E. coli) homolog 2 (colon cancer, nonpolyposis type 1) |
| MYB | v-myb avian myeloblastosis viral oncogene homolog |
| MYBL1 | v-myb avian myeloblastosis viral oncogene homolog-like 1 |
| MYBL2 | v-myb avian myeloblastosis viral oncogene homolog-like 2 |
| MYC | v-myc avian myelocytomatosis viral oncogene homolog |
| MYCL1 | v-myc avian myelocytomatosis viral oncogene homolog 1, lung |
| | carcinoma derived |
| MYCN | v-myc avian myelocytomatosis viral related oncogene, |
| | neuroblastoma derived |
| NBL1 | neuroblastoma, suppression of tumorigenicity 1 |
| NF1 | neurofibromin 1 (neurofibromatosis, von Recklinghausen disease, |
| | Watson disease) |
| NF2 | neurofibromin 2 (bilateral acoustic neuroma) |
| NFKB2 | nuclear factor of kappa light polypeptide gene enhancer in B-cells 2 |
| | (p49/p100) |
| NKTR | natural killer-tumor recognition sequence |
| NOV | nephroblastoma overexpressed gene |
| NRAS | neuroblastoma RAS viral (v-ras) oncogene homolog |
| NTRK1 | Neurotrophic tyrosine kinase, receptor, type 1 |
| PACE | paired basic amino acid cleaving enzyme (furin, membrane |
| | associated receptor protein) |
| PDGFB | platelet-derived growth factor beta polypeptide (simian sarcoma viral |
| | (v-sis) oncogene homolog) |
| PIM1 | pim-1 oncogene |
| PTCH | patched (Drosophila) homolog |
| PVT1 | pvt-1 (murine) oncogene homolog, MYC activator |
| RAF1 | v-raf-1 murine leukemia viral oncogene homolog 1 |
| RALA | v-ral simian leukemia viral oncogene homolog A (ras related) |
| RALB | v-ral simian leukemia viral oncogene homolog B (ras related; GTP |
| | binding protein) |
| RB1 | retinoblastoma 1 (including osteosarcoma) |
| REL | v-rel avian reticuloendotheliosis viral oncogene homolog |
| RET | ret proto-oncogene (multiple endocrine neoplasia MEN2A, MEN2B |
| | and medullary thyroid carcinoma 1, Hirschsprung disease) |
| ROS1 | v-ros avian UR2 sarcoma virus oncogene homolog 1 |
| SKI | v-ski avian sarcoma viral oncogene homolog |
| SMARCB1 | SWI/SNF related, matrix associated, actin dependent regulator of |
| | chromatin, subfamily b, member 1 |
| SPI1 | spleen focus forming virus (SFFV) proviral integration oncogene spi1 |
| SPINK1 | serine protease inhibitor, Kazal type 1 |
| SRC | v-src avian sarcoma (Schmidt-Ruppin A-2) viral oncogene homolog |
| ST5 | Suppression of tumorigenicity 5 |
| SUPT3H | suppressor of Ty (S. cerevisiae) 3 homolog |
| SUPT5H | suppressor of Ty (S. cerevisiae) 5 homolog |
| SUPT6H | suppressor of Ty (S. cerevisiae) 6 homolog |
| TAL1 | T-cell acute lymphocytic leukemia 1 |
| TGFBR2 | Transforming growth factor, beta receptor II (70-80 kD) |
| THPO | thrombopoietin (myeloproliferative leukemia virus oncogene ligand, |
| | megakaryocyte growth and development factor) |
| THRA | thyroid hormone receptor, alpha (avian erythroblastic leukemia viral |
| | (v-erb-a) oncogene homolog) |
| THRB | thyroid hormone receptor, beta (avian erythroblastic leukemia viral |
| | (v-erb-a) oncogene homolog 2) |
| TIAM1 | T-cell lymphoma invasion and metastasis 1 |
| TIM | Oncogene TIM |
| TM4SF1 | transmembrane 4 superfamily member 1 |
| TNF | tumor necrosis factor (TNF superfamily, member 2) |
| TP53 | tumor protein p53 (Li-Fraumeni syndrome) |
| TP53BP2 | tumor protein p53-binding protein, 2 |
| TP73 | tumor protein p73 |
| VAV1 | vav 1 oncogene |
| VAV2 | vav 2 oncogene |
| VHL | von Hippel-Lindau syndrome |
| WNT1 | wingless-type MMTV integration site family, member 1 |
| WNT2 | wingless-type MMTV integration site family member 2 |
| WNT5A | wingless-type MMTV integration site family, member 5A |
| WT1 | Wilms tumor 1 |
| YES1 | v-yes-1 Yamaguchi sarcoma viral oncogene homolog 1 |

In another embodiment the present invention can be used for diagnosis of cancer by detection of target polynucleotide selected from the group consisting of DNA and RNA such as DNA derived from one or more tumor antigens such as TFPI2, NDRG4, GATA4 or GATA5 [2]. Such cancer is characterized by one or more mutations in one or more genes or genes selected from the group consisting of TFPI2, NDRG4, GATA4 or GATA5.

In another embodiment the present invention can be used for diagnosis of cancer by detection of target polynucleotide selected from the group consisting of DNA and RNA such as DNA derived from one or more tumor antigens such as RASSF2 and SFRP2 [2]. Such cancer is characterized by one or more mutations in one or more genes or genes selected from the group consisting of RASSF2 and SFRP2.

Diagnosis of Hereditary Diseases Other than Cancer

In another preferred embodiment the invention is used to diagnose one or more genetic, i.e. hereditary, diseases other than cancer, such one or more diseases selected from Table E herein below. In one embodiment wherein the method is used for the diagnosis of hereditary diseases other than cancer, the target polynucleotide is selected from the group consisting of DNA and RNA such as DNA derived from the individual to be diagnosed.

TABLE E
Diseases

CADASIL syndrome
Carboxylase Deficiency, Multiple, Late-Onset
Cerebelloretinal Angiomatosis, familial
Crohn's disease, fibrostenosing
Deficienc disease, Phenylalanine Hydroxylase
Fabry disease
Hereditary coproporphyria
Incontinentia pigmenti
Microcephaly
Polycystic kidney disease
Siderius X-linked mental retardation syndrome
caused by mutations in the PHF8 gene
achondroplasia

One specific embodiment thereof relates to the diagnosis of CADASIL syndrome. Another specific embodiment thereof relates to the diagnosis of Carboxylase Deficiency, Multiple, Late-Onset. Yet another specific embodiment thereof relates to the diagnosis of Cerebelloretinal Angiomatosis, familial. Yet another specific embodiment thereof relates to the diagnosis of Crohn's disease, fibrostenosing. Yet another specific embodiment thereof relates to the diagnosis of Deficiency disease, Phenylalanine Hydroxylase. Yet another specific embodiment thereof relates to the diagnosis of Fabry disease. Yet another specific embodiment thereof relates to the diagnosis of Hereditary coproporphyria. Yet another specific embodiment thereof relates to the diagnosis of Incontinentia pigmenti. Yet another specific embodiment thereof relates to the diagnosis of Microcephaly. Yet another specific embodiment thereof relates to the diagnosis of Polycystic kidney disease. Yet another specific embodiment thereof relates to the diagnosis of Siderius X-linked mental retardation syndrome caused by mutations in the PHF8 gene. Yet another specific embodiment thereof relates to the diagnosis of achondroplasia.

A further embodiment thereof relates to the detection of one or more disorder(s) selected from the group consisting of blood cell disorders, errors of amino acid metabolism, errors of organic acid metabolism, errors of fatty acid metabolism and miscellaneous multisystem diseases.

One specific embodiment relates to use of the method disclosed herein for detection of blood cell disorders such as e.g. Sickle cell anemia (Hb SS), Sickle-cell disease (Hb S/C), Hb S/Beta-Thalassemia (Hb S/Th), Variant hemoglobinopathies (including Hb E) and Glucose-6-phosphate dehydrogenase deficiency (G6PD).

Another specific embodiment relates to use of the method disclosed herein for detection of errors of amino acid metabolism, such as e.g. Tyrosinemia I (TYR I), Tyrosinemia II (TYR II), Tyrosinemia III (TYR III), Argininosuccinic aciduria (ASA), Citrullinemia (CIT), Citrullinemia type II (CIT II), Phenylketonuria (PKU), Maple syrup urine disease (MSUD), Homocystinuria (HCY), Benign hyperphenylalaninemia, Defects of biopterin cofactor biosynthesis, Defects of biopterin cofactor regeneration, and Hypermethioninemia.

Yet another specific embodiment relates to use of the method disclosed herein for detection of errors of organic acid metabolism, such as e.g. Glutaric acidemia type I (GA I), Hydroxymethylglutaryl lyase deficiency (HMG), Isovaleric acidemia (IVA), 3-Methylcrotonyl-CoA carboxylase deficiency (3MCC), Methylmalonyl-CoA mutase deficiency (MUT), Methylmalonic aciduria, cblA and cblB forms (MMA, Cbl A,B) and, Beta-ketothiolase deficiency (BKT), Propionic acidemia (PROP), Multiple-CoA carboxylase deficiency (MCD), Methylmalonic acidemia (Cbl C,D), Malonic acidemia, 2-Methyl 3-hydroxy butyric aciduria, Isobutyryl-CoA dehydrogenase deficiency, 2-Methylbutyryl-CoA dehydrogenase deficiency, 3-Methylglutaconyl-CoA hydratase deficiency, Glutaric acidemia type II, HHH syndrome (Hyperammonemia, hyperornithinemia, homocitrullinuria syndrome), Beta-methyl crotonyl carboxylase deficiency and Adenosylcobalamin synthesis defects.

Yet another specific embodiment relates to use of the method disclosed herein for detection of errors of fatty acid metabolism such as e.g. Long-chain hydroxyacyl-CoA dehydrogenase deficiency (LCHAD), Medium-chain acyl-CoA dehydrogenase deficiency (MCAD), Very-long-chain acyl-CoA dehydrogenase deficiency (VLCAD), Trifunctional protein deficiency (TFP), Carnitine uptake defect (CUD), Medium-chain ketoacyl-CoA thiolase deficiency, Dienoyl-CoA reductase deficiency, Glutaric acidemia type II, Carnitine palmityl transferase deficiency type 1, Carnitine palmityl transferase deficiency type 2, Short-chain acyl-CoA dehydrogenase deficiency (SCAD), Carnitine/acylcarnitine Translocase Deficiency (Translocase), Short-chain hydroxy Acyl-CoA dehydrogenase deficiency (SCHAD), Long-chain acyl-CoA dehydrogenase deficiency (LCAD) and Multiple acyl-CoA dehydrogenase deficiency (MADD);

Yet another specific embodiment relates to use of the method disclosed herein for detection of miscellaneous multisystem diseases such as e.g. Cystic fibrosis (CF), Congenital hypothyroidism (CH), Biotimidase deficiency (BIOT), Congenital adrenal hyperplasia (CAH), Classical galactosemia (GALT), Galactokinase deficiency and Galactose epimerase deficiency.

The diagnosis of hereditary diseases may be performed in a sample from a child or an adult, and in addition the diagnosis may be carried out on a foetal sample.

### Target Polynucleotide Prenata/Diagnosis

In an embodiment, the screening method of the present invention is used for prenatal diagnosis. Prenatal diagnosis or prenatal screening is the testing for diseases or conditions in a fetus or embryo, i.e. before the child is born. In this embodiment, the target polynucleotide is selected from the group consisting of DNA and RNA such as DNA. Accordingly, one embodiment of the invention relates to use of the method disclosed herein for the diagnosis of a foetus disorder.

In prenatal diagnosis, avoidance of false positive test results is essential. It is thus essential that no test results indicate that target polynucleotide is present in samples which are really devoid of said target polynucleotide. In this aspect the present invention is superior as compared to known methods for identification of target polynucleotide due to its superior specificity.

In prenatal diagnosis, the target polynucleotide may be obtained from any biological materiall available from the child, such as e.g. from a sample of tissue or body liquid obtained from a biopsy, from the amniotic fluid, from the placenta or from the blood of the mother. In one specific embodiment, the target polynucleotide is obtained from a sample of tissue or body liquid obtained from a biopsy, from the amniotic fluid or from the placenta. In another specific embodiment, the target polynucleotide is obtained from the blood of the mother.

One embodiment thereof relates to prenatal diagnosis or prenatal screening wherein birth defects are detected. One specific embodiment of the invention is directed to the detection of one or more birth defect(s) such as e.g. one or more birth defect(s) selected from the group consisting of neural tube defects, Down syndrome, chromosome abnormalities, genetic diseases and other conditions, such as spina bifida, cleft palate, Tay Sachs disease, sickle cell anemia, thalassemia, cystic fibrosis, and fragile x syndrome. One specific embodiment thereof relates to prenatal detection of neural tube defects. Another specific embodiment thereof relates to prenatal detection of Down syndrome. Yet another specific embodiment thereof relates to prenatal detection of chromosome abnormalities. Yet another specific embodiment thereof relates to prenatal detection of genetic diseases, such as e.g. one or more disorders selected from the group consisting of cystic fibrosis, trisomy 8, trisomy 9, trisomy 13, trisomy 18, trisomy 21, trisomy 22 or triple X syndrome. One specific and preferred embodiment thereof relates to the detection of trisomy 21. Another specific embodiment thereof relates to the detection of trisomy 13. Yet another specific embodiment thereof relates to the detection of trisomy 18. Another specific embodiment thereof relates to the detection of cystic fibrosis. Yet another specific embodiment thereof relates to prenatal detection of spina bifida. Yet another specific embodiment thereof relates to prenatal detection of cleft palate. Yet another specific embodiment thereof relates to prenatal detection of Tay Sachs disease. Yet another specific embodiment thereof relates to prenatal detection of sickle cell anemia. Yet another specific embodiment thereof relates to prenatal detection of thalassemia. Yet another specific embodiment thereof relates to prenatal detection of cystic fibrosis. Yet another specific embodiment thereof relates to prenatal detection of fragile x syndrome.

One further embodiment thereof relates to fetal screening with the purpose of determining characteristics generally not considered birth defects, such as e.g. for sex selection or the determination of the father of the child etc. One specific embodiment thereof relates to prenatal detection for determination of the sex of the child. Another specific embodiment thereof relates to prenatal determination of the father.

### Monitoring of Antisense Therapy

Antisense therapy is a form of treatment e.g. for genetic disorders or infections. When the genetic sequence of a particular gene is known to be causative of a particular disease, it is possible to synthesize a strand of nucleic acid that will inactivate it by effectively turning that gene “off” e.g. through binding to the messenger RNA (mRNA) produced by that gene. Accordingly, one embodiment of the invention relates to use of the method disclosed herein for the detection of polynucleotide used in antisense therapy. In an embodiment thereof, said polynucleotide is the target polynucleotide is made of nucleotides which are not known to occur naturally or a mixture of naturally occurring target polynucleotide and target polynucleotide made of nucleotides which are not known to occur naturally as defined herein. In one embodiment, said target polynucleotide comprises LNA-modified DNA, LNA-modified RNA and/or PNA.

In a specific embodiment, said target polynucleotide binds to the messenger RNA (mRNA) produced by the gene being causative of disease. In another specific embodiment, said target polynucleotide binds to a splicing site on pre-mRNA thus modifying the exon content of an mRNA.

In a particular embodiment, said antisence therapy is for use within treatment of one or more cancers as disclosed herein, diabetes, Amyotrophic lateral sclerosis (ALS), Duchenne muscular dystrophy, cytomegalovirus retinitis and diseases such as asthma and arthritis with an inflammatory component.

### Identification of Familial Relatives

In one embodiment, the method of the present invention is used for the identification of individuals by their respective DNA profiles.

In an embodiment wherein the method disclosed herein is used for the identification of familial relatives, the target polynucleotide detected is naturally occurring, such as one or more of DNA and/or RNA.

A specific embodiment relates to the use of the method according to the present invention for the identification of alledged relatives such as e.g. the identification of a mother or father of a child, such as wherein e.g. D21S11, D7S820, TH01, D13S317 and D19S433 are used as DNA markers.

In a particular embodiment thereof, the target polynucleotide may be obtained from any biological material available, such as e.g. from faeces, blood, semen, cerebrospinal fluid (CSF), sputum, vaginal fluid, urine, saliva, hair, other bodily fluids, tissue samples, whole organs, sweat, tears, skin cells, hair, bone, teeth or appropriate fluid or tissue from personal items (e.g. toothbrush, razor, etc.) or from samples (e.g. sperm or biopsy tissue or liquid) or other sub-structures of humans or animals.

### Personalized Medicine

Personalized medicine is a medical model emphasizing the systematic use of information about an individual patient to select or optimize that patient's preventative and therapeutic care. The present invention can be used with respect to personalized medicine. Detection of one or more specific target DNA sequences can e.g. be used to determine the drug and/or drug dose that should be applied.

### Type of Sample to be Analysed

The present invention relates to dected of target polynucleotide such as DNA derived from any sample such as a sample from a human or animal body. In another embodiment, the target nucleotide is derived from a human being, an animal, bacteria, virus, fungus, prions, protozoa and/or plant. In yet another embodiment, the target polynucleotide is isolate from a sample from a human or animal body. Sample sources include samples obtained from live as well as non-live sources, including but not limited to humans, animals, birds, insects, plants, algae, fungi's, yeast, viruses, bacteria and phages, multi-cellular and mono-cellular organisms.

Human and animal samples include faeces, blood, semen, cerebrospinal fluid (CSF), sputum, vaginal fluid, urine, saliva, hair, other bodily fluids, tissue samples, whole organs, sweat, tears, skin cells, hair, bone, teeth or appropriate fluid or tissue from personal items (e.g. toothbrush, razor, etc.) or from samples (e.g. sperm or biopsy tissue or liquid) or other sub-structures of humans or animals. The sample therefore may be a solid, semi-solid or a fluent sample.

Sources of pathogens include one or more bacteria, viruses, parasites and other infective organisms. Other sources may be environmental samples such as drinking water, sewage, or soil.

The sample to be isolated can be an invasive or non-invasive sample. Example invasive sampling include drawing of blood, resection of tissues, organs or part thereof (e.g. by biopsy) and drawing of cerebrospinal fluid (lumbar puncture). Examples of non-invasive sampling include collection of externally secreted fluids or material (e.g. sputum, urine, faeces).

The sample to be analysed can be treated in order to isolated, purify or enrich for the DNA.

### Individual to be Diagnosed or Tested

The individual to be diagnosed or tested can be a human being such as a man or a woman. The individual to be diagnosed can be a human being of any age, such as a foetus, an infant, a child or an adult.

A foetus to be diagnosed may be of any age such as from 8 to 40 week of gestagation, for example from 12 to 25 week of gestatation, such as from 16 to 20 week of gestagation. Any other individual to be diagnosed can be of any age such as from newborn to 120 years old, for example from 0 to 6 months, such as from 6 to 12 months, for example from 1 to 5 years, such as from 5 to 10 years, for example from 10 to 15 years, such as from 15 to 20 years, for example from 20 to 25 years, such as from 25 to 30 years, for example from 30 to 35 years, such as from 35 to 40 years, for example from 40 to 45 years, such as from 45 to 50 years, for example from 50 to 60 years, such as from 60 to 70 years, for example from 70 to 80 years, such as from 80 to 90 years, for example from 90 to 100 years, such as from 100 to 110 years, for example from 110 to 120 years.

The individual to be diagnosed and/or treated can be of any race such as a Caucasian, a black person, an East Asian person, a person of Mongoloid race, a person of Ethiopian race, a person of Negroid race, a person of American Indian race, or a person of Malayan race.

The individual to be diagnosed and/or treated can be healthy, ill, diagnosed with one or more disease(s), can have one or more symptoms of one or more diseases, can be asymptomatic or can be genetically disposed to one or more diseases.

The individual to be diagnosed can be selected from the group consisting of bacteria, vira, fungus, prions, protozoa and/or plants.

### Other Uses

In principle the present invention may be used in any method wherein detection of polynucleotide which may be made of naturally occurring nucleotides or which may be made of nucleotides which are not known to occur naturally or any mixture thereof, such as DNA and/or RNA, is relevant.

### Forensic Genetics

The method according to the present invention may be used for forensic genetics, including paternity testing or maternity testing. In an embodiment wherein the method disclosed herein is used within forensic genetics, the target polynucleotide is made of naturally occurring nucleotides.

Accordingly, one embodiment of the invention relates to the use of the method as disclosed herein within forensic science (often shortened to forensics). The method may thus be used within a broad spectrum of sciences to answer questions of interest to a legal system. One particular embodiment thereof relates to the use of the method of the invention in relation to a crime or a civil action.

In one embodiment, the target polynucleotide may be provided by the victim and/or the criminal. In one specific embodiment, the target polynucleotide is provided by the victim. In another specific embodiment, the target polynucleotide is provided by the criminal, such as by a suspected criminal.

One specific embodiment of the invention relates to the use of the method as disclosed herein within forensic pathology. Accordingly, said method may be used in the branch of pathology concerned with determining the cause of death by examination of a corpse.

One further specific embodiment of the invention relates to the use of the method as disclosed herein within forensic archaeology, such as e.g. for identification of buried small items or personal effects from a victim of crime; for identification of buried small items or personal effects from a criminal, and/or for recovery of any human remains such as e.g. buried in potential gravesites and/or mass graves.

One further specific embodiment of the invention relates to the use of the method as disclosed herein to assist forensic anthropology. Accordingly, an embodiment relates to the use of the method in the identification of remains, such as e.g. for the determination of particular characteristics such as e.g. race, sex, age and stature based on such remains.

One further specific embodiment of the invention relates to the use of the method as disclosed herein within forensic botany. Accordingly, an embodiment relates to the use of the method in order to gain information regarding possible crimes, such as e.g. from leaves, seeds and pollen found either on a body or at the scene of a crime.

One further specific embodiment of the invention relates to the use of the method as disclosed herein within rape investigation, e.g. for identification of the rapist.

One further specific embodiment of the invention relates to the use of the method as disclosed herein within paternity testing or maternity testing.

### Quantitative RNA Analysis

Another use of the present invention is analysis of gene expression in a human being or animal. In one embodiment the method disclosed herein can be used for quantitative RNA analysis, i.e. for quantification of target RNA.

### Detection of Microorganisms

The present invention can also be used for typing of microorganisms. Accordingly, the target DNA and/or RNA may be derived from one or more bacteria, one or more vira, one or more fungus, one or more prions, and/or one or more protozoa.

Furthermore, the method may be used to detect target polynucleotide such as DNA and/or RNA from bacteria, vira, fungus, prions, and/or protozoa found in a sample from the individual that is tested. Thus, in one embodiment the method may be used for detecting specific micro-organisms that may have caused infection, however the present invention may also be used for typing different serotypes of the same family of bacteria, such as different serotypes of the bacteria Streptococcus or the bacteria Salmonella, see also below.

Accordingly, one embodiment of the invention relates to the detection of an infection, which is e.g. the colonization of a host organism by parasite species. In a particular embodiment, said infection is caused by microscopic organisms or microparasites such as e.g. from one or more bacteria, one or more vira, one or more fungus, one or more prions, and/or one or more protozoa.

Diagnosis of infections can be difficult as specific signs and symptoms are rare. One embodiment of the invention relates to the detection of bacterial and viral infections that cause symptoms such as e.g. malaise, fever, and chills. In diagnostics, it is often difficult to distinguish the cause of a specific infection.

In an embodiment, the method of the invention may be use for diagnosis of one or more of the following group of diseases H. pylori, Methicillin-resistant Staphylococcus aureus, osteomyelitis, lyme disease, chlamydia, infection caused by virus and infections caused by bacteria.

One specific embodiment of the invention relates to the method disclosed herein for detection of H. pylori which is associated with inflammation of the stomach and is a common cause of stomach ulcers and gastritis.

One specific embodiment of the invention relates to the method disclosed herein for detection of m which predominantly affects the skin.

One specific embodiment of the invention relates to the method disclosed herein for detection of osteomyelitis which is a bone infection caused by various bacteria

One specific embodiment of the invention relates to the method disclosed herein for detection of lyme disease which is caused by at least three species of bacteria belonging to the genus Borrelia.

One specific embodiment of the invention relates to the method disclosed herein for detection of chlamydia which is a common sexually transmitted disease which can damage the female reproductive organs and result in permanent infertility.

One specific embodiment of the invention relates to the method disclosed herein for detection of infection caused by virus infections such as e.g. infections selected from the non-limiting group of: measles, hepatitis, herpes, infectious mononucleosis, HIV, hepatitis, herpes simplex, and, common to all mammals, endogenous retroviruses and Cytomegalovirus (CMV).

One specific embodiment of the invention relates to the method disclosed herein for detection of infections caused by bacteria.

In an embodiment, the method of the invention is used to analyse micro-organism contamination of a sample such as a feed, food, soil, drinking water etc., such as a sample of feed, food, drinking water etc.

In one embodiment the target DNA and/or RNA is derived from one or more bacteria, such as derived from one or more of the bacteria listed in Table F herein below.

TABLE F
Bacteria

Acetobacter aurantius, Acinetobacter species, Acinetobacter baumannii, Acinetobacter calcoaceticus,
Acinetobacter johnsonii, Acinetobacter junii, Acinetobacter lwoffii, Acinetobacter radioresistens,
Acinetobacter septicus, Acinetobacter schindleri, Acinetobacter ursingii; Actinomyces species: Actinomyces
bovis, Actinomyces bowdenii, Actinomyces canis, Actinomyces cardiffensis, Actinomyces catuli, Actinomyces
coleocanis, Actinomyces dentalis, Actinomyces denticolens, Actinomyces europaeus, Actinomyces funkei,
Actinomyces georgiae, Actinomyces gerencseriae, Actinomyces graevenitzii, Actinomyces hongkongensis,
Actinomyces hordeovulneris, Actinomyces howellii, Actinomyces humiferus, Actinomyces hyovaginalis,
Actinomyces israelii, Actinomyces marimammalium, Actinomyces meyeri, Actinomyces naeslundii, Actinomyces
nasicola, Actinomyces neuii, Actinomyces odontolyticus, Actinomyces oricola, Actinomyces radicidentis,
Actinomyces radingae, Actinomyces slackii, Actinomyces streptomycini, Actinomyces suimastitidis, Actinomyces
suis, Actinomyces turicensis, Actinomyces urogenitalis, Actinomyces vaccimaxillae, Actinomyces viscosus;
Actinobacillus species: Actinobacillus actinomycetemcomitans, Actinobacillus arthritidis, Actinobacillus capsulatus,
Actinobacillus delphinicola, Actinobacillus equuli, Actinobacillus hominis, Actinobacillus indolicus, Actinobacillus
lignieresii, Actinobacillus minor, Actinobacillus muris, Actinobacillus pleuropneumoniae, Actinobacillus porcinus,
Actinobacillus rossii, Actinobacillus scotiae, Actinobacillus seminis, Actinobacillus succinogenes, Actinobacillus
suis, Actinobacillus ureae; Aeromonas species: Aeromonas allosaccharophila, Aeromonas bestiarum, Aeromonas
bivalvium, Aeromonas encheleia, Aeromonas enteropelogenes, Aeromonas euchrenophila, Aeromonas hydrophila,
Aeromonas ichthiosmia, Aeromonas jandaei, Aeromonas media, Aeromonas molluscorum, Aeromonas popoffii,
Aeromonas punctata, Aeromonas salmonicida, Aeromonas schubertii, Aeromonas sharmana, Aeromonas simiae,
Aeromonas sobria, Aeromonas veronii; Afipia felis, Agrobacterium species: Agrobacterium radiobacter,
Agrobacterium rhizogenes, Agrobacterium rubi, Agrobacterium tumefaciens; Agromonas species, Alcaligenes
species: Alcaligenes aquatilis, Alcaligenes eutrophus, Alcaligenes faecalis, Alcaligenes latus, Alcaligenes
xylosoxidans; Alishewanella species, Alterococcus species, Anaplasma phagocytophilum, Anaplasma marginale,
Aquamonas species, Arcanobacterium haemolyticum, Aranicola species, Arsenophonus species, Azotivirga
species, Azotobacter vinelandii, Azotobacter chroococcum, Bacillary dysentery (Shigellosis), Bacillus species:
Bacillus abortus (Brucella melitensis biovar abortus), Bacillus anthracis (Anthrax), Bacillus brevis, Bacillus cereus,
Bacillus coagulans, Bacillus fusiformis, Bacillus globigii, Bacillus licheniformis, Bacillus megaterium, Bacillus
mycoides, Bacillus natto, Bacillus stearothermophilus, Bacillus subtilis, Bacillus sphaericus, Bacillus thuringiensis;
Bacteroides species: Bacteroides forsythus (Tannerella forsythensis), Bacteroides acidifaciens, Bacteroides
distasonis (reclassified as Parabacteroides distasonis), Bacteroides gingivalis, Bacteroides gracilis, Bacteroides
fragilis, Bacteroides oris, Bacteroides ovatus, Bacteroides putredinis, Bacteroides pyogenes, Bacteroides stercoris,
Bacteroides suis, Bacteroides tectus, Bacteroides thetaiotaomicron, Bacteroides vulgatus; Bartonella species:
Bartonella alsatica, Bartonella bacilliformis, Bartonella birtlesii, Bartonella bovis, Bartonella capreoli, Bartonella
clarridgeiae, Bartonella doshiae, Bartonella elizabethae, Bartonella grahamii, Bartonella henselae (cat scratch
fever), Bartonella koehlerae, Bartonella muris, Bartonella peromysci, Bartonella quintana, Bartonella rochalimae,
Bartonella schoenbuchii, Bartonella talpae, Bartonella taylorii, Bartonella tribocorum, Bartonella vinsonii spp.
Arupensis, Bartonella vinsonii spp. Berkhoffii, Bartonella vinsonii spp. Vinsonii, Bartonella washoensis; BCG
(Bacille Calmette- Guerin), Bergeyella zoohelcum (Weeksella zoohelcum), Bifidobacterium bifidum, Blastobacter
species, Blochmannia species, Bordetella species: ‘Bordetella ansorpii’, Bordetella avium, Bordetella bronchiseptica,
Bordetella hinzii, Bordetella holmesii, Bordetella parapertussis, Bordetella pertussis (Whooping cough), Bordetella
petrii, Bordetella trematum; Borrelia species, Borrelia burgdorferi, Borrelia afzelii, Borrelia anserina, Borrelia garinii,
Borrelia valaisiana, Borrelia hermsii, Borrelia Parkeri, Borrelia recurrentis; Bosea species, Bradyrhizobium species,
Brenneria species, Brucella species: Brucella abortus, Brucella canis, Brucella melitensis, Brucella neotomae,
Brucella ovis, Brucella suis, Brucella pinnipediae; Buchnera species, Budvicia species, Burkholderia species:
Burkholderia cepacia (Pseudomonas cepacia), Burkholderia mallei (Pseudomonas mallei/Actinobacillus mallei),
Burkholderia pseudomallei (Pseudomonas pseudomallei); Buttiauxella species, Calymmatobacterium granulomatis,
Campylobacter species: Campylobacter coli, Campylobacter concisus, Campylobacter curvus, Campylobacter
fetus, Campylobacter gracilis, Campylobacter helveticus, Campylobacter hominis, Campylobacter hyointestinalis,
Campylobacter insulaenigrae, Campylobacter jejuni, Campylobacter lanienae, Campylobacter lari, Campylobacter
mucosalis, Campylobacter rectus, Campylobacter showae, Campylobacter sputorum, Campylobacter upsaliensis;
Capnocytophaga canimorsus (Dysgonic fermenter type 2), Corynebacterium species, Cardiobacterium hominis,
Cedecea species, Chlamydia species: Chlamydia trachomatis (Lymphogranuloma venereum), Chlamydia
muridarum, Chlamydia suis; Chlamydophila species: Chlamydophila pneumoniae, Chlamydophila psittaci
(Psittacosis), Chlamydophila pecorum, Chlamydophila abortus, Chlamydophila felis, Chlamydophila caviae;
Citrobacter species: Citrobacter amalonaticus, Citrobacter braakii, Citrobacter farmeri, Citrobacter freundii,
Citrobacter gillenii, Citrobacter intermedius, Citrobacter koseri aka Citrobacter diversus, Citrobacter murliniae,
Citrobacter rodentium, Citrobacter sedlakii, Citrobacter werkmanii, Citrobacter youngae; Clostridium species:
Clostridium botulinum, Clostridium difficile, Clostridium novyi, Clostridium septicum, Clostridium tetani (Tetanus),
Clostridium welchii (Clostridium perfringens); Corynebacterium species: Corynebacterium diphtheriae (Diphtheria),
Corynebacterium amycolatum, Corynebacterium aquaticum, Corynebacterium bovis, Corynebacterium equi,
Corynebacterium flavescens, Corynebacterium glutamicum, Corynebacterium haemolyticum, Corynebacterium
jeikeiun (corynebacteria of group JK), Corynebacterium minutissimum (Erythrasma), Corynebacterium parvum
(also called Propionibacterium acnes), Corynebacterium pseudodiptheriticum (also called Corynebacterium
hofmannii), Corynebacterium pseudotuberculosis (also called Corynebacterium ovis), Corynebacterium pyogenes,
Corynebacterium urealyticum (corynebacteria of group D2), Corynebacterium renale, Corynebacterium striatum,
Corynebacterium tenuis (Trichomycosis palmellina, Trichomycosis axillaris), Corynebacterium ulcerans,
Corynebacterium xerosis; Coxiella burnetii (Q fever), Cronobacter species: Cronobacter sakazakii, Cronobacter
malonaticus, Cronobacter turicensis, Cronobacter muytjensii, Cronobacter dublinensis; Delftia acidovorans
(Comamonas acidovorans), Dickeya species, Edwardsiella species, Eikenella corrodens, Enterobacter species:
Enterobacter aerogenes, Enterobacter cloacae, Enterobacter sakazakii; Enterococcus species: Enterococcus
avium, Enterococcus durans, Enterococcus faecalis (Streptococcus faecalis/Streptococcus Group D),
Enterococcus faecium, Enterococcus solitarius, Enterococcus galllinarum, Enterococcus maloratus; Ehrlichia
chaffeensis, Erysipelothrix rhusiopathiae, Erwinia species, Escherichia species: Escherichia adecarboxylata,
Escherichia albertii, Escherichia blattae, Escherichia coli, Escherichia fergusonii, Escherichia hermannii,
Escherichia vulneris; Ewingella species, Flavobacterium species: Flavobacterium aquatile, Flavobacterium
branchiophilum, Flavobacterium columnare, Flavobacterium flevense, Flavobacterium gondwanense,
Flavobacterium hydatis, Flavobacterium johnsoniae, Flavobacterium pectinovorum, Flavobacterium psychrophilum,
Flavobacterium saccharophilum, Flavobacterium salegens, Flavobacterium scophthalmum, Flavobacterium
succinans; Francisella tularensis (Tularaemia), Francisella novicida, Francisella philomiragia, Fusobacterium
species: Fusobacterium necrophorum (Lemierre syndrome/Sphaerophorus necrophorus), Fusobacterium
nucleatum, Fusobacterium polymorphum, Fusobacterium novum, Fusobacterium mortiferum, Fusobacterium
varium; Gardnerella vaginalis, Gemella haemolysans, Gemella morbillorum (Streptococcus morbillorum),
Grimontella species, Haemophilus species: Haemophilus aegyptius (Koch-Weeks bacillus), Haemophilus
aphrophilus, Haemophilus avium, Haemophilus ducreyi (Chancroid), Haemophilus felis, Haemophilus haemolyticus,
Haemophilus influenzae (Pfeiffer bacillus), Haemophilus paracuniculus, Haemophilus parahaemolyticus,
Haemophilus parainfluenzae, Haemophilus paraphrophilus (Aggregatibacter aphrophilus), Haemophilus pertussis,
Haemophilus pittmaniae, Haemophilus somnus, Haemophilus vaginalis; Hafnia species, Hafnia alvei, Helicobacter
species: Helicobacter acinonychis, Helicobacter anseris, Helicobacter aurati, Helicobacter bilis, Helicobacter
bizzozeronii, Helicobacter brantae, Helicobacter Canadensis, Helicobacter canis, Helicobacter cholecystus,
Helicobacter cinaedi, Helicobacter cynogastricus, Helicobacter felis, Helicobacter fennelliae, Helicobacter ganmani,
Helicobacter heilmannii (Gastrospirillum hominis), Helicobacter hepaticus, Helicobacter mesocricetorum,
Helicobacter marmotae, Helicobacter muridarum, Helicobacter mustelae, Helicobacter pametensis, Helicobacter
pullorum, Helicobacter pylori (stomach ulcer), Helicobacter rappini, Helicobacter rodentium, Helicobacter
salomonis, Helicobacter trogontum, Helicobacter typhlonius, Helicobacter winghamensis; Human granulocytic
ehrlichiosis (Anaplasma phagocytophilum/Ehrlichia phagocytophila), Human monocytotropic ehrlichiosis
(Monocytic ehrlichiosis/Ehrlichia chaffeensis), Klebsiella species: Klebsiella granulomatis (Calymmatobacterium
granulomatis), Klebsiella mobilis, Klebsiella ornithinolytica, Klebsiella oxytoca, Klebsiella ozaenae, Klebsiella
planticola, Klebsiella pneumoniae, Klebsiella rhinoscleromatis, Klebsiella singaporensis, Klebsiella terrigena,
Klebsiella trevisanii, Klebsiella variicola; Kingella kingae, Kluyvera species, Lactobacillus species: Lactobacillus
acetotolerans, Lactobacillus acidifarinae, Lactobacillus acidipiscis, Lactobacillus acidophilus (Doderlein bacillus),
Lactobacillus agilis, Lactobacillus algidus, Lactobacillus alimentarius, Lactobacillus amylolyticus, Lactobacillus
amylophilus, Lactobacillus amylotrophicus, Lactobacillus amylovorus, Lactobacillus animalis, Lactobacillus antri,
Lactobacillus apodemi, Lactobacillus aviarius, Lactobacillus bifermentans, Lactobacillus brevis, Lactobacillus
buchneri, Lactobacillus camelliae, Lactobacillus casei, Lactobacillus catenaformis, Lactobacillus ceti, Lactobacillus
coleohominis, Lactobacillus collinoides, Lactobacillus composti, Lactobacillus concavus, Lactobacillus coryniformis,
Lactobacillus crispatus, Lactobacillus crustorum, Lactobacillus curvatus, Lactobacillus delbrueckii, Lactobacillus
delbrueckii subsp. Bulgaricus, Lactobacillus delbrueckii subsp. Lactis, Lactobacillus diolivorans, Lactobacillus
equi, Lactobacillus equigenerosi, Lactobacillus farraginis, Lactobacillus farciminis, Lactobacillus fermentum,
Lactobacillus fornicalis, Lactobacillus fructivorans, Lactobacillus frumenti, Lactobacillus fuchuensis, Lactobacillus
gallinarum, Lactobacillus gasseri, Lactobacillus gastricus, Lactobacillus ghanensis, Lactobacillus graminis,
Lactobacillus hammesii, Lactobacillus hamsteri, Lactobacillus harbinensis, Lactobacillus hayakitensis, Lactobacillus
helveticus, Lactobacillus hilgardii, Lactobacillus homohiochii, Lactobacillus iners, Lactobacillus ingluviei, Lactobacillus
intestinalis, Lactobacillus jensenii, Lactobacillus johnsonii, Lactobacillus kalixensis, Lactobacillus kefiranofaciens,
Lactobacillus kefiri, Lactobacillus kimchii, Lactobacillus kitasatonis, Lactobacillus kunkeei, Lactobacillus leichmannii,
Lactobacillus lindneri, Lactobacillus malefermentans, Lactobacillus mali, Lactobacillus manihotivorans, Lactobacillus
mindensis, Lactobacillus mucosae, Lactobacillus murinus, Lactobacillus nagelii, Lactobacillus namurensis,
Lactobacillus nantensis, Lactobacillus oligofermentans, Lactobacillus oris, Lactobacillus panis, Lactobacillus
pantheris, Lactobacillus parabrevis, Lactobacillus parabuchneri, Lactobacillus paracollinoides, Lactobacillus
parafarraginis, Lactobacillus parakefiri, Lactobacillus paralimentarius, Lactobacillus paraplantarum, Lactobacillus
pentosus, Lactobacillus perolens, Lactobacillus plantarum, Lactobacillus pontis, Lactobacillus psittaci, Lactobacillus
rennini, Lactobacillus reuteri, Lactobacillus rhamnosus, Lactobacillus rimae, Lactobacillus rogosae, Lactobacillus
rossiae, Lactobacillus ruminis, Lactobacillus saerimneri, Lactobacillus sakei, Lactobacillus salivarius, Lactobacillus
sanfranciscensis, Lactobacillus satsumensis, Lactobacillus secaliphilus, Lactobacillus sharpeae, Lactobacillus
siliginis, Lactobacillus spicheri, Lactobacillus suebicus, Lactobacillus thailandensis, Lactobacillus ultunensis,
Lactobacillus vaccinostercus, Lactobacillus vaginalis, Lactobacillus versmoldensis, Lactobacillus vini, Lactobacillus
vitulinus, Lactobacillus zeae, Lactobacillus zymae; Leclercia species, Legionella species: Legionella adelaidensis,
Legionella anisa, Legionella beliardensis, Legionella birminghamensis, Legionella bozemanii, Legionella brunensis,
Legionella busanensis, Legionella cherrii, Legionella cincinnatiensis, Legionella donaldsonii, Legionella drancourtii,
Legionella drozanskii, Legionella erythra, Legionella fairfieldensis, Legionella fallonii, Legionella feeleii, Legionella
geestiana, Legionella genomospecies, Legionella gratiana, Legionella gresilensis, Legionella hackeliae, Legionella
impletisoli, Legionella israelensis, Legionella jamestowniensis, ‘Candidatus Legionella jeonii’, Legionella jordanis,
Legionella lansingensis, Legionella londiniensis, Legionella longbeachae, Legionella lytica, Legionella maceachernii,
Legionella micdadei, Legionella moravica, Legionella nautarum, Legionella oakridgensis, Legionella parisiensis,
Legionella pneumophila, Legionella quateirensis, Legionella quinlivanii, Legionella rowbothamii, Legionella
rubrilucens, Legionella sainthelensi, Legionella santicrucis, Legionella shakespearei, Legionella spiritensis,
Legionella steigerwaltii, Legionella taurinensis, Legionella tucsonensis, Legionella wadsworthii, Legionella waltersii,
Legionella worsleiensis, Legionella yabuuchiae; Leminorella species, Leptospira species: Leptospira interrogans,
Leptospira kirschneri, Leptospira noguchii, Leptospira alexanderi, Leptospira weilii, Leptospira genomospecies 1,
Leptospira borgpetersenii, Leptospira santarosai, Leptospira inadai, Leptospira fainei, Leptospira broomii, Leptospira
licerasiae, Leptospira biflexa, Leptospira meyeri, Leptospira wolbachii, Leptospira genomospecies 3, Leptospira
genomospecies 4, Leptospira genomospecies 5; Lepromatous leprosy (Danielssen-Boeck disease), Leptospira
canicola, Leptospira hebdomadis, Leptospirosis (Weil disease/Leptospira icterohaemorrhagiae/Leptospira
interrogans serovar icterohaemorrhagiae), Leptotrichia, Leuconostoc species: Leuconostoc carnosum, Leuconostoc
citreum, Leuconostoc durionis, Leuconostoc fallax, Leuconostoc ficulneum, Leuconostoc fructosum, Leuconostoc
garlicum, Leuconostoc gasicomitatum, Leuconostoc gelidum, Leuconostoc inhae, Leuconostoc kimchii, Leuconostoc
lactis, Leuconostoc mesenteroides, Leuconostoc pseudoficulneum, Leuconostoc pseudomesenteroides; Listeria
species: Listeria grayi, Listeria innocua, Listeria ivanovii, Listeria monocytogenes (Listeriosis), Listeria seeligeri,
Listeria welshimeri; Methanobacterium extroquens, Microbacterium multiforme, Micrococcus species: Micrococcus
antarcticus, Micrococcus flavus, Micrococcus luteus, Micrococcus lylae, Micrococcus mucilaginosis, Micrococcus
roseus, Micrococcus sedentarius; Mobiluncus, Moellerella species, Morganella species, Moraxella species:
Moraxella atlantae, Moraxella boevrei, Moraxella bovis, Moraxella canis, Moraxella caprae, Moraxella catarrhalis
(Branhamella catarrhalis), Moraxella caviae, Moraxella cuniculi, Moraxella equi, Moraxella lacunata, Moraxella
lincolnii, Moraxella nonliquefaciens, Moraxella oblonga, Moraxella osloensis, Moraxella saccharolytica; Morganella
morganii, Mycobacterium species: Mycobacterium abscessus, Mycobacterium africanum, Mycobacterium agri,
Mycobacterium aichiense, Mycobacterium alvei, Mycobacterium arupense, Mycobacterium asiaticum, Mycobacterium
aubagnense, Mycobacterium aurum, Mycobacterium austroafricanum, Mycobacterium avium (Battey disease/
Lady Windermere syndrome), Mycobacterium avium paratuberculosis (implicated in Crohn's disease in humans
and Johne's disease in sheep), Mycobacterium avium silvaticum, Mycobacterium avium “hominissuis”,
Mycobacterium colombiense, Mycobacterium boenickei, Mycobacterium bohemicum, Mycobacterium bolletii,
Mycobacterium botniense, Mycobacterium bovis (Bovine tuberculosis), Mycobacterium branderi, Mycobacterium
brisbanense, Mycobacterium brumae, Mycobacterium canariasense, Mycobacterium caprae, Mycobacterium
celatum, Mycobacterium chelonae, Mycobacterium chimaera, Mycobacterium chitae, Mycobacterium chlorophenolicum,
Mycobacterium chubuense, Mycobacterium conceptionense, Mycobacterium confluentis, Mycobacterium
conspicuum, Mycobacterium cookii, Mycobacterium cosmeticum, Mycobacterium diernhoferi, Mycobacterium
doricum, Mycobacterium duvalii, Mycobacterium elephantis, Mycobacterium fallax, Mycobacterium farcinogenes,
Mycobacterium flavescens, Mycobacterium florentinum, Mycobacterium fluoroanthenivorans, Mycobacterium
fortuitum, Mycobacterium fortuitum subsp. Acetamidolyticum, Mycobacterium frederiksbergense, Mycobacterium
gadium, Mycobacterium gastri, Mycobacterium genavense, Mycobacterium gilvum, Mycobacterium goodii,
Mycobacterium gordonae (Mycobacterium aquae), Mycobacterium haemophilum, Mycobacterium hassiacum,
Mycobacterium heckeshornense, Mycobacterium heidelbergense, Mycobacterium hiberniae, Mycobacterium
hodleri, Mycobacterium holsaticum, Mycobacterium houstonense, Mycobacterium immunogenum, Mycobacterium
interjectum, Mycobacterium intermedium, Mycobacterium intracellulare, Mycobacterium kansasii, Mycobacterium
komossense, Mycobacterium kubicae, Mycobacterium kumamotonense, Mycobacterium lacus, Mycobacterium
lentiflavum, Mycobacterium leprae (causes leprosy or Hansen disease/Hanseniasis), Mycobacterium
lepraemurium, Mycobacterium madagascariense, Mycobacterium mageritense, Mycobacterium malmoense,
Mycobacterium marinum (Fish tank granuloma), Mycobacterium massiliense, Mycobacterium microti,
Mycobacterium monacense, Mycobacterium montefiorense, Mycobacterium moriokaense, Mycobacterium
mucogenicum, Mycobacterium murale, Mycobacterium nebraskense, Mycobacterium neoaurum, Mycobacterium
neworleansense, Mycobacterium nonchromogenicum, Mycobacterium novocastrense, Mycobacterium obuense,
Mycobacterium palustre, Mycobacterium parafortuitum, Mycobacterium parascrofulaceum, Mycobacterium
parmense, Mycobacterium peregrinum, Mycobacterium phlei, Mycobacterium phocaicum, Mycobacterium
pinnipedii, Mycobacterium porcinum, Mycobacterium poriferae, Mycobacterium pseudoshottsii, Mycobacterium
pulveris, Mycobacterium psychrotolerans, Mycobacterium pyrenivorans, Mycobacterium rhodesiae, Mycobacterium
saskatchewanense, Mycobacterium scrofulaceum, Mycobacterium senegalense, Mycobacterium seoulense,
Mycobacterium septicum, Mycobacterium shimoidei, Mycobacterium shottsii, Mycobacterium simiae, Mycobacterium
smegmatis, Mycobacterium sphagni, Mycobacterium szulgai, Mycobacterium terrae, Mycobacterium
thermoresistibile, Mycobacterium tokaiense, Mycobacterium triplex, Mycobacterium triviale, Mycobacterium
tuberculosis (major cause of human tuberculosis), Mycobacterium bovis, Mycobacterium africanum, Mycobacterium
canetti, Mycobacterium caprae, Mycobacterium pinnipedii′, Mycobacterium tusciae, Mycobacterium ulcerans
(causes Bairnsdale ulcer/Buruli ulcer), Mycobacterium vaccae, Mycobacterium vanbaalenii, Mycobacterium
wolinskyi, Mycobacterium xenopi; Mycoplasma species: Mycoplasma fermentans, Mycoplasma genitalium,
Mycoplasma hominis, Mycoplasma penetrans, Mycoplasma phocacerebrale, Mycoplasma pneumoniae,
Nanukayami (Seven-day fever/Gikiyami), Neisseria species: Neisseria gonorrhoea (Gonococcus/Gonorrhea),
Neisseria meningiditis (Meningococcus), Neisseria sicca, Neisseria cinerea, Neisseria elongata, Neisseria
flavescens, Neisseria lactamica, Neisseria mucosa, Neisseria polysaccharea, Neisseria subflava; Nitrobacter
species, Nocardia species: Nocardia asteroides, Nocardia brasiliensis, Nocardia caviae; Noma (cancrum oris/
gangrenous stomatitis), Obesumbacterium, Oligotropha species, Orientia tsutsugamushi (Scrub typhus),
Oxalobacter formigenes, Pantoea species: Pantoea agglomerans, Pantoea ananatis, Pantoea citrea, Pantoea
dispersa, Pantoea punctata, Pantoea stewartii, Pantoea terrea; Pasteurella species: Pasteurella aerogenes,
Pasteurella anatis, Pasteurella avium, Pasteurella bettyae, Pasteurella caballi, Pasteurella canis, Pasteurella
dagmatis, Pasteurella gallicida, Pasteurella gallinarum, Pasteurella granulomatis, Pasteurella langaaensis,
Pasteurella lymphangitidis, Pasteurella mairii, Pasteurella multocida, Pasteurella pneumotropica, Pasteurella
skyensis, Pasteurella stomatis, Pasteurella testudinis, Pasteurella trehalosi, Pasteurella tularensis, Pasteurella
ureae, Pasteurella volantium; Pediococcus species: Pediococcus acidilactici, Pediococcus cellicola, Pediococcus
claussenii, Pediococcus damnosus, Pediococcus dextrinicus, Pediococcus ethanolidurans, Pediococcus
inopinatus, Pediococcus parvulus, Pediococcus pentosaceus, Pediococcus stilesii; Peptostreptococcus
species: Peptostreptococcus anaerobius, Peptostreptococcus asaccharolyticus, Peptostreptococcus harei,
Peptostreptococcus hydrogenalis, Peptostreptococcus indoliticus, Peptostreptococcus ivorii, Peptostreptococcus
lacrimalis, Peptostreptococcus lactolyticus, Peptostreptococcus magnus, Peptostreptococcus micros,
Peptostreptococcus octavius, Peptostreptococcus prevotii, Peptostreptococcus tetradius, Peptostreptococcus
vaginalis; Photorhabdus species, Photorhizobium species, Plesiomonas shigelloides, Porphyromonas gingivalis,
Pragia species, Prevotella, Propionibacterium species: Propionibacterium acnes, Propionibacterium propionicus;
Proteus species: Proteus mirabilis, Proteus morganii, Proteus penneri, Proteus rettgeri, Proteus vulgaris;
Providencia species: Providencia friedericiana, Providencia stuartii; Pseudomonas species: Pseudomonas
aeruginosa, Pseudomonas alcaligenes, Pseudomonas anguilliseptica, Pseudomonas argentinensis, Pseudomonas
borbori, Pseudomonas citronellolis, Pseudomonas flavescens, Pseudomonas mendocina, Pseudomonas
nitroreducens, Pseudomonas oleovorans, Pseudomonas pseudoalcaligenes, Pseudomonas resinovorans,
Pseudomonas straminea, Pseudomonas aurantiaca, Pseudomonas aureofaciens, Pseudomonas chlororaphis,
Pseudomonas fragi, Pseudomonas lundensis, Pseudomonas taetrolens, Pseudomonas Antarctica, Pseudomonas
azotoformans, Pseudomonas brassicacearum, Pseudomonas brenneri, Pseudomonas cedrina, Pseudomonas
corrugate, Pseudomonas fluorescens, Pseudomonas gessardii, Pseudomonas libanensis, Pseudomonas
mandelii, Pseudomonas marginalis, Pseudomonas mediterranea, Pseudomonas meridiana, Pseudomonas
migulae, Pseudomonas mucidolens, Pseudomonas orientalis, Pseudomonas panacis, Pseudomonas proteolytica,
Pseudomonas rhodesiae, Pseudomonas synxantha, Pseudomonas thivervalensis, Pseudomonas tolaasii,
Pseudomonas veronii, Pseudomonas denitrificans, Pseudomonas pertucinogena, Pseudomonas cremoricolorata,
Pseudomonas fulva, Pseudomonas monteilii, Pseudomonas mosselii, Pseudomonas oryzihabitans, Pseudomonas
parafulva, Pseudomonas plecoglossicida, Pseudomonas putida, Pseudomonas balearica, Pseudomonas luteola,
Pseudomonas stutzeri, Pseudomonas amygdale, Pseudomonas avellanae, Pseudomonas caricapapayae,
Pseudomonas cichorii, Pseudomonas coronafaciens, Pseudomonas ficuserectae, Pseudomonas meliae,
Pseudomonas savastanoi, Pseudomonas syringae, Pseudomonas viridiflava, Pseudomonas abietaniphila,
Pseudomonas acidophila, Pseudomonas agarici, Pseudomonas alcaliphila, Pseudomonas alkanolytica,
Pseudomonas amyloderamosa, Pseudomonas asplenii, Pseudomonas azotifigens, Pseudomonas cannabina,
Pseudomonas coenobios, Pseudomonas congelans, Pseudomonas costantinii, Pseudomonas cruciviae,
Pseudomonas delhiensis, Pseudomonas excibis, Pseudomonas extremorientalis, Pseudomonas frederiksbergensis,
Pseudomonas fuscovaginae, Pseudomonas gelidicola, Pseudomonas grimontii, Pseudomonas indica, Pseudomonas
jessenii, Pseudomonas jinjuensis, Pseudomonas kilonensis, Pseudomonas knackmussii, Pseudomonas
koreensis, Pseudomonas lini, Pseudomonas lutea, Pseudomonas moraviensis, Pseudomonas otitidis,
Pseudomonas pachastrellae, Pseudomonas palleroniana, Pseudomonas papaveris, Pseudomonas peli,
Pseudomonas perolens, Pseudomonas poae, Pseudomonas pohangensis, Pseudomonas psychrophila,
Pseudomonas psychrotolerans, Pseudomonas rathonis, Pseudomonas reptilivora, Pseudomonas resiniphila,
Pseudomonas rhizosphaerae, Pseudomonas rubescens, Pseudomonas salomonii, Pseudomonas segitis,
Pseudomonas septica, Pseudomonas simiae, Pseudomonas suis, Pseudomonas thermotolerans, Pseudomonas
tremae, Pseudomonas trivialis, Pseudomonas turbinellae, Pseudomonas tuticorinensis, Pseudomonas
umsongensis, Pseudomonas vancouverensis, Pseudomonas vranovensis, Pseudomonas xanthomarina;
Rahnella species, Ralstonia species: Ralstonia basilensis, Ralstonia campinensis, Ralstonia eutropha, Ralstonia
gilardii, Ralstonia insidiosa, Ralstonia mannitolilytica, Ralstonia metallidurans, Ralstonia paucula, Ralstonia
pickettii, Ralstonia respiraculi, Ralstonia solanacearum, Ralstonia syzygii, Ralstonia taiwanensis; Raoultella
species, Rhodoblastus species, Rhodopseudomonas species, Rhinoscleroma, Rhizobium radiobacter,
Rhodococcus equi, Rickettsia species: Rickettsia africae, Rickettsia akari, Rickettsia australis, Rickettsia conorii,
Rickettsia felis, Rickettsia japonica, Rickettsia mooseri, Rickettsia prowazekii (Typhus fever), Rickettsia rickettsii,
Rickettsia siberica, Rickettsia typhi, Rickettsia conorii, Rickettsia africae, Rickettsia psittaci, Rickettsia quintana,
Rickettsia rickettsii, Rickettsia trachomae; Rothia dentocariosa, Salmonella species: Salmonella arizonae,
Salmonella Bongori, Salmonella enterica, Salmonella enteriditis, Salmonella paratyphi, Salmonella typhi (Typhoid
fever), Salmonella typhimurium, Salmonella salamae, Salmonella arizonae, Salmonella diarizonae, Salmonella
houtenae, Salmonella indica; Samsonia species, Serratia species: Serratia entomophila, Serratia ficaria, Serratia
fonticola, Serratia grimesii, Serratia liquefaciens, Serratia marcescens, Serratia odoriferae, Serratia plymuthica,
Serratia proteamaculans, Serratia quinivorans, Serratia rubidaea, Serratia ureilytica; Shewanella putrefaciens,
Shigella boydii, Shigella dysenteriae, Shigella flexneri, Shigella sonnei, Sodalis species, Spirillum species:
Spirillum minus rat bite fever, Staphylococcus species: Staphylococcus aureus, Staphylococcus auricularis,
Staphylococcus capitis, Staphylococcus caprae, Staphylococcus cohnii, Staphylococcus epidermidis, Staphylococcus
felis, Staphylococcus haemolyticus, Staphylococcus hominis, Staphylococcus intermedius, Staphylococcus
lugdunensis, Staphylococcus pettenkoferi, Staphylococcus saprophyticus, Staphylococcus schleiferi, Staphylococcus
simulans, Staphylococcus vitulus, Staphylococcus warneri, Staphylococcus xylosus; Stenotrophomonas species:
Stenotrophomonas acidaminiphila, Stenotrophomonas dokdonensis, Stenotrophomonas koreensis, Stenotrophomonas
maltophilia, Stenotrophomonas nitritireducens, Stenotrophomonas rhizophila; Streptobacillus species: Streptobacillus
moniliformis (Streptobacillary rat bite fever); Streptococcus species: Streptococcus Group A, Streptococcus Group B,
Streptococcus agalactiae, Streptococcus aginosus, Streptococcus avium, Streptococcus bovis, Streptococcus
canis, Streptococcus cricetus, Streptococcus faceium, Streptococcus faecalis, Streptococcus ferus, Streptococcus
gallinarum, Streptococcus lactis, Streptococcus milleri, Streptococcus mitior, Streptococcus mitis, Streptococcus
mutans, Streptococcus oralis, Streptococcus peroris, Streptococcus pneumoniae, Streptococcus pyogenes,
Streptococcus ratti, Streptococcus salivarius, Streptococcus sanguinis, Streptococcus sobrinus, Streptococcus
parasanguinis, Streptococcus suis, Streptococcus thermophilus, Streptococcus vestibularis, Streptococcus
viridans, Streptococcus uberis, Streptococcus zooepidemicus; Tatumella species, Trabulsiella species, Treponema
species: Treponema carateum (Pinta), Treponema denticola, Treponema endemicum (Bejel), Treponema pallidum
(Syphilis), Treponema pertenue (Yaws); Tropheryma whipplei (Whipple disease), Tuberculoid leprosy, Ureaplasma
urealyticum, Veillonella, Vibrio species: Vibrio aerogenes, Vibrio aestuarianus, Vibrio agarivorans, Vibrio albensis,
Vibrio alginolyticus, Vibrio brasiliensis, Vibrio calviensis, Vibrio campbellii, Vibrio chagasii, Vibrio cholerae
(Cholera), Vibrio cincinnatiensis, Vibrio Comma, Vibrio coralliilyticus, Vibrio crassostreae, Vibrio cyclitrophicus,
Vibrio diabolicus, Vibrio diazotrophicus, Vibrio ezurae, Vibrio fischeri, Vibrio fluvialis, Vibrio fortis, Vibrio furnissii,
Vibrio gallicus, Vibrio gazogenes, Vibrio gigantis, Vibrio halioticoli, Vibrio harveyi, Vibrio hepatarius, Vibrio
hispanicus, Vibrio ichthyoenteri, Vibrio kanaloae, Vibrio lentus, Vibrio litoralis, Vibrio logei, Vibrio mediterranei,
Vibrio metschnikovii, Vibrio mimicus, Vibrio mytili, Vibrio natriegens, Vibrio navarrensis, Vibrio neonatus, Vibrio
neptunius, Vibrio nereis, Vibrio nigripulchritudo, Vibrio ordalii, Vibrio orientalis, Vibrio pacinii, Vibrio parahaemolyticus,
Vibrio pectenicida, Vibrio penaeicida, Vibrio pomeroyi, Vibrio ponticus, Vibrio proteolyticus, Vibrio rotiferianus, Vibrio
ruber, Vibrio rumoiensis, Vibrio salmonicida, Vibrio scophthalmi, Vibrio splendidus, Vibrio superstes, Vibrio tapetis,
Vibrio tasmaniensis, Vibrio tubiashii, Vibrio vulnificus, Vibrio wodanis, Vibrio xuii; Vogesella indigofera, Wigglesworthia
species, Wolbachia secies, Xenorhabdus species, Yersinia enterocolitica, Yersinia pestis, Yersinia pseudotuberculosis,
and Yokenella species.

In one embodiment the target DNA and/or RNA is derived from one or more of the vira listed in Table G herein below. Accordingly, in an embodiment, the target polynucleotide is derived from a virus which is selected from the group of the vira listed in table G herein below.

TABLE G
Vira

Abelson murine leukemia virus (Ab-MLV, A-MuLV), acute laryngotracheobronchitis virus (or HPIV), Adelaide
River virus, Adeno-associated virus group (Dependevirus), Adenovirus, African horse sickness virus, African
swine fever virus, AIDS virus, Aleutian mink disease, parvovirus, alfalfa mosaic virus, Alphaherpesvirinae
(including HSV 1 and 2 and varicella), Alpharetrovirus (Avian leukosis virus, Rous sarcoma virus), Alphavirus,
alkhurma virus, ALV related virus, Amapari virus, Andean potato mottle virus, Aphthovirus, Aquareovirus,
arbovirus, arbovirus C, arbovirus group A, arbovirus group B, Arenavirus group, Argentine hemorrhagic fever
virus, Argentinian hemorrhagic fever virus, Arterivirus, Astrovirus, Ateline herpesvirus group, Aujezky's disease
virus, Aura virus, Ausduk disease virus, Australian bat lyssavirus, Aviadenovirus, avian erythroblastosis virus,
avian infectious bronchitis virus, avian leukemia virus, Avian leukosis virus (ALV), avian lymphomatosis virus,
avian myeloblastosis virus, avian paramyxovirus, avian pneumoencephalitis virus, avian reticuloendotheliosis
virus, avian sarcoma virus, avian type C retrovirus group, Avihepadnavirus, Avipoxvirus, B virus (Cercopithecine
herpesvirus 1), B19 virus (Parvovirus B19), Babanki virus, baboon herpesvirus, bacterial virus, baculovirus, barley
yellow dwarf virus, Barmah Forest virus, bean pod mottle virus, bean rugose mosaic virus, Bebaru virus, Berrimah
virus, Betaherpesvirinae, betaretrovirus, Bird flu, Birnavirus, Bittner virus, BK virus, Black Creek Canal virus,
bluetongue virus, Bolivian hemorrhagic fever virus, Boma disease virus, border disease of sheep virus, borna virus,
bovine alphaherpesvirus 1, bovine alphaherpesvirus 2, bovine coronavirus, bovine ephemeral fever virus, bovine
immunodeficiency virus, bovine leukemia virus, bovine leukosis virus, bovine mammillitis virus, bovine papillomavirus,
bovine papular stomatitis virus, bovine parvovirus, bovine syncytial virus, bovine type C oncovirus, bovine viral
diarrhea virus, bracovirus, broad bean mottle virus, broad bean stain virus, brome mosaic virus, Bromovirus, Buggy
Creek virus, bullet shaped virus group, Bunyamwera virus, Bunyavirus, Burkitt's lymphoma virus, Bwamba Fever,
Bwattany hetero virus, CA virus (Croup-associated virus/parainfluenza vius type 2), Calicivirus, California encephalitis
virus, camelpox virus, canarypox virus, canid herpesvirus, canine coronavirus, canine distemper virus, canine herpesvirus,
canine minute virus, canine parvovirus, Cano Delgadito virus, Capillovirus, caprine arthritis virus, caprine encephalitis
virus, Caprine Herpes Virus, Capripox virus, Cardiovirus, Carlavirus, Carmovirus, carrot mottle virus, Cassia yellow
blotch virus, Caulimovirus, Cauliflower mosaic virus, caviid herpesvirus 1, Cercopithecine herpesvirus 1, Cercopithecine
herpesvirus 2, cereal yellow dwarf virus, Chandipura virus, Changuinola virus, channel catfish virus, Charleville virus,
chickenpox virus, Chikungunya virus, chimpanzee herpesvirus, Chordopoxvirinae, chub reovirus, chum salmon virus,
Closterovirus, Cocal virus, Coho salmon reovirus, coital exanthema virus, Colorado tick fever virus, Coltivirus Columbia
SK virus, Commelina yellow mottle virus, Common cold virus, Comovirus, Condylomata accuminata, congenital
cytomegalovirus, contagious ecthyma virus, contagious pustular dermatitis virus, Coronavirus, Corriparta virus, coryza
virus, cowpea chlorotic mottle virus, cowpea mosaic virus, cowpea virus, cowpox virus, coxsackie virus, CPV (cytoplasmic
polyhedrosis virus), cricket paralysis virus, Crimean-Congo hemorrhagic fever virus, croup associated virus, Crypotovirus,
Cucumovirus, Cypovirus, Cytomegalovirus (HCMV or Human Herpesvirus 5 HHV-5), cytoplasmic polyhedrosis virus,
Cytorhabdovirus, deer papillomavirus, Deltaretrovirus (Human T-lymphotropic virus), Deformed wing virus DWV,
Dengue, Densovirus, Dependovirus, Dhori virus, Dianthovirus, diploma virus, DNA virus, Dobrava-Belgrade Virus, Dog
Flu, Drosophila C virus, duck hepatitis B virus, duck hepatitis virus 1, duck hepatitis virus 2, duovirus, Duvenhage virus,
eastern equine encephalitis virus, eastern equine encephalomyelitis virus, Ebola virus, Ebola-like virus, Echovirus,
echovirus 10, echovirus 28, echovirus 9, ectromelia virus, EEE virus (Eastern equine encephalitis virus), EIA virus
(equine infectious anemia), EMC virus (Encephalomyocarditis), Emiliania huxleyi virus 86, encephalitis virus,
encephalomyocarditis virus, Endogenous retrovirus, Enterovirus, Entomopoxvirinae, Entomopoxvirus A, Entomopoxvirus
B, Entomopoxvirus C, enzyme elevating virus, epidemic hemorrhagic fever virus, epizootic hemorrhagic disease virus,
Epsilonretrovirus, Epstein-Barr virus (EBV; Human herpesvirus 4 HHV-4), equid alphaherpesvirus 1, equid alphaherpesvirus
4, equid herpesvirus 2, equine abortion virus, equine arteritis virus, equine encephalosis virus, equine infectious anemia
virus, equine morbillivirus, equine rhinopneumonitis virus, equine rhinovirus, Eubenangu virus, European elk papillomavirus,
European swine fever virus, Everglades virus, Eyach virus, Fabavirus, felid herpesvirus 1, feline calicivirus, feline fibrosarcoma
virus, feline herpesvirus, feline immunodeficiency virus, feline infectious peritonitis virus, feline leukemia/sarcoma
virus, feline leukemia virus, feline panleukopenia virus, feline parvovirus, feline sarcoma virus, feline syncytial virus, Fijivirus,
Filovirus, Flanders virus, Flavivirus, foot and mouth disease virus, Fort Morgan virus, Four Corners hantavirus, fowl adenovirus
1, Fowlpox virus, Friend virus, Furovirus, Gammaherpesvirinae, gammaretrovirus, GB virus C(GBV-C; formerly Hepatitis
G virus), Geminivirus, German measles virus, Getah virus, gibbon ape leukemia virus, green monkey virus (mullburg),
glandular fever virus, goatpox virus, golden shinner virus, Gonometa virus, goose parvovirus, granulosis virus, Gross' virus,
ground squirrel hepatitis B virus, group A arbovirus, Guanarito virus, guinea pig cytomegalovirus, guinea pig type C virus,
Hantavirus, hard clam reovirus, hare fibroma virus, HCMV (human cytomegalovirus), helper virus, hemadsorption virus 2,
hemagglutinating virus of Japan, hemorrhagic fever virus, Hendra virus, Henipaviruses, Hepadnavirus, hepatitis A virus,
hepatitis B virus, hepatitis C virus, hepatitis D (delta) virus, hepatitis E virus, hepatitis F virus, hepatitis G virus, hepatitis
nonA nonB virus, hepatoencephalomyelitis reovirus 3, Hepatovirus, heron hepatitis B virus, herpes B virus, Herpes simplex
virus, herpes simplex virus 1, herpes simplex virus 2, Herpesvirus, Herpes zoster, Herpes virus 6, Herpes virus 7, Herpes
virus 8, Herpesvirus ateles, Herpesvirus hominis, Herpesvirus saimiri, Herpesvirus suis, Herpesvirus varicellae, Highlands
J virus, Hirame rhabdovirus, HIV-1, hog cholera virus, Hordeivirus, Horse Flu, HTLV-1, HTLV-2, human adenovirus 2,
human alphaherpesvirus 1, human alphaherpesvirus 2, human alphaherpesvirus 3, human B lymphotropic virus, human
betaherpesvirus 5, human coronavirus, Human enterovirus A, Human enterovirus B, Human Flu, human foamy virus,
human gammaherpesvirus 4, human gammaherpesvirus 6, human hepatitis A virus, human herpesvirus 1 group, human
herpesvirus 2 group, human herpesvirus 3 group, human herpesvirus 4 group, human herpesvirus 6, human herpesvirus
8, human immunodeficiency virus (HIV), human immunodeficiency virus 1, human immunodeficiency virus 2, Human
metapneumovirus, human papillomavirus, human T cell leukemia virus, human T cell leukemia virus I, human T cell
leukemia virus II, human T cell leukemia virus III, human T cell lymphoma virus I, human T cell lymphoma virus II, human
T cell lymphotropic virus type 1, human T cell lymphotropic virus type 2, human T lymphotropic virus I, human T lymphotropic
virus II, human T lymphotropic virus III, ichnovirus, llarvirus, infantile gastroenteritis virus, infectious bovine rhinotracheitis
virus, infectious haematopoietic necrosis virus, infectious pancreatic necrosis virus, influenza virus, influenzavirus A,
influenzavirus B, influenzavirus C, influenzavirus D, influenzavirus pr8, insect iridescent virus, insect virus, interfering
virus, iridovirus, Isavirus, Japanese B virus, Japanese encephalitis virus, JC virus, Junin virus, Johnson grass mosaic
virus, Kaposi's sarcoma-associated herpesvirus, Kemerovo virus, Kilham's rat virus, Klamath virus, Kolongo virus, Korean
hemorrhagic fever virus, kumba virus, Kumlinge virus, Kunjin virus, Kyasanur forest disease, Kyzylagach virus, La Crosse
virus, lactic dehydrogenase elevating virus, Lagos bat virus, Lambda phage, langat virus, Langur virus, lapine parvovirus,
Lassa fever virus, Lassa virus, latent rat virus, LCM virus, Leaky virus, Lentivirus, Leporipoxvirus, leukemia virus, leukovirus,
louping ill virus, lumpy skin disease virus, Luteovirus, lymphadenopathy associated virus, Lymphocytic choriomeningitis
virus (LCMV), Lymphocryptovirus, lymphocytic choriomeningitis virus, lymphoproliferative virus group, Lyssavirus, Machupo
virus, mad itch virus, maize chlorotic dwarf virus, maize rough dwarf virus, mammalian type B oncovirus group, mammalian
type B retroviruses, mammalian type C retrovirus group, mammalian type D retroviruses, mammary tumor virus, Mapuera
virus, Marafivirus, Marburg virus, Marburg-like virus, Mason Pfizer monkey virus, Mastadenovirus, Mayaro virus, ME virus,
Measles virus, Melandrium yellow fleck virus, Menangle virus, Mengo virus, Mengovirus, Merkel cell polyomavirus,
Middelburg virus, milkers nodule virus, mink enteritis virus, minute virus of mice, MLV related virus, MM virus, Mokola virus,
Molluscipoxvirus, Molluscum contagiosum virus, Moloney murine leukemia virus (M-MuLV), monkey B virus, Monkeypox
virus, Mononegavirales, Morbillivirus, Mount Elgon bat virus, mouse cytomegalovirus, mouse encephalomyelitis virus,
mouse hepatitis virus, mouse K virus, mouse leukemia virus, mouse mammary tumor virus, mouse minute virus, mouse
pneumonia virus, mouse poliomyelitis virus, mouse polyomavirus, mouse sarcoma virus, mousepox virus, Mozambique
virus, Mucambo virus, mucosal disease virus, Mumps virus, murid betaherpesvirus 1, murid cytomegalovirus 2, murine
cytomegalovirus group, murine encephalomyelitis virus, murine hepatitis virus, murine leukemia virus, murine nodule inducing
virus, murine polyomavirus, murine sarcoma virus, Muromegalovirus, Murray Valley encephalitis virus, myxoma virus,
Myxovirus, Myxovirus multiforme, Myxovirus parotitidis, Nairobi sheep disease virus, Nairovirus, Nanirnavirus, Nariva virus,
Ndumo virus, Necrovirus, Neethling virus, Nelson Bay virus, Nemtick Virus, Nepovirus, neurotropic virus, New World
Arenavirus, newborn pneumonitis virus, Newcastle disease virus, Nipah virus, noncytopathogenic virus, Norovirus, Norwalk
virus, nuclear polyhedrosis virus (NPV), nipple neck virus, O'nyong'nyong virus, oat sterile dwarf virus, Ockelbo virus,
Omsk hemorrhagic fever virus, oncogenic virus, oncogenic viruslike particle, oncornavirus, Orbivirus, Orf virus, Oropouche
virus, Orthohepadnavirus, orthomyxovirus, Orthopoxvirus, Orthoreovirus, Orungo, ovine papillomavirus, ovine catarrhal
fever virus, owl monkey herpesvirus, Palyam virus, Papillomavirus, Papillomavirus sylvilagi, Papovavirus, Parainfluenza
virus human (HPIV), parainfluenza virus type 1 human (HPIV-1), parainfluenza virus type 2 human (HPIV-2), parainfluenza
virus type 3 human (HPIV-3), parainfluenza virus type 4 human (HPIV-4), Paramyxovirus, Parapoxvirus, paravaccinia
virus, parsnip yellow fleck virus, Parvovirus, Parvovirus B19, pea enation mosaic virus, Pestivirus, Phlebovirus, phocine
distemper virus, Phytoreovirus, Picodnavirus, Picornavirus, pig cytomegalovirus, pigeonpox virus, Piry virus, Pixuna virus,
plant rhabdovirus group, plant virus, pneumonia virus of mice, Pneumovirus, Poliomyelitis virus, Poliovirus, Polydnavirus,
polyhedral virus, Polyoma virus, Polyomavirus, Polyomavirus bovis, Polyomavirus cercopitheci, Polyomavirus hominis
2, Polyomavirus maccacae 1, Polyomavirus muris 1, Polyomavirus muris 2, Polyomavirus papionis 1, Polyomavirus
papionis 2, Polyomavirus sylvilagi, Pongine herpesvirus 1, porcine epidemic diarrhea virus, porcine hemagglutinating
encephalomyelitis virus, porcine parvovirus, porcine transmissible gastroenteritis virus, porcine type C virus, Potato leaf
roll virus, Potato mop top virus, Potato virus Y, Potexvirus, Potyvirus, Powassan encephalitis virus, Poxvirus, poxvirus
variolae, Prospect Hill virus, provirus, pseudocowpox virus, pseudorabies virus, psittacinepox virus, Puumala virus, Qalyub
virus, Quail pea mosaic virus, quailpox virus, Queensland fruitfly virus, Quokkapox virus, rabbit fibroma virus, rabbit
kidney vacuolating virus, rabbit papillomavirus, Rabies virus, raccoon parvovirus, raccoonpox virus, radish mosaic virus,
Ranikhet virus, rat cytomegalovirus, rat parvovirus, rat virus, Rauscher's virus, recombinant vaccinia virus, recombinant
virus, Red Clover Necrotic Mosaic Virus, reovirus, reovirus 1, reovirus 2, reovirus 3, reptilian type C virus, Respiratory
syncytial virus, respiratory virus, reticuloendotheliosis virus, Retrovirus, Rhabdovirus, Rhabdovirus carpia, Rhadinovirus,
Rhinovirus, Rhizidiovirus, rice dwarf virus, rice gall dwarf virus, rice hoja blanca virus, rice ragged stunt virus, Rift Valley
fever virus, Riley's virus, rinderpest virus, RNA tumor virus, RNA virus, Roseolovirus, Ross River virus, Rotavirus, rougeole
virus, Rous sarcoma virus, Rubella virus, rubeola virus, Rubivirus, Russian autumn encephalitis virus, S6-14-03 virus,
SA 11 simian virus, SA 15, SA2 virus, SA6 virus, SA8 virus, Sabia virus, Sabio virus, Sabo virus, Saboya virus, Sabulodes
caberata GV, Sacbrood virus, Saccharomyces cerevisiae virus L-A, Saccharomyces cerevisiae virus La, Saccharomyces
cerevisiae virus LBC, Sagiyama virus, Saguaro cactus virus, Saimiriine herpesvirus 1, Saimiriine herpesvirus 2, Sainpaulia
leaf necrosis virus, SaintAbb's Head virus, Saint-Floris virus, Sakhalin virus, Sal Vieja virus, Salanga virus, Salangapox
virus, Salehabad virus, salivary gland virus, Salmonid herpesvirus 1, Salmonid herpesvirus 2, Salmonis virus, Sambucus
vein clearing virus, Samia cynthia NPV, Samia pryeri NPV, Samia ricini NPV, Sammons' Opuntia virus, SanAngelo virus,
San Juan virus, San Miguel sealion virus, San Perlita virus, Sand rat nuclear inclusion agents, Sandfly fever Naples virus,
Sandfly fever Sicilian virus, Sandjimba virus, Sango virus, Santa Rosa virus, Santarem virus, Santosai temperate virus,
Sapphire II virus, Sapporo-like virus, Saraca virus, Sarracenia purpurea virus, SARS virus, satellite virus, Sathuperi virus,
Satsuma dwarf virus, Saturnia pavonia virus, Saturnia pyri NPV, Saumarez Reef virus, Sawgrass virus, Sceliodes cordalis
NPV, Schefflera ringspot virus, Sciaphila duplex GV, Scirpophaga incertulas NPV, Sciurid herpesvirus, Sciurid herpesvirus
2, Scoliopteryx libatFix NPV, Scopelodes contracta NPV, Scopelodes venosa NPV, Scopula subpunctaria NPV, Scotogramma
trifolii GV, Scotogramma trifolu NPV, Scrophularia mottle virus, SDAV (sialodacryoadenitis virus), sealpox virus, Selenephera
lunigera NPV, Selepa celtis GV, Seletar virus, Selidosema suavis NPV, Semidonta biloba NPV, Semiothisa sexmaculata
GV, Semliki Forest Virus, Sena Madureira virus, Sendai virus, SENV-D, SENV-H, Seoul virus, Sepik virus, Serra do Navio
virus, Serrano golden mosaic virus, Sesame yellow mosaic virus, Sesamia calamistis NPV, Sesamia cretica GV, Sesamia
inferens NPV, Sesamia nonagrioides GV, Setora nitens virus, Shallot latent virus, Shamonda virus, Shark River virus,
Sheep associated malignant catarrhal fever, Sheep papillomavirus, Sheep pulmonary adenomatosis associated herpesvirus,
sheeppox virus, Shiant Islands virus, Shokwe virus, Shope fibroma virus, Shope papilloma virus, Shuni virus, Siamese
cobra herpesvirus, Sibine fusca densovirus, Sida golden mosaic virus (SiGMV), Sida golden yellow vein virus(SiGYW),
Sigma virus, Sikte water-borne virus, Silverwater virus, Simbu virus, Simian adenoviruses 1 to 27, Simian agent virus 12,
Simian enterovirus 1 to 18, simian foamy virus, Simian hemorrhagic fever virus, simian hepatitis A virus, simian human
immunodeficiency virus, simian immunodeficiency virus, simian parainfluenza virus, Simian rotavirus SA11, Simian
sarcoma virus, simian T cell lymphotrophic virus, Simian type D virus 1, Simian vancella herpesvirus, simian virus, simian
virus 40, Simplexvirus, Simulium vittatum densovirus, Sin Nombre virus, Sindbis virus, Sintlem's onion latent virus, Sixgun
city virus, Skunkpox virus, Smallpox virus, Smelt reovirus, Smerinthus ocellata NPV, Smithiantha virus, Snakehead
rhabdovirus, Snowshoe hare virus, Snyder-Theilen feline sarcoma virus, Sobemovirus, Sofyn virus, Soil-borne wheat
mosaic virus, Sokoluk virus, Solanum apical leaf curl virus, Solanum nodiflorum mottle virus, Solanurn yellows virus,
Soldado virus, Somerville virus 4, Sonchus mottle virus, Sonchus virus, Sonchus yellow net virus, Sorghum chlorotic
spot virus, Sorghum mosaic virus, Sorghum virus, Sororoca virus, Soursop yellow blotch virus, SouthAfrican passiflora
virus, South American hemorrhagic fever viruses, SouthAfrican passiflora virus, South River virus, Southern bean mosaic
virus, Southern potato latent virus, Sowbane mosaic virus, Sowthistle yellow vein virus, Soybean chlorotic mottle virus,
Soybean crinkle leaf virus, Soybean dwarf virus, Soybean mosaic virus, SPAr-2317 virus, Sparganothis pettitana NPV,
sparrowpox virus, Spartina mottle virus, Spectacled caimanpox virus, SPH 114202 virus, Sphenicid herpesvirus 1, Sphinx
ligustri NPV, Spider monkey herpesvirus, Spilarctia subcarnea NPV, Spilonota ocellana NPV, Spilosoma lubricipeda NPV,
Spinach latent virus, Spinach temperate virus, Spiroplasma phage 1, Spiroplasma phage 4, Spiroplasma phage aa,
Spiroplasma phage C1/TS2, Spodoptera exempta cypovirus, Spodoptera exigua virus, Spodoptera frugiperda virus,
Spodoptera latifascia virus, Spodoptera littoralis, Spodoptera mauritia virus, Spodoptera ornithogalli virus, Spondweni
virus, spring beauty latent virus, Spring viremia of carp virus, Spumavirus (SFV, HFV), Squash leaf curl virus, squash
mosaic virus, squirrel fibroma virus, Squirrel monkey herpesvirus, squirrel monkey retrovirus, SR-1 virus, Sri Lankan
passionfruit mottle virus, Sripur virus, SSV 1 virus group, StAbbs Head virus, St. Louis encephalitis virus, Staphylococcus
phage 07, Staphylococcus phage 187, Staphylococcus phage 2848A, Staphylococcus phage 3A, Staphylococcus phage
44A HJD, Staphylococcus phage 77, Staphylococcus phage B11-M15, Staphylococcus phage Twort, Starlingpox virus,
Statice virus Y, P, STLV (simian T lymphotropic virus) type I, STLV (simian T lymphotropic virus) type II, STLV (simian
T lymphotropic virus) type III, stomatitis papulosa virus, Stratford virus, Strawberry crinkle virus, Strawberry latent ringspot
virus, Strawberry mild yellow edge virus, Strawberry vein banding virus, Streptococcus phage 182, Streptococcus phage
2BV, Streptococcus phage A25, Streptococcus phage 24, Streptococcus phage PE1, Streptococcus phage VD13,
Streptococcus phage fD8, Streptococcus phage CP-1, Streptococcus phage Cvir, Streptococcus phage H39, Strigid
herpesvirus 1, Striped bass reovirus, Striped Jack nervous, necrosis virus, Stump-tailed macaque virus, submaxillary
virus, Subterranean clover mottle virus, Subterranean clover mottle virus satellite, Subterranean clover red leaf virus,
Subterranean clover stunt virus, Sugarcane bacilliform virus, Sugarcane mild mosaic virus, Sugarcane mosaic virus,
Sugarcane streak virus, suidalphaherpesvirus 1, suid herpesvirus 2, Suipoxvirus, Sulfolobus virus 1, Sunday Canyon
virus, Sunflower crinkle virus, Sunflower mosaic virus, Sunflower rugose mosaic virus, Sunflower yellow blotch virus,
Sunflower yellow ringspot virus, Sun-hemp mosaic virus, swamp fever virus, Sweet clover necrotic mosaic virus, Sweet
potato A virus, Sweet potato chlorotic leafspot virus, Sweet potato feathery mottle virus, Sweet potato internal cork virus,
Sweet potato latent virus, Sweet potato mild mottle virus, Sweet potato russet crack virus, Sweet potato vein mosaic
virus, Sweet potato yellow dwarf virus, Sweetwater Branch virus, Swine cytomegalovirus, Swine Flu, Swine infertility and
respiratory syndrome virus, swinepox virus, Swiss mouse leukemia virus, Sword bean distortion mosaic virus, Synaxis
jubararia NPV, Synaxis pallulata NPV, Synetaeris tenuifemur virus, Syngrapha selecta NPV, T4 phage, T7 phage, TAC
virus, Tacaiuma virus, Tacaribe complex virus, Tacaribe virus, Tadpole edema virus LT 1-4, Taggert virus, Tahyna
virus, Tai virus, Taiassui virus, Tamana bat virus, Tamarillo mosaic virus, Tamdy virus, [[Tamiami virus, Tanapox virus,
Tanga virus, Tanjong Rabok virus, Taro bacilliform virus, Badnavirus Tataguine virus, Taterapox virus, Taterapox virus,
Teasel mosaic virus, Tehran virus, Telfairia mosaic virus, Telok Forest virus, Tembe virus, Tembusu virus, Tench
reovirus, Tensaw virus, Tenvivirus, Tephrosia symptomless virus, Termeil virus, Tete virus, Tetralopha scortealis NPV,
Tetropium cinnamoptemm NPV, Texas pepper virus, Thailand virus, Thaumetopoea pityocampa virus, Theiler's
encephalomyelitis virus, Theiler's virus, Theophila mandarina NPV, Theretra japonica NPV, Thermoproteus virus 1,
Thermoproteus virus 2, Thermoproteus virus 3, Thermoproteus virus 4, Thiafora virus, Thimiri virus, Thistle mottle virus,
Thogoto virus, Thormodseyjarklettur virus, Thosea asigna virus, Thosea baibarana NPV, Thosea sinensis GV,
Thottapalayam virus, Thylidolpteryx ephemeraeformis NPV, Thymelicus lineola NPV, Tibrogargan virus, Ticera castanea
NPV, Tick borne encephalitis virus (TBEV) - European and Far Eastern subtypes, Tillamook virus, Tilligerry virus,
Timbo virus, Tilmboteua virus, Tilmaroo virus, Tindholmur virus, Tinea pellionella NPV, Tineola hisselliella NPV, Tinpula
paludosa NPV, Tinracola plagiata NPV, Tioman virus, Tlacotalpan virus, Tobacco bushy top virus, Tobacco etch virus,
Tobacco leaf curl virus, Tobacco mild green mosaic virus, tobacco mosaic virus, Tobacco mosaic virus satellite, Tobacco
mottle virus, Tobacco necrosis virus, Tobacco necrosis virus satellite, Tobacco necrosis virus small satellite, Tobacco
necrotic dwarf virus, tobacco rattle virus, Tobacco ringspot virus, Tobacco streak virus, Tobacco stunt virus, Tobacco
vein banding mosaic virus, Tobacco vein distorting virus Tobacco vein mottling virus, Tobacco wilt virus, Tobacco yellow
dwarf virus, Tobacco yellow net virus, Tobacco yellow vein virus, Tobamovirus Tobravirus, Togavirus, Tomato apical
stunt viroid, Tomato aspermy virus, Tomato black ring virus, Tomato black ring virus satellite, Tomato bunchy top viroid,
tomato bushy stunt virus, Tomato bushy stunt virus satellite, Tomato golden mosaic virus, Tomato leaf crumple virus,
Tomato leaf curl virus, Tomato leafroll virus, Tomato mosaic virus, Tomato mottle virus, Tomato pale chlorosis virus,
Tomato planta macho viroid, Tomato pseudo-curly top virus, Tomato ringspot virus, Tomato spotted wilt virus, Tomato
top necrosis virus, Tomato vein yellowing virus, Tomato yellow dwarf virus, Tomato yellow leaf curl virus, Tomato yellow
mosaic virus, Tomato yellow top virus, Tombusvirus, Tongan vanilla virus, Torovirus, Torque teno virus, Tortrix
loeflingiana NPV, Tortrix viridana NPV, Toscana virus, Tospovirus, Toxorhynchites brevipalpis NPV, Trabala vishnou
NPV, Tradescantia/Zebrina virus, Trager duck spleen necrosis virus, Tranosema sp. Virus, transforming virus, Tree
shrew adenovirus 1, Tree shrew herpesvims, Triatoma virus, Tribec virus, Trichiocampus irregularis NPV, Trichiocampus
viminalis NPV, Trichomonas vaginalis virus, Trichoplusia ni cypovirus 5, Trichoplusia ni granulovirus, Trichoplusia ni
MNPV, Trichoplusia ni Single SNPV, Trichoplusia ni virus,Trichosanthes mottle virus, Triticum aestivum chlorotic spot
virus, Trivittatus virus, Trombetas virus, Tropaeolum virus 1, Tropaeolum virus 2, Trubanarnan virus, Tsuruse virus,
Tucunduba virus, Tulare apple mosaic virus, Tulip band breaking virus, Tulip breaking virus, Tulip chlorotic blotch virus,
Tulip top breaking virus, Tulip virus X, tumor virus, Tupaia virus, Tupaiid herpesvirus 1, Turbot herpesvirus, Turbot
reovirus, Turkey adenoviruses 1 to 3, Turkey coronavirus, Turkey herpesvirus 1, turkey rhinotracheitis virus, turkeypox
virus, Turlock virus, Turnip crinkle virus, Turnip crinkle virus satellite, Turnip mild yellows virus, Turnip mosaic virus,
Turnip rosette virus, turnip yellow mosaic virus, Turuna virus, Tymovirus, Tyuleniy virus, type Cretroviruses, type D
oncovirus, type D retrovirus group, Uasin Gishu disease virus, Uganda S virus, Ugymyia sericariae NPV, ulcerative
disease rhabdovirus, Ullucus mild mottle virus, Ullucus mosaic virus, Ullucus virus C, Umatilla virus, Umbre virus, Una
virus, Upolu virus, UR2 sarcoma virus, Uranotaenia sapphirina NPV, Urbanus proteus NPV, Urucuri virus, Ustilago
maydis virus 1, Ustilago maydis virus 4, Ustilago maydis virus 6, Usutu virus, Utinga virus, Utive virus, Uukuniemi virus
group, Vaccinia virus, Vaeroy virus, Vallota mosaic virus, Vanessa atalanta NPV, Vanessa cardui NPV, Vanessa prorsa
NPV, Vanilla mosaic virus, Vanilla necrosis virus, Varicella zoster virus, Varicellovirus, Varicola virus, variola major virus,
variola virus, Vasin Gishu disease virus, Vellore virus, Velvet tobacco mottle virus, Velvet tobacco mottle virus satellite,
Venezuelan equine encephalitis virus, Venezuelan equine encephalomyelitis virus, Venezuelan hemorrhagic fever virus,
Vesicular stomatitis virus, Vesiculovirus, Vibrio phage 06N-22P, Vibrio phage 06N-58P, Vibrio phage 4996, Vibrio phage
a3a, Vibrio phage I, Vibrio phage II, Vibrio phage m, Vibrio phage IV, Vibrio phage kappa, Vibrio phage nt-1, Vibrio
phage OXN-52P, Vibrio phage OXN-IOOP, Vibrio phage v6, Vibrio phage Vfl2, Vibrio phage Vf33, Vibrio phage VP1,
Vibrio phage VP11, Vibrio phage VP3, Vibrio phage VP5, Vibrio phage X29, Vicia cryptic virus, Vigna sinensis mosaic
virus, Vilyuisk virus, Vinces virus, Viola mottle virus, viper retrovirus, viral haemorrhagic septicemia virus, virus-like particle,
Visna Maedi virus, Visna virus, Voandzeia mosaic virus, Voandzeia necrotic mosaic virus, volepox virus, Wad Medani
virus, Wallal virus, Walleye epidermal hyperplasia, Walrus calicivirus, Wanowrie virus, Warrego virus, Watermelon
chlorotic stunt virus, Watermelon curly mottle virus, Watermelon mosaic virus 1, Watermelon mosaic virus 2, Weddel
water-borne virus, Weldona virus, Wesselsbron virus, West Nile virus, western equine encephalitis virus, western equine
encephalomyelitis virus, Wexford virus, Whataroa virus, Wheat American striate mosaic virus, Wheat chlorotic streak
virus, Wheat dwarf virus, Wheat rosette stunt virus, Wheat streak mosaic virus, Wheat yellow leaf virus, Wheat yellow
mosaic virus, White bryony virus, White clover cryptic virus 1, White clover cryptic virus 2, White clover cryptic virus 3,
White clover mosaic virus, White lupinrnosaic virus, Wild cucumber mosaic virus, Wild potato mosaic virus, Wildbeest
herpesvirus, Wineberry latent virus, Winter wheat mosaic virus, Winter wheat Russian mosaic virus, Wiseana cervinata
virus, Wiseana signata virus, Wiseana umbraculata virus, Wissadula mosaic virus, Wisteria vein mosaic virus, Witwatersrand
virus, Wongal virus, Wongorr virus, Winter Vomiting Virus, woodchuck hepatitis B virus, Woodchuck herpesvirus marmota
1, woolly monkey sarcoma virus, wound tumor virus, WRSV virus, WVU virus 2937, WW virus 71 to 212, Wyeomyia smithii
NPV, Wyeomyia virus, Xanthomonas phage Cf, Xanthomonas phage Cflt, Xanthomonas phage RR66, Xanthomonas phage
Xf, Xanthomonas phage Xf2, Xanthomonas phage XP5, Xenopus virus T21, Xiburema virus, Xingu virus, Xylena curvimacula
NPV, Y73 sarcoma virus, Yaba monkey tumor virus, Yaba-1 virus, Yaba-7 virus, Yacaaba virus, Yam mosaic virus, Yaounde
virus, Yaquina Head virus, Yatapoxvirus, Yellow fever virus, Yogue virus, Yokapox virus, Yokase virus, Yponomeuta
cognatella NPV, Yponomeuta evonymella NPV, Yponomeuta malinellus NPV, Yponomeuta padella NPV, Yucca baciliform
virus, Yug Bogdanovac virus, Zaliv Terpeniya virus, Zea mays virus, Zegla virus, Zeiraphera diniana virus, Zeiraphera
pseudotsugana NPV, Zika virus, Zirqa virus, Zoysia mosaic virus, Zucchini yellow fleck virus, Zucchini yellow mosaic virus,
and Zygocactus virus.

### Archeology and Paleopathology

Evidence of infection in archeological findings and fossil remains is a subject of interest for paleopathologists and scientists who study e.g. occurrences of injuries and illness in extinct life forms. Accordingly, one embodiment of the invention relates to the use of the method disclosed herein within archeology.

Paleopathology is the study of ancient diseases. Accordingly, one embodiment of the invention relates to the use of the method disclosed herein within paleopathology. One embodiment thereof relates to the use of the method disclosed herein or the determination of e.g. sex and what sort of diseases the individual may have had, such as e.g. tuberculosis or syphilis

In one embodiment wherein the method is used in the field of archeology, and paleopathology, the test polynucleotide is made of a naturally occurring nucleotides.

### Food Contamination

Food contamination refers to the presence in food of anything which is not intended to be inside the food product in question, such as e.g. non-declared food components, harmful chemicals and microorganisms which can cause consumer illness. The method disclosed herein may be used for detection of such food contamination by identification of target polynucleotide not declared to be inside the specific food product in question. Accordingly, one embodiment of the invention relates to the use of the method disclosed herein for the detection of food contaminants, such as e.g. contaminants selected from the group consisting of microbiological contaminants, genetically modified food and food comprising non-declared components.

In one embodiment wherein the method disclosed herein is used for detection of food contamination, the target polynucleotide may be made of naturally occurring nucleotides or which may be made of nucleotides which are not known to occur naturally or it may be made of any mixture thereof.

One specific embodiment of the invention, relates to the use of the method disclosed herein for detection of microbiological contaminants in food products. In a particular embodiment thereof, said microbiological contamination is caused by pathogenic bacteria, viruses, exotoxins or parasites that contaminate the food product in question. In a further particular embodiment thereof, said microbiological contamination arises from improper handling, preparation, or food storage. In a specific embodiment thereof, said microbiological contamination arises from lack of good hygiene practices before, during, and after food preparation. In an embodiment thereof, the microbiological contamination is caused by bacterial foodborne pathogens selected from the group consisting of Campylobacter jejuni, Clostridium perfringens, Salmonella, Escherichia coli O157:H7, Bacillus cereus, Escherichia coli such as enteroinvasive (EIEC), enteropathogenic (EPEC), enterotoxigenic (ETEC) or enteroaggregative (EAEC or EAgEC), Listeria monocytogenes, Shigella, Staphylococcus aureus, Staphylococcal enteritis, Streptococcus, Vibrio cholerae, including O1 and non-O1, Vibrio parahaemolyticus, Vibrio vulnificus, Yersinia enterocolitica, Yersinia pseudotuberculosis, Brucella, Corynebacterium ulcerans, Coxiella burnetii, Plesiomonas shigelloides, Clostridium botulinum, Clostridium perfringens, Staphylococcus aureus and Bacillus cereus.

Another specific embodiment of the invention relates to the use of the method disclosed herein for detection of genetically modified material in a food product. Genetically modified food is food derived from genetically modified organisms. One specific embodiment relates to the identification of genetically modified foods which are transgenic plant products such as e.g. soybean, corn, canola, and cotton seed oil. Another specific embodiment relates to the identification of generically modified foods which are animal products. One further specific embodiment thereof, relates to the identification of generically modified foods for safety reasons. Another specific embodiment thereof, relates to the identification of generically modified foods for ecological concerns.

Yet another specific embodiment of the invention relates to the use of the method disclosed herein for detection of non-declared components, such as e.g. porks meet in what is declared to be beef, veal, turkey, chicken, sheep or lamp.

### Environmental Pollution

Water pollution is the contamination of water bodies. The specific contaminants leading to pollution in water include e.g. pathogens. Accordingly, one embodiment of the invention relates to detection of pathogens in water, such as e.g. in water supply to the household and water in lakes, rivers, oceans and groundwater. A specific embodiment thereof relates to the detection of pathogens in water supply to the household.

In an embodiment wherein the target polynucleotide is used for detection of environmental pollution, the target polynucleotide may be made of naturally occurring polynucleotides or of nucleotides which are not known to occur naturally or of any mixture thereof.

Infectious diseases such as cholera and typhoid can be contracted from drinking contaminated water. Our whole body system can have a lot of harm if polluted water is consumed regularly.

Accordingly, in a particular embodiment, the method disclosed herein is used for the detection of one or more patogens in a water sample, such as e.g. one or more patogens selected from the group consisting of Burkholderia pseudomallei, Coliform bacterium, Cryptosporidium parvum, Giardia lamblia, Salmonella, Novovirus and other viruses and Parasitic worms (helminths). One specific embodiment thereof relates to the use of the method according to the invention for detection of Burkholderia pseudomallei in a water sample. One further specific embodiment thereof relates to the use of the method according to the invention for detection of Coliform bacterium in a water sample. One further specific embodiment thereof relates to the use of the method according to the invention for detection of Cryptosporidium parvum in a water sample. One further specific embodiment thereof relates to the use of the method according to the invention for detection of Giardia lamblia in a water sample. One further specific embodiment thereof relates to the use of the method according to the invention for detection of Salmonella in a water sample. One further specific embodiment thereof relates to the use of the method according to the invention for detection of Novovirus and other viruses in a water sample. One further specific embodiment thereof relates to the use of the method according to the invention for detection of Parasitic worms in a water sample.

### Items

Each of the below items 1-92 and each possible combination thereof is a separate embodiment within the meaning of the present invention and may be made the subject of one or more dependent or independent claims:
1. A method for capture of nucleotide such as single stranded target DNA or RNA from a sample comprising the steps of
   i) removal of one or more of the types of bases A, T, U, C or G from said target polynucleotide such as DNA or RNA thereby generating one or more abasic sites and
   ii) capture of said target polynucleotide such as DNA or RNA with a complementary probe comprising one or more intercalator molecules which can be inserted into one or more of the one ore more abasic sites.
1. The method for capture of single stranded target DNA according to item 1 comprising the steps of
   (i) providing double stranded target DNA
   (ii) destabilisation of said double stranded target DNA by removal of one or more of the types of bases A, T, U, C or G from said double stranded target DNA thereby generating one or more abasic sites
   (iii) denaturing of said destabilized double stranded target DNA to single stranded target DNA and
   (iv) capture of said single stranded target DNA with a complementary DNA probe comprising one or more intercalator molecules which can be inserted into the one ore more abasic sites.
2. The method according to any of the previous items wherein the complementary probe can be selected from the group consisting of a DNA probe, a RNA probe, a LNA probe and a PNA probe.
3. The method according to any of the previous items, wherein the method further comprises one or more washing steps in order to remove of unbound DNA and/or RNA.
4. The method according to any of the previous items, wherein the method further comprises conversion of one or more types of bases in the double stranded target DNA and/or single stranded DNA and/or RNA to another chemical entity.
5. The method according to item 5, wherein the method further comprises destabilisation of said double stranded target DNA by removal of one or more of the these chemical entities from said double stranded target DNA.
6. The method according to any of the previous items, wherein the method further comprises conversion of one or more C's in the target DNA and/or RNA to one or more U's.
7. The method according to item 7, wherein the conversion of one or more C's in the target DNA and/or RNA to one or more U's is preformed by bisulphite treatment.
8. The method according to any of the previous items, wherein A is removed from said double stranded target DNA and/or single stranded DNA and/or RNA.
9. The method according to any of the previous items, wherein T is removed from said double stranded target DNA and/or single stranded DNA and/or RNA.
10. The method according to any of the previous items, wherein C is removed from said double stranded target DNA and/or single stranded DNA and/or RNA.
11. The method according to any of the previous items, wherein G is removed from said double stranded target DNA and/or single stranded DNA and/or RNA.
12. The method according to any of the previous items, wherein U is removed from said double stranded target DNA and/or single stranded DNA and/or RNA.
13. The method according to any of items 9 to 13, wherein the removal is performed by one or more enzymes and/or physical stress.
14. The method according to item 13, wherein the removal of U is performed by use of uracil dehydrogenase.
15. The method according to item 9, wherein the removal of A is performed by adjustment of the pH value.
16. The method according to any of the previous items, wherein 1, 2, or 3 types of the bases from the target DNA and/or RNA is removed.
17. The method according to any of the previous items, wherein the total number of bases that are removed from the target DNA and/or RNA can be selected from the group consisting of 1, 2, 3, 4, 5, 6, 7, 8, 9, 10, 11, 12, 13, 14, 15, 16, 17, 18, 19, 20 and more than 20 bases.
18. The method according to any of the previous items, wherein the complementary probe comprises one or more intercalator molecules.
19. The method according to item 19, wherein the total number of intercalator molecules can be selected from the group consisting of 1, 2, 3, 4, 5, 6, 7, 8, 9, 10, 11, 12, 13, 14, 15, 16, 17, 18, 19, 20 and more than 20 intercalator molecules.
20. The method according to any of the previous items, wherein an intercalator molecule has been inserted into from 10% to 100% of the abasic sites in the target DNA and/or RNA such as from 10% to 20%, for example from 20% to 30%, such as from 30% to 40%, for example from 40% to 50%, such as from 50% to 60%, for example from 60% to 70%, such as from 70% to 80%, for example from 80% to 90%, such as from 90% to 100%, or any combination thereof.
21. The method according to any of the previous items, wherein an intercalator molecule has been inserted into more than 10% of the abasic sites in the target DNA and/or RNA, such as more than 20%, for example more than 30%, such as more than 40%, for example more than 50%, such as more than 60%, for example more than 70%, such as more than 80%, for example more than 90%, such as more than 95%, for example 100%.
22. The method according to items 21 and 22, wherein the insertion of the intercalator molecules results in increased melting temperature of the polynucleotide duplex consisting of the target DNA and/or RNA and the complementary probe.
23. The method according to any of the previous items, wherein the ratio between the number of intercalator molecules and the total number of bases in the complementary probe is from 1:50 to 1:2 such as from 1:50 to 1:40, for example 1:40 to 1:30, such as from 1:30 to 1:20, for example 1:20 to 1:10, such as from 1:10 to 1:5, for example 1:5 to 1:2, or any combination of these intervals.
24. The method according to any of the previous items, wherein the one or more intercalator molecules can be selected from the group consisting of TINA, INA, ortho-TINA, para-TINA, and AMANY.
25. The method according to any of the previous items, wherein size of the intercalator molecule is between 20 and 400 Å, such as from 20-40 Å, for example from 40-60 Å, such as from 60-80 Å, for example from 80-100 Å, such as from 100-120 Å, for example from 120-140 Å, such as from 140-160 Å, for example from 160-180 Å, such as from 180-200 Å, for example from 200-220 Å, such as from 220-240 Å, for example from 240-260 Å, such as from 260-280 Å, for example from 280-300 Å, such as from 300-320 Å, for example from 320-340 Å, such as from 340-360 Å, for example from 360-380 Å, such as from 380-400 Å, or any combination of these intervals.
26. The method according to any of the previous items, wherein the complementary probe comprises more than one type of intercalator molecules such as 2, 3, 4, 5 or more than 5 different types of intercalator molecules.
27. The method according to any of the previous items, wherein the complementary probe is connected to a support.
28. The method according to item 28, wherein the support is selected from the group consisting of particulate matters, beads, magnetic beads, non-magnetic beads, polystyrene beads, magnetic polystyrene beads, sepharose beads, sephacryl beads, polystyrene beads, agarose beads, polysaccharide beads, and polycarbamate beads.
29. The method according to item 29, wherein the support is a solid support.
30. The method according to item 30, wherein the solid support can be selected from the group consisting of microtiter plate or other plate formats, reagent tubes, glass slides or other supports for use in array or microarray analysis, tubings or channels of micro fluidic chambers or devices and Biacore chips.
31. The method according to any of the previous items, wherein the method further comprises use of a complementary detection probe comprising one or more labels.
32. The method according to any of the previous items, wherein the complementary probe comprises one or more labels.
33. The method according to any of item 32-33, wherein the one or more labels can be selected from the group consisting of biotin, a fluorescent label, 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, dyes, Cy2, Cy3, and Cy5, PerCP, phycobiliproteins, R-phycoerythrin (RPE), allophycoerythrin (APC), Texas Red, Princeston Red, Green fluorescent protein (GFP) and analogues thereof, conjugates of R-phycoerythrin or allophycoerythrin, inorganic fluorescent labels based on semiconductor nanocrystals (like quantum dot and Qdot™ nanocrystals), time-resolved fluorescent labels based on lanthanides like Eu3+ and Sm3+, haptens, DNP, digoxiginin, enzymic labels, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase and glucose oxidase (GO), luminiscence labels, luminol, isoluminol, acridinium esters, 1,2-dioxetanes, pyridopyridazines, radioactivity labels, isotopes of iodide, isotopes of cobalt, isotopes of elenium, isotopes of tritium, and isotopes of phosphor.
34. The method according to item 34, wherein the biotin is detected by use of streptavidin-R-phycoerythrine.
35. The method according to item 34, wherein the method further comprises a washing step prior to and/or after addition of the detection probe.
36. The method according to any of items 34-36, wherein complementary detection probe comprises one or more intercalator molecules.
37. The method according to item 37, wherein the total number of intercalator molecules can be selected from the group consisting of 1, 2, 3, 4, 5, 6, 7, 8, 9, 10, 11, 12, 13, 14, 15, 16, 17, 18, 19, 20 and more than 20 different or identical intercalator molecules.
38. The method according to any of the previous items, wherein the target DNA and/or RNA is derived from a human being, an animal, bacteria, vira, fungus, prions, protozoa and/or plant.
39. The method according to any of the previous items, wherein the target DNA and/or RNA is isolate from a sample from a human or animal body.
40. The method according to any of the previous items, wherein the target DNA and/or RNA is isolate from humans, animals, birds, insects, plants, algae, fungi's, yeast, viruses, bacteria and phages, multi-cellular and mono-cellular organisms.
41. The method according to any of the previous items, wherein the target DNA and/or RNA is isolate from faeces, blood, semen, cerebrospinal fluid, sputum, vaginal fluid, urine, saliva, hair, other bodily fluids, tissue samples, whole organs, sweat, tears or other sub-structures of humans or animals.
42. The method according to any of the previous items, wherein the total number of different target DNA sequences that are captured can be selected from the group consisting of 1, 2-5, 5-10, 10-15, 15-20, 20-25, 25-30, 30-35, 35-40, 40-45, 45-50, 50-55, 55-60, 60-65, 65-70, 70-75, 75-80, 80-85, 85-90, 95-100, 100-150, 150-200, 200-300, 300-500, 500-1000 and more than 1000 different, or any combination of these intervals.
43. A method for diagnosing of one or more diseases comprising use of the method according to any of items 1 to 43.
44. The method according to item 44, wherein the diagnosis comprises detection of target DNA and/or RNA from the genome of individual that is tested.
45. The method according to any of items 44-45, wherein the diagnosis comprises detection of target DNA and/or RNA which is not derived from the genome of the individual that is tested.
46. The method according to any of items 44-46, wherein the diagnosis comprises detection of target DNA and/or RNA from bacteria, vira, fungus, prions, protozoa and/or plant.
47. The method according to any of items 44-47 wherein the disease to be diagnosed is one or more genetic, i.e. hereditary diseases such one or more diseases selected from the group consisting of CADASIL syndrome; Carboxylase Deficiency, Multiple, Late-Onset; Cerebelloretinal Angiomatosis, familial; Crohn's disease, fibrostenosing; Deficiency disease, Phenylalanine Hydroxylase; Fabry disease; Hereditary coproporphyria; Incontinentia pigmenti; Microcephaly; Polycystic kidney disease; Siderius X-linked mental retardation syndrome caused by mutations in the PHF8 gene and achondroplasia.
48. The method according to any of items 44-47 wherein the disease to be diagnosed is one or more diseases such as hereditary diseases, cancer and infectious disease, headaches and other diseases wherein the method of the invention may be useful
49. The method according to any of items 44-47 and 49, wherein the disease to be diagnosed is cancer.
50. The method according to item 50, wherein the cancer is characterized by one or more one or more mutations in one or more the genes or genes encoding the proteins in the group consisting of 101F6, ABR, ADPRTL3, ANP32C, ANP32D, APC2, APC, ARF, ARHGAP8, ARH1, AT1G14320, ATM, ATP8A2, AXUD1, BAP1, BECN1, BIN1, BRCA1, BRCA2, BTG1, BTG2, C1orf11, C5orf4, C5orf7, Cables, CACNA2D2, CAP-1, CARS, CAV1, CD81, CDC23, CDK2AP1, CDKN1A, CDKN1C, CDKN2A, CDKN2B, CDKN2X, Ciao1-pending, CLCA2, CREBL2, CTNNA1, CUL2, CW17R, DAB2, DAF-18, D-APC, DBC2, DCC, DDX26, DEC1, DLC1, DLEC1, DLEU1, DLEU2, DLG1, DLGH1, DLGH3, DMBT1, DNAJA3, DOC-1, DPC4, DPH2L, EGR1, FABP3, FAT, FGL1, FHIT, FLJ10506, FOXD1, FOXP1, FT, FUS1, FUS2, GAK, GAS1, GAS11, GLD-1, GLTSCR1, GLTSCR2, GRC5, GRLF1, HDAC3, HEMK, HIC1, HRG22, HSAL2, HTS1, HYAL1, HYAL2, IFGBP7, IGSF4, ING1, ING1L, ING4, I(2)tid, I(3)mbn, I(3)mbt, LAPSER1, LATS1, LATS2, LDOC1, LOH11CR2A, LRP1B, LUCA3, MAD, MAP2K4, MAPKAPK3, MCC, MDC, MEN1, ML-1, MLH1, MRVI1, MTAP, MXI1, NAP1L4, NBR2, NF1, NF2, NORE1, NPR2L, NtRb1, OVCA2, PDGFRL, PHEMX, pHyde, PIG8, PIK3CG, PINX1, PLAGL1, PRDM2, PTCH, PTEN, PTPNI3, PTPRG, RASSF1, RB1, RBBP7, RBX1, RBM6, RECK, RFP2, RIS1, RPL10, RPS29, RRM1, S100A2, SEMA3B, SF1, SFRP1, SLC22A1L, SLC26A3, SMARCA4, ST7, ST7L, ST13, ST14, STIM1, TCEB2, THW, TIMP3, TP53, TP63, TRIM8, TSC2, TSG101, TSSC1, TSSC3, TSSC4, VHL, VhIh, WFDC1, WIT-1, WT1, and WWOX.
51. The method according to item 50, wherein the cancer is characterized by one or more tumor antigens selected from the group consisting of alpha-actinin-4, ARTC1, BCR-ABL fusion protein (b3a2), B-RAF, CASP-5, CASP-8, beta-catenin, Cdc27, CDK4, CDKN2A, COA-1, dek-can fusion protein, EFTUD2, Elongation factor 2, ETV6-AML1 fusion protein, FLT3-ITD, FN1, GPNMB, LDLR-fucosyl transferase AS fusion protein, HLA-A2^{d}, HLA-A11^{d}, hsp70-2, KIAAO205, MART2, ME1, MUM-1^{f}, MUM-2, MUM-3, neo-PAP, Myosin class I, NFYC, OGT, OS-9, P53, pml-RARalpha fusion protein, PRDX5, PTPRK, K-ras, N-ras, RBAF600, SIRT2, SNRPDI, SYT-SSX1 or —SSX2 fusion protein, Triosephosphate Isomerase, BAGE-1, GAGE-1,2,8, GAGE-3,4,5,6,7, GnIV^{f}, HERV-K-MEL, KK-LC-1, KM-HN-1, LAGE-1, MAGE-A1, MAGE-A2, MAGE-A3, MAGE-A4, MAGE-A6, MAGE-A9, MAGE-A10, MAGE-A12, MAGE-C2, mucin^{k}, NA-88, NY-ESO-1/LAGE-2, SAGE, Sp17, SSX-2, SSX-4, TRAG-3, and TRP2-INT2^{g}.
52. The method according to item 50 wherein the cancer is characterized by one or more mutations in one or more genes or genes selected from the group consisting of RASSF2 and SFRP2.
53. The method according to item 50 wherein the cancer is characterized by one or more mutations in one or more genes or genes selected from the group consisting of TFPI2, NDRG4, GATA4 or GATA5.
54. The method according to item 44, wherein the diagnosis comprises diagnosis of a foetus disorder.
55. Use of the method according to any of items 1-43 with respect to personalized medicine.
56. A method for quantitation of target RNA comprising use of the method according to any of the items 1-43.
57. A method for detection of one or more target DNA and/or RNA comprising use of the method according to any of items 1 to 43.
58. The method according to item 58, wherein the sample is a feed, food or drinking water.
59. The method according to any of items 57 to 59, wherein the target DNA and/or RNA is derived from a human being, an animal, bacteria, vira, fungus, prions, protozoa and/or plant.
60. The method according to items 39 and 60, wherein the bacteria is selected from the group consisting of Acetobacter aurantius, Acinetobacter species: Acinetobacter baumannii, Acinetobacter calcoaceticus, Acinetobacter johnsonii, Acinetobacter junii, Acinetobacter lwoffii, Acinetobacter radioresistens, Acinetobacter septicus, Acinetobacter schindleri, Acinetobacter ursingii; Actinomyces species: Actinomyces bovis, Actinomyces bowdenii, Actinomyces canis, Actinomyces cardiffensis, Actinomyces catuli, Actinomyces coleocanis, Actinomyces dentalis, Actinomyces denticolens, Actinomyces europaeus, Actinomyces funkei, Actinomyces georgiae, Actinomyces gerencseriae, Actinomyces graevenitzii, Actinomyces hongkongensis, Actinomyces hordeovulneris, Actinomyces howellii, Actinomyces humiferus, Actinomyces hyovaginalis, Actinomyces israelii, Actinomyces marimammalium, Actinomyces meyeri, Actinomyces naeslundii, Actinomyces nasicola, Actinomyces neuii, Actinomyces odontolyticus, Actinomyces oricola, Actinomyces radicidentis, Actinomyces radingae, Actinomyces slackii, Actinomyces streptomycini, Actinomyces suimastitidis, Actinomyces suis, Actinomyces turicensis, Actinomyces urogenitalis, Actinomyces vaccimaxillae, Actinomyces viscosus; Actinobacillus species: Actinobacillus actinomycetemcomitans, Actinobacillus arthritidis, Actinobacillus capsulatus, Actinobacillus delphinicola, Actinobacillus equuli, Actinobacillus hominis, Actinobacillus indolicus, Actinobacillus lignieresii, Actinobacillus minor, Actinobacillus muris, Actinobacillus pleuropneumoniae, Actinobacillus porcinus, Actinobacillus rossii, Actinobacillus scotiae, Actinobacillus seminis, Actinobacillus succinogenes, Actinobacillus suis, Actinobacillus ureae; Aeromonas species: Aeromonas allosaccharophila, Aeromonas bestiarum, Aeromonas bivalvium, Aeromonas encheleia, Aeromonas enteropelogenes, Aeromonas euchrenophila, Aeromonas hydrophila, Aeromonas ichthiosmia, Aeromonas jandaei, Aeromonas media, Aeromonas molluscorum, Aeromonas popoffli, Aeromonas punctata, Aeromonas salmonicida, Aeromonas schubertii, Aeromonas sharmana, Aeromonas simiae, Aeromonas sobria, Aeromonas veronii; Afipia felis, Agrobacterium species: Agrobacterium radiobacter, Agrobacterium rhizogenes, Agrobacterium rubi, Agrobacterium tumefaciens; Agromonas species, Alcaligenes species: Alcaligenes aquatilis, Alcaligenes eutrophus, Alcaligenes faecalis, Alcaligenes latus, Alcaligenes xylosoxidans; Alishewanella species, Alterococcus species, Anaplasma phagocytophilum, Anaplasma marginale, Aquamonas species, Arcanobacterium haemolyticum, Aranicola species, Arsenophonus species, Azotivirga species, Azotobacter vinelandii, Azotobacter chroococcum, Bacillary dysentery (Shigellosis), Bacillus species: Bacillus abortus (Brucella melitensis biovar abortus), Bacillus anthracis (Anthrax), Bacillus brevis, Bacillus cereus, Bacillus coagulans, Bacillus fusiformis, Bacillus globigii, Bacillus licheniformis, Bacillus megaterium, Bacillus mycoides, Bacillus natto, Bacillus stearothermophilus, Bacillus subtilis, Bacillus sphaericus, Bacillus thuringiensis; Bacteroides species: Bacteroides forsythus (Tannerella forsythensis), Bacteroides acidifaciens, Bacteroides distasonis (reclassified as Parabacteroides distasonis), Bacteroides gingivalis, Bacteroides gracilis, Bacteroides fragilis, Bacteroides oris, Bacteroides ovatus, Bacteroides putredinis, Bacteroides pyogenes, Bacteroides stercoris, Bacteroides suis, Bacteroides tectus, Bacteroides thetaiotaomicron, Bacteroides vulgatus; Bartonella species: Bartonella alsatica, Bartonella bacilliformis, Bartonella birtlesii, Bartonella bovis, Bartonella capreoli, Bartonella clarridgeiae, Bartonella doshiae, Bartonella elizabethae, Bartonella grahamii, Bartonella henselae (cat scratch fever), Bartonella koehlerae, Bartonella muris, Bartonella peromysci, Bartonella quintana, Bartonella rochalimae, Bartonella schoenbuchii, Bartonella talpae, Bartonella taylorii, Bartonella tribocorum, Bartonella vinsonii spp. Arupensis, Bartonella vinsonii spp. Berkhoffii, Bartonella vinsonii spp. Vinsonii, Bartonella washoensis; BCG (Bacille Calmette-Guerin), Bergeyella zoohelcum (Weeksella zoohelcum), Bifidobacterium bifidum, Blastobacter species, Blochmannia species, Bordetella species: ‘Bordetella ansorpii’, Bordetella avium, Bordetella bronchiseptica, Bordetella hinzii, Bordetella holmesii, Bordetella parapertussis, Bordetella pertussis (Whooping cough), Bordetella petrii, Bordetella trematum; Borrelia species, Borrelia burgdorferi, Borrelia afzelii, Borrelia anserina, Borrelia garinii, Borrelia valaisiana, Borrelia hermsii, Borrelia Parkeri, Borrelia recurrentis; Bosea species, Bradyrhizobium species, Brenneria species, Brucella species: Brucella abortus, Brucella canis, Brucella melitensis, Brucella neotomae, Brucella ovis, Brucella suis, Brucella pinnipediae; Buchnera species, Budvicia species, Burkholderia species: Burkholderia cepacia (Pseudomonas cepacia), Burkholderia mallei (Pseudomonas mallei/Actinobacillus mallei), Burkholderia pseudomallei (Pseudomonas pseudomallei); Buttiauxella species, Calymmatobacterium granulomatis, Campylobacter species: Campylobacter coli, Campylobacter concisus, Campylobacter curvus, Campylobacter fetus, Campylobacter gracilis, Campylobacter helveticus, Campylobacter hominis, Campylobacter hyointestinalis, Campylobacter insulaenigrae, Campylobacter jejuni, Campylobacter lanienae, Campylobacter lari, Campylobacter mucosalis, Campylobacter rectus, Campylobacter showae, Campylobacter sputorum, Campylobacter upsaliensis; Capnocytophaga canimorsus (Dysgonic fermenter type 2), Corynebacterium species, Cardiobacterium hominis, Cedecea species, Chlamydia species: Chlamydia trachomatis (Lymphogranuloma venereum), Chlamydia muridarum, Chlamydia suis; Chlamydophila species: Chlamydophila pneumoniae, Chlamydophila psittaci (Psittacosis), Chlamydophila pecorum, Chlamydophila abortus, Chlamydophila felis, Chlamydophila caviae; Citrobacter species: Citrobacter amalonaticus, Citrobacter braakii, Citrobacter farmeri, Citrobacter freundii, Citrobacter gillenii, Citrobacter intermedius, Citrobacter koseri aka Citrobacter diversus, Citrobacter murliniae, Citrobacter rodentium, Citrobacter sedlakii, Citrobacter werkmanii, Citrobacter youngae; Clostridium species: Clostridium botulinum, Clostridium difficile, Clostridium novyi, Clostridium septicum, Clostridium tetani (Tetanus), Clostridium welchii (Clostridium perfringens); Corynebacterium species: Corynebacterium diphtheriae (Diphtheria), Corynebacterium amycolatum, Corynebacterium aquaticum, Corynebacterium bovis, Corynebacterium equi, Corynebacterium flavescens, Corynebacterium glutamicum, Corynebacterium haemolyticum, Corynebacterium jeikeiun (corynebacteria of group JK), Corynebacterium minutissimum (Erythrasma), Corynebacterium parvum (also called Propionibacterium acnes), Corynebacterium pseudodiptheriticum (also called Corynebacterium hofmannii), Corynebacterium pseudotuberculosis (also called Corynebacterium ovis), Corynebacterium pyogenes, Corynebacterium urealyticum (corynebacteria of group D2), Corynebacterium renale, Corynebacterium striatum, Corynebacterium tenuis (Trichomycosis palmellina, Trichomycosis axillaris), Corynebacterium ulcerans, Corynebacterium xerosis; Coxiella burnetii (Q fever), Cronobacter species: Cronobacter sakazakii, Cronobacter malonaticus, Cronobacter turicensis, Cronobacter muytjensii, Cronobacter dublinensis; Delftia acidovorans (Comamonas acidovorans), Dickeya species, Edwardsiella species, Eikenella corrodens, Enterobacter species: Enterobacter aerogenes, Enterobacter cloacae, Enterobacter sakazakii; Enterococcus species: Enterococcus avium, Enterococcus durans, Enterococcus faecalis (Streptococcus faecalis/Streptococcus Group D), Enterococcus faecium, Enterococcus solitarius, Enterococcus galllinarum, Enterococcus maloratus; Ehrlichia chaffeensis, Erysipelothrix rhusiopathiae, Erwinia species, Escherichia species: Escherichia adecarboxylata, Escherichia albertii, Escherichia blattae, Escherichia coli, Escherichia fergusonii, Escherichia hermannii, Escherichia vulneris; Ewingella species, Flavobacterium species: Flavobacterium aquatile, Flavobacterium branchiophilum, Flavobacterium columnare, Flavobacterium flevense, Flavobacterium gondwanense, Flavobacterium hydatis, Flavobacterium johnsoniae, Flavobacterium pectinovorum, Flavobacterium psychrophilum, Flavobacterium saccharophilum, Flavobacterium salegens, Flavobacterium scophthalmum, Flavobacterium succinans; Francisella tularensis (Tularaemia), Francisella novicida, Francisella philomiragia, Fusobacterium species: Fusobacterium necrophorum (Lemierre syndrome/Sphaerophorus necrophorus), Fusobacterium nucleatum, Fusobacterium polymorphum, Fusobacterium novum, Fusobacterium mortiferum, Fusobacterium varium; Gardnerella vaginalis, Gemella haemolysans, Gemella morbillorum (Streptococcus morbillorum), Grimontella species, Haemophilus species: Haemophilus aegyptius (Koch-Weeks bacillus), Haemophilus aphrophilus, Haemophilus avium, Haemophilus ducreyi (Chancroid), Haemophilus felis, Haemophilus haemolyticus, Haemophilus influenzae (Pfeiffer bacillus), Haemophilus paracuniculus, Haemophilus parahaemolyticus, Haemophilus parainfluenzae, Haemophilus paraphrophilus (Aggregatibacter aphrophilus), Haemophilus pertussis, Haemophilus pittmaniae, Haemophilus somnus, Haemophilus vaginalis; Hafnia species, Hafnia alvei, Helicobacter species: Helicobacter acinonychis, Helicobacter anseris, Helicobacter aurati, Helicobacter bilis, Helicobacter bizzozeronii, Helicobacter brantae, Helicobacter canadensis, Helicobacter canis, Helicobacter cholecystus, Helicobacter cinaedi, Helicobacter cynogastricus, Helicobacter felis, Helicobacter fennelliae, Helicobacter ganmani, Helicobacter heilmannii (Gastrospirillum hominis), Helicobacter hepaticus, Helicobacter mesocricetorum, Helicobacter marmotae, Helicobacter muridarum, Helicobacter mustelae, Helicobacter pametensis, Helicobacter pullorum, Helicobacter pylori (stomach ulcer), Helicobacter rappini, Helicobacter rodentium, Helicobacter salomonis, Helicobacter trogontum, Helicobacter typhlonius, Helicobacter winghamensis; Human granulocytic ehrlichiosis (Anaplasma phagocytophilum/Ehrlichia phagocytophila), Human monocytotropic ehrlichiosis (Monocytic ehrlichiosis/Ehrlichia chaffeensis), Klebsiella species: Klebsiella granulomatis (Calymmatobacterium granulomatis), Klebsiella mobilis, Klebsiella ornithinolytica, Klebsiella oxytoca, Klebsiella ozaenae, Klebsiella planticola, Klebsiella pneumoniae, Klebsiella rhinoscleromatis, Klebsiella singaporensis, Klebsiella terrigena, Klebsiella trevisanii, Klebsiella variicola; Kingella kingae, Kluyvera species, Lactobacillus species: Lactobacillus acetotolerans, Lactobacillus acidifarinae, Lactobacillus acidipiscis, Lactobacillus acidophilus (Doderlein bacillus), Lactobacillus agilis, Lactobacillus algidus, Lactobacillus alimentarius, Lactobacillus amylolyticus, Lactobacillus amylophilus, Lactobacillus amylotrophicus, Lactobacillus amylovorus, Lactobacillus animalis, Lactobacillus antri, Lactobacillus apodemi, Lactobacillus aviarius, Lactobacillus bifermentans, Lactobacillus brevis, Lactobacillus buchneri, Lactobacillus camelliae, Lactobacillus casei, Lactobacillus catenaformis, Lactobacillus ceti, Lactobacillus coleohominis, Lactobacillus collinoides, Lactobacillus composti, Lactobacillus concavus, Lactobacillus coryniformis, Lactobacillus crispatus, Lactobacillus crustorum, Lactobacillus curvatus, Lactobacillus delbrueckii, Lactobacillus delbrueckii subsp. Bulgaricus, Lactobacillus delbrueckii subsp. Lactis, Lactobacillus diolivorans, Lactobacillus equi, Lactobacillus equigenerosi, Lactobacillus farraginis, Lactobacillus farciminis, Lactobacillus fermentum, Lactobacillus formicalis, Lactobacillus fructivorans, Lactobacillus frumenti, Lactobacillus fuchuensis, Lactobacillus gallinarum, Lactobacillus gasseri, Lactobacillus gastricus, Lactobacillus ghanensis, Lactobacillus graminis, Lactobacillus hammesii, Lactobacillus hamsteri, Lactobacillus harbinensis, Lactobacillus hayakitensis, Lactobacillus helveticus, Lactobacillus hilgardii, Lactobacillus homohiochii, Lactobacillus iners, Lactobacillus ingluviei, Lactobacillus intestinalis, Lactobacillus jensenii, Lactobacillus johnsonii, Lactobacillus kalixensis, Lactobacillus kefiranofaciens, Lactobacillus kefiri, Lactobacillus kimchii, Lactobacillus kitasatonis, Lactobacillus kunkeei, Lactobacillus leichmannii, Lactobacillus lindneri, Lactobacillus malefermentans, Lactobacillus mall, Lactobacillus manihotivorans, Lactobacillus mindensis, Lactobacillus mucosae, Lactobacillus murinus, Lactobacillus nagelii, Lactobacillus namurensis, Lactobacillus nantensis, Lactobacillus oligofermentans, Lactobacillus oris, Lactobacillus panis, Lactobacillus pantheris, Lactobacillus parabrevis, Lactobacillus parabuchneri, Lactobacillus paracollinoides, Lactobacillus parafarraginis, Lactobacillus parakefiri, Lactobacillus paralimentarius, Lactobacillus paraplantarum, Lactobacillus pentosus, Lactobacillus perolens, Lactobacillus plantarum, Lactobacillus pontis, Lactobacillus psittaci, Lactobacillus rennini, Lactobacillus reuteri, Lactobacillus rhamnosus, Lactobacillus rimae, Lactobacillus rogosae, Lactobacillus rossiae, Lactobacillus ruminis, Lactobacillus saerimneri, Lactobacillus sakei, Lactobacillus salivarius, Lactobacillus sanfranciscensis, Lactobacillus satsumensis, Lactobacillus secaliphilus, Lactobacillus sharpeae, Lactobacillus siliginis, Lactobacillus spicheri, Lactobacillus suebicus, Lactobacillus thailandensis, Lactobacillus ultunensis, Lactobacillus vaccinostercus, Lactobacillus vaginalis, Lactobacillus versmoldensis, Lactobacillus vini, Lactobacillus vitulinus, Lactobacillus zeae, Lactobacillus zymae; Leclercia species, Legionella species: Legionella adelaidensis, Legionella anisa, Legionella beliardensis, Legionella birminghamensis, Legionella bozemanii, Legionella brunensis, Legionella busanensis, Legionella cherrii, Legionella cincinnatiensis, Legionella donaldsonii, Legionella drancourtii, Legionella drozanskii, Legionella erythra, Legionella fairfieldensis, Legionella fallonii, Legionella feeleii, Legionella geestiana, Legionella genomospecies, Legionella gratiana, Legionella gresilensis, Legionella hackeliae, Legionella impletisoli, Legionella israelensis, Legionella jamestowniensis, ‘Candidatus Legionella jeonii’, Legionella jordanis, Legionella lansingensis, Legionella londiniensis, Legionella longbeachae, Legionella lytica, Legionella maceachernii, Legionella micdadei, Legionella moravica, Legionella nautarum, Legionella oakridgensis, Legionella parisiensis, Legionella pneumophila, Legionella quateirensis, Legionella quinlivanii, Legionella rowbothamii, Legionella rubrilucens, Legionella sainthelensi, Legionella santicrucis, Legionella shakespearei, Legionella spiritensis, Legionella steigerwaltii, Legionella taurinensis, Legionella tucsonensis, Legionella wadsworthii, Legionella waltersii, Legionella worsleiensis, Legionella yabuuchiae; Leminorella species, Leptospira species: Leptospira interrogans, Leptospira kirschneri, Leptospira noguchii, Leptospira alexanderi, Leptospira weilii, Leptospira genomospecies 1, Leptospira borgpetersenii, Leptospira santarosai, Leptospira inadai, Leptospira fainei, Leptospira broomii, Leptospira licerasiae, Leptospira biflexa, Leptospira meyeri, Leptospira wolbachii, Leptospira genomospecies 3, Leptospira genomospecies 4, Leptospira genomospecies 5; Lepromatous leprosy (Danielssen-Boeck disease), Leptospira canicola, Leptospira hebdomadis, Leptospirosis (Weil disease/Leptospira icterohaemorrhagiae/Leptospira interrogans serovar icterohaemorrhagiae), Leptotrichia, Leuconostoc species: Leuconostoc carnosum, Leuconostoc citreum, Leuconostoc durionis, Leuconostoc fallax, Leuconostoc ficulneum, Leuconostoc fructosum, Leuconostoc garlicum, Leuconostoc gasicomitatum, Leuconostoc gelidum, Leuconostoc inhae, Leuconostoc kimchii, Leuconostoc lactis, Leuconostoc mesenteroides, Leuconostoc pseudoficulneum, Leuconostoc pseudomesenteroides; Listeria species: Listeria grayi, Listeria innocua, Listeria ivanovii, Listeria monocytogenes (Listeriosis), Listeria seeligeri, Listeria welshimeri; Methanobacterium extroquens, Microbacterium multiforme, Micrococcus species: Micrococcus antarcticus, Micrococcus flavus, Micrococcus luteus, Micrococcus lylae, Micrococcus mucilaginosis, Micrococcus roseus, Micrococcus sedentarius; Mobiluncus, Moellerella species, Morganella species, Moraxella species: Moraxella atlantae, Moraxella boevrei, Moraxella bovis, Moraxella canis, Moraxella caprae, Moraxella catarrhalis (Branhamella catarrhalis), Moraxella caviae, Moraxella cuniculi, Moraxella equi, Moraxella lacunata, Moraxella lincolnii, Moraxella nonliquefaciens, Moraxella oblonga, Moraxella osloensis, Moraxella saccharolytica; Morganella morganii, Mycobacterium species: Mycobacterium abscessus, Mycobacterium africanum, Mycobacterium agri, Mycobacterium aichiense, Mycobacterium alvei, Mycobacterium arupense, Mycobacterium asiaticum, Mycobacterium aubagnense, Mycobacterium aurum, Mycobacterium austroafricanum, Mycobacterium avium (Battey disease/Lady Windermere syndrome), Mycobacterium avium paratuberculosis (implicated in Crohn's disease in humans and Johne's disease in sheep), Mycobacterium avium silvaticum, Mycobacterium avium “hominissuis”, Mycobacterium colombiense, Mycobacterium boenickei, Mycobacterium bohemicum, Mycobacterium bolletii, Mycobacterium botniense, Mycobacterium bovis (Bovine tuberculosis), Mycobacterium branderi, Mycobacterium brisbanense, Mycobacterium brumae, Mycobacterium canariasense, Mycobacterium caprae, Mycobacterium celatum, Mycobacterium chelonae, Mycobacterium chimaera, Mycobacterium chitae, Mycobacterium chlorophenolicum, Mycobacterium chubuense, Mycobacterium conceptionense, Mycobacterium confluentis, Mycobacterium conspicuum, Mycobacterium cookii, Mycobacterium cosmeticum, Mycobacterium diernhoferi, Mycobacterium doricum, Mycobacterium duvalii, Mycobacterium elephantis, Mycobacterium fallax, Mycobacterium farcinogenes, Mycobacterium flavescens, Mycobacterium florentinum, Mycobacterium fluoroanthenivorans, Mycobacterium fortuitum, Mycobacterium fortuitum subsp. Acetamidolyticum, Mycobacterium frederiksbergense, Mycobacterium gadium, Mycobacterium gastri, Mycobacterium genavense, Mycobacterium gilvum, Mycobacterium goodii, Mycobacterium gordonae (Mycobacterium aquae), Mycobacterium haemophilum, Mycobacterium hassiacum, Mycobacterium heckeshornense, Mycobacterium heidelbergense, Mycobacterium hiberniae, Mycobacterium hodleri, Mycobacterium holsaticum, Mycobacterium houstonense, Mycobacterium immunogenum, Mycobacterium interjectum, Mycobacterium intermedium, Mycobacterium intracellulare, Mycobacterium kansasii, Mycobacterium komossense, Mycobacterium kubicae, Mycobacterium kumamotonense, Mycobacterium lacus, Mycobacterium lentiflavum, Mycobacterium leprae (causes leprosy or Hansen disease/Hanseniasis), Mycobacterium lepraemurium, Mycobacterium madagascariense, Mycobacterium mageritense, Mycobacterium malmoense, Mycobacterium marinum (Fish tank granuloma), Mycobacterium massiliense, Mycobacterium microti, Mycobacterium monacense, Mycobacterium montefiorense, Mycobacterium moriokaense, Mycobacterium mucogenicum, Mycobacterium murale, Mycobacterium nebraskense, Mycobacterium neoaurum, Mycobacterium neworleansense, Mycobacterium nonchromogenicum, Mycobacterium novocastrense, Mycobacterium obuense, Mycobacterium palustre, Mycobacterium parafortuitum, Mycobacterium parascrofulaceum, Mycobacterium parmense, Mycobacterium peregrinum, Mycobacterium phlei, Mycobacterium phocaicum, Mycobacterium pinnipedii, Mycobacterium porcinum, Mycobacterium poriferae, Mycobacterium pseudoshottsii, Mycobacterium pulveris, Mycobacterium psychrotolerans, Mycobacterium pyrenivorans, Mycobacterium rhodesiae, Mycobacterium saskatchewanense, Mycobacterium scrofulaceum, Mycobacterium senegalense, Mycobacterium seoulense, Mycobacterium septicum, Mycobacterium shimoidei, Mycobacterium shottsii, Mycobacterium simiae, Mycobacterium smegmatis, Mycobacterium sphagni, Mycobacterium szulgai, Mycobacterium terrae, Mycobacterium thermoresistibile, Mycobacterium tokaiense, Mycobacterium triplex, Mycobacterium triviale, Mycobacterium tuberculosis (major cause of human tuberculosis), Mycobacterium bovis, Mycobacterium africanum, Mycobacterium canetti, Mycobacterium caprae, Mycobacterium pinnipedii′, Mycobacterium tusciae, Mycobacterium ulcerans (causes Bairnsdale ulcer/Buruli ulcer), Mycobacterium vaccae, Mycobacterium vanbaalenii, Mycobacterium wolinskyi, Mycobacterium xenopi; Mycoplasma species: Mycoplasma fermentans, Mycoplasma genitalium, Mycoplasma hominis, Mycoplasma penetrans, Mycoplasma phocacerebrale, Mycoplasma pneumoniae, Nanukayami (Seven-day fever/Gikiyami), Neisseria species: Neisseria gonorrhoea (Gonococcus/Gonorrhea), Neisseria meningiditis (Meningococcus), Neisseria sicca, Neisseria cinerea, Neisseria elongata, Neisseria flavescens, Neisseria lactamica, Neisseria mucosa, Neisseria polysaccharea, Neisseria subflava; Nitrobacter species, Nocardia species: Nocardia asteroides, Nocardia brasiliensis, Nocardia caviae; Noma (cancrum oris/gangrenous stomatitis), Obesumbacterium, Oligotropha species, Orientia tsutsugamushi (Scrub typhus), Oxalobacter formigenes, Pantoea species: Pantoea agglomerans, Pantoea ananatis, Pantoea citrea, Pantoea dispersa, Pantoea punctata, Pantoea stewartii, Pantoea terrea; Pasteurella species: Pasteurella aerogenes, Pasteurella anatis, Pasteurella avium, Pasteurella bettyae, Pasteurella caballi, Pasteurella canis, Pasteurella dagmatis, Pasteurella gallicida, Pasteurella gallinarum, Pasteurella granulomatis, Pasteurella langaaensis, Pasteurella lymphangitidis, Pasteurella mairii, Pasteurella multocida, Pasteurella pneumotropica, Pasteurella skyensis, Pasteurella stomatis, Pasteurella testudinis, Pasteurella trehalosi, Pasteurella tularensis, Pasteurella ureae, Pasteurella volantium; Pediococcus species: Pediococcus acidilactici, Pediococcus cellicola, Pediococcus claussenii, Pediococcus damnosus, Pediococcus dextrinicus, Pediococcus ethanolidurans, Pediococcus inopinatus, Pediococcus parvulus, Pediococcus pentosaceus, Pediococcus stilesii; Peptostreptococcus species: Peptostreptococcus anaerobius, Peptostreptococcus asaccharolyticus, Peptostreptococcus harei, Peptostreptococcus hydrogenalis, Peptostreptococcus indoliticus, Peptostreptococcus ivorii, Peptostreptococcus lacrimalis, Peptostreptococcus lactolyticus, Peptostreptococcus magnus, Peptostreptococcus micros, Peptostreptococcus octavius, Peptostreptococcus prevotii, Peptostreptococcus tetradius, Peptostreptococcus vaginalis; Photorhabdus species, Photorhizobium species, Plesiomonas shigelloides, Porphyromonas gingivalis, Pragia species, Prevotella, Propionibacterium species: Propionibacterium acnes, Propionibacterium propionicus; Proteus species: Proteus mirabilis, Proteus morganii, Proteus penneri, Proteus rettgeri, Proteus vulgaris; Providencia species: Providencia friedericiana, Providencia stuartii; Pseudomonas species: Pseudomonas aeruginosa, Pseudomonas alcaligenes, Pseudomonas anguilliseptica, Pseudomonas argentinensis, Pseudomonas borbori, Pseudomonas citronellolis, Pseudomonas flavescens, Pseudomonas mendocina, Pseudomonas nitroreducens, Pseudomonas oleovorans, Pseudomonas pseudoalcaligenes, Pseudomonas resinovorans, Pseudomonas straminea, Pseudomonas aurantiaca, Pseudomonas aureofaciens, Pseudomonas chlororaphis, Pseudomonas fragi, Pseudomonas lundensis, Pseudomonas taetrolens, Pseudomonas antarctica, Pseudomonas azotoformans, Pseudomonas brassicacearum, Pseudomonas brenneri, Pseudomonas cedrina, Pseudomonas corrugate, Pseudomonas fluorescens, Pseudomonas gessardii, Pseudomonas libanensis, Pseudomonas mandelii, Pseudomonas marginalis, Pseudomonas mediterranea, Pseudomonas meridiana, Pseudomonas migulae, Pseudomonas mucidolens, Pseudomonas orientalis, Pseudomonas panacis, Pseudomonas proteolytica, Pseudomonas rhodesiae, Pseudomonas synxantha, Pseudomonas thivervalensis, Pseudomonas tolaasii, Pseudomonas veronii, Pseudomonas denitrificans, Pseudomonas pertucinogena, Pseudomonas cremoricolorata, Pseudomonas fulva, Pseudomonas monteilii, Pseudomonas mosselii, Pseudomonas oryzihabitans, Pseudomonas parafulva, Pseudomonas plecoglossicida, Pseudomonas putida, Pseudomonas balearica, Pseudomonas luteola, Pseudomonas stutzeri, Pseudomonas amygdale, Pseudomonas avellanae, Pseudomonas caricapapayae, Pseudomonas cichorii, Pseudomonas coronafaciens, Pseudomonas ficuserectae, Pseudomonas meliae, Pseudomonas savastanoi, Pseudomonas syringae, Pseudomonas viridiflava, Pseudomonas abietaniphila, Pseudomonas acidophila, Pseudomonas agarici, Pseudomonas alcaliphila, Pseudomonas alkanolytica, Pseudomonas amyloderamosa, Pseudomonas asplenii, Pseudomonas azotifigens, Pseudomonas cannabina, Pseudomonas coenobios, Pseudomonas congelans, Pseudomonas costantinii, Pseudomonas cruciviae, Pseudomonas deihiensis, Pseudomonas excibis, Pseudomonas extremorientalis, Pseudomonas frederiksbergensis, Pseudomonas fuscovaginae, Pseudomonas gelidicola, Pseudomonas grimontii, Pseudomonas indica, Pseudomonas jessenii, Pseudomonas jinjuensis, Pseudomonas kilonensis, Pseudomonas knackmussii, Pseudomonas koreensis, Pseudomonas lini, Pseudomonas lutea, Pseudomonas moraviensis, Pseudomonas otitidis, Pseudomonas pachastrellae, Pseudomonas palleroniana, Pseudomonas papaveris, Pseudomonas peli, Pseudomonas perolens, Pseudomonas poae, Pseudomonas pohangensis, Pseudomonas psychrophila, Pseudomonas psychrotolerans, Pseudomonas rathonis, Pseudomonas reptilivora, Pseudomonas resiniphila, Pseudomonas rhizosphaerae, Pseudomonas rubescens, Pseudomonas salomonii, Pseudomonas segitis, Pseudomonas septica, Pseudomonas simiae, Pseudomonas suis, Pseudomonas thermotolerans, Pseudomonas tremae, Pseudomonas trivialis, Pseudomonas turbinellae, Pseudomonas tuticorinensis, Pseudomonas umsongensis, Pseudomonas vancouverensis, Pseudomonas vranovensis, Pseudomonas xanthomarina; Rahnella species, Ralstonia species: Ralstonia basilensis, Ralstonia campinensis, Ralstonia eutropha, Ralstonia gilardii, Ralstonia insidiosa, Ralstonia mannitolilytica, Ralstonia metallidurans, Ralstonia paucula, Ralstonia pickettii, Ralstonia respiraculi, Ralstonia solanacearum, Ralstonia syzygii, Ralstonia taiwanensis; Raoultella species, Rhodoblastus species, Rhodopseudomonas species, Rhinoscleroma, Rhizobium radiobacter, Rhodococcus equi, Rickettsia species: Rickettsia africae, Rickettsia akari, Rickettsia australis, Rickettsia conorii, Rickettsia felis, Rickettsia japonica, Rickettsia mooseri, Rickettsia prowazekii (Typhus fever), Rickettsia rickettsii, Rickettsia siberica, Rickettsia typhi, Rickettsia conorii, Rickettsia africae, Rickettsia psittaci, Rickettsia quintana, Rickettsia rickettsii, Rickettsia trachomae; Rothia dentocariosa, Salmonella species: Salmonella arizonae, Salmonella bongori, Salmonella enterica, Salmonella enteriditis, Salmonella paratyphi, Salmonella typhi (Typhoid fever), Salmonella typhimurium, Salmonella salamae, Salmonella arizonae, Salmonella diarizonae, Salmonella houtenae, Salmonella indica; Samsonia species, Serratia species: Serratia entomophila, Serratia ficaria, Serratia fonticola, Serratia grimesii, Serratia liquefaciens, Serratia marcescens, Serratia odoriferae, Serratia plymuthica, Serratia proteamaculans, Serratia quinivorans, Serratia rubidaea, Serratia ureilytica; Shewanella putrefaciens, Shigella boydii, Shigella dysenteriae, Shigella flexneri, Shigella sonnei, Sodalis species, Spirillum species: Spirillum minus rat bite fever, Staphylococcus species: Staphylococcus aureus, Staphylococcus auricularis, Staphylococcus capitis, Staphylococcus caprae, Staphylococcus cohnii, Staphylococcus epidermidis, Staphylococcus felis, Staphylococcus haemolyticus, Staphylococcus hominis, Staphylococcus intermedius, Staphylococcus lugdunensis, Staphylococcus pettenkoferi, Staphylococcus saprophyticus, Staphylococcus schleiferi, Staphylococcus simulans, Staphylococcus vitulus, Staphylococcus warneri, Staphylococcus xylosus; Stenotrophomonas species: Stenotrophomonas acidaminiphila, Stenotrophomonas dokdonensis, Stenotrophomonas koreensis, Stenotrophomonas maltophilia, Stenotrophomonas nitritireducens, Stenotrophomonas rhizophila; Streptobacillus species: Streptobacillus moniliformis (Streptobacillary rat bite fever); Streptococcus species: Streptococcus Group A, Streptococcus Group B, Streptococcus agalactiae, Streptococcus aginosus, Streptococcus avium, Streptococcus bovis, Streptococcus canis, Streptococcus cricetus, Streptococcus faceium, Streptococcus faecalis, Streptococcus ferus, Streptococcus gallinarum, Streptococcus lactis, Streptococcus milleri, Streptococcus mitior, Streptococcus mitis, Streptococcus mutans, Streptococcus oralis, Streptococcus peroris, Streptococcus pneumoniae, Streptococcus pyogenes, Streptococcus ratti, Streptococcus salivarius, Streptococcus sanguinis, Streptococcus sobrinus, Streptococcus parasanguinis, Streptococcus suis, Streptococcus thermophilus, Streptococcus vestibularis, Streptococcus viridans, Streptococcus uberis, Streptococcus zooepidemicus; Tatumlla species, Trabulsiella species, Treponema species: Treponema carateum (Pinta), Treponema denticola, Treponema endemicum (Bejel), Treponema pallidum (Syphilis), Treponema pertenue (Yaws); Tropheryma whipplei (Whipple disease), Tuberculoid leprosy, Ureaplasma urealyticum, Veillonella, Vibrio species: Vibrio aerogenes, Vibrio aestuarianus, Vibrio agarivorans, Vibrio albensis, Vibrio alginolyticus, Vibrio brasiliensis, Vibrio calviensis, Vibrio campbellii, Vibrio chagasii, Vibrio cholerae (Cholera), Vibrio cincinnatiensis, Vibrio comma, Vibrio coralliilyticus, Vibrio crassostreae, Vibrio cyclitrophicus, Vibrio diabolicus, Vibrio diazotrophicus, Vibrio ezurae, Vibrio fischeri, Vibrio fluvialis, Vibrio fortis, Vibrio furnissii, Vibrio gallicus, Vibrio gazogenes, Vibrio gigantis, Vibrio halioticoli, Vibrio harveyi, Vibrio hepatarius, Vibrio hispanicus, Vibrio ichthyoenteri, Vibrio kanaloae, Vibrio lentus, Vibrio litoralis, Vibrio logei, Vibrio mediterranei, Vibrio metschnikovii, Vibrio mimicus, Vibrio mytili, Vibrio natriegens, Vibrio navarrensis, Vibrio neonatus, Vibrio neptunius, Vibrio nereis, Vibrio nigripulchritudo, Vibrio ordalii, Vibrio orientalis, Vibrio pacinii, Vibrio parahaemolyticus, Vibrio pectenicida, Vibrio penaeicida, Vibrio pomeroyi, Vibrio ponticus, Vibrio proteolyticus, Vibrio rotiferianus, Vibrio ruber, Vibrio rumoiensis, Vibrio salmonicida, Vibrio scophthalmi, Vibrio splendidus, Vibrio superstes, Vibrio tapetis, Vibrio tasmaniensis, Vibrio tubiashii, Vibrio vulnificus, Vibrio wodanis, Vibrio xuii; Vogesella indigofera, Wigglesworthia species, Wolbachia species, Xenorhabdus species, Yersinia enterocolitica, Yersinia pestis, Yersinia pseudotuberculosis, and Yokenella species.
61. The method according to items 39 and 60, wherein the vira is selected from the group consisting of Abelson murine leukemia virus (Ab-MLV, A-MuLV), acute laryngotracheobronchitis virus (or HPIV), Adelaide River virus, Adeno-associated virus group (Dependevirus), Adenovirus, African horse sickness virus, African swine fever virus, AIDS virus, Aleutian mink disease, parvovirus, alfalfa mosaic virus, Alphaherpesvirinae (including HSV 1 and 2 and varicella), Alpharetrovirus (Avian leukosis virus, Rous sarcoma virus), Alphavirus, alkhurma virus, ALV related virus, Amapari virus, Andean potato mottle virus, Aphthovirus, Aquareovirus, arbovirus, arbovirus C, arbovirus group A, arbovirus group B, Arenavirus group, Argentine hemorrhagic fever virus, Argentinian hemorrhagic fever virus, Arterivirus, Astrovirus, Ateline herpesvirus group, Aujezky's disease virus, Aura virus, Ausduk disease virus, Australian bat lyssavirus, Aviadenovirus, avian erythroblastosis virus, avian infectious bronchitis virus, avian leukemia virus, Avian leukosis virus (ALV), avian lymphomatosis virus, avian myeloblastosis virus, avian paramyxovirus, avian pneumoencephalitis virus, avian reticuloendotheliosis virus, avian sarcoma virus, avian type C retrovirus group, Avihepadnavirus, Avipoxvirus, B virus (Cercopithecine herpesvirus 1), B19 virus (Parvovirus B19), Babanki virus, baboon herpesvirus, bacterial virus, baculovirus, barley yellow dwarf virus, Barmah Forest virus, bean pod mottle virus, bean rugose mosaic virus, Bebaru virus, Berrimah virus, Betaherpesvirinae, betaretrovirus, Bird flu, Birnavirus, Bittner virus, BK virus, Black Creek Canal virus, bluetongue virus, Bolivian hemorrhagic fever virus, Boma disease virus, border disease of sheep virus, borna virus, bovine alphaherpesvirus 1, bovine alphaherpesvirus 2, bovine coronavirus, bovine ephemeral fever virus, bovine immunodeficiency virus, bovine leukemia virus, bovine leukosis virus, bovine mammillitis virus, bovine papillomavirus, bovine papular stomatitis virus, bovine parvovirus, bovine syncytial virus, bovine type C oncovirus, bovine viral diarrhea virus, bracovirus, broad bean mottle virus, broad bean stain virus, brome mosaic virus, Bromovirus, Buggy Creek virus, bullet shaped virus group, Bunyamwera virus, Bunyavirus, Burkitt's lymphoma virus, Bwamba Fever, Bwattany hetero virus, CA virus (Croup-associated virus/parainfluenza vius type 2), Calicivirus, California encephalitis virus, camelpox virus, canarypox virus, canid herpesvirus, canine coronavirus, canine distemper virus, canine herpesvirus, canine minute virus, canine parvovirus, Cano Delgadito virus, Capillovirus, caprine arthritis virus, caprine encephalitis virus, Caprine Herpes Virus, Capripox virus, Cardiovirus, Carlavirus, Carmovirus, carrot mottle virus, Cassia yellow blotch virus, Caulimovirus, Cauliflower mosaic virus, caviid herpesvirus 1, Cercopithecine herpesvirus 1, Cercopithecine herpesvirus 2, cereal yellow dwarf virus, Chandipura virus, Changuinola virus, channel catfish virus, Charleville virus, chickenpox virus, Chikungunya virus, chimpanzee herpesvirus, Chordopoxyirinae, chub reovirus, chum salmon virus, Closterovirus, Cocal virus, Coho salmon reovirus, coital exanthema virus, Colorado tick fever virus, Coltivirus Columbia SK virus, Commelina yellow mottle virus, Common cold virus, Comovirus, Condylomata accuminata, congenital cytomegalovirus, contagious eethyma virus, contagious pustular dermatitis virus, Coronavirus, Corriparta virus, coryza virus, cowpea chlorotic mottle virus, cowpea mosaic virus, cowpea virus, cowpox virus, coxsackie virus, CPV (cytoplasmic polyhedrosis virus), cricket paralysis virus, Crimean-Congo hemorrhagic fever virus, croup associated virus, Crypotovirus, Cucumovirus, Cypovirus, Cytomegalovirus (HCMV or Human Herpesvirus 5 HHV-5), cytoplasmic polyhedrosis virus, Cytorhabdovirus, deer papillomavirus, Deltaretrovirus (Human T-lymphotropic virus), Deformed wing virus DWV, Dengue, Densovirus, Dependovirus, Dhori virus, Dianthovirus, diploma virus, DNA virus, Dobrava-Belgrade Virus, Dog Flu, Drosophila C virus, duck hepatitis B virus, duck hepatitis virus 1, duck hepatitis virus 2, duovirus, Duvenhage virus, eastern equine encephalitis virus, eastern equine encephalomyelitis virus, Ebola virus, Ebola-like virus, Echovirus, echovirus 10, echovirus 28, echovirus 9, ectromelia virus, EEE virus (Eastern equine encephalitis virus), EIA virus (equine infectious anemia), EMC virus (Encephalomyocarditis), Emiliania huxleyi virus 86, encephalitis virus, encephalomyocarditis virus, Endogenous retrovirus, Enterovirus, Entomopoxyirinae, Entomopoxvirus A, Entomopoxvirus B, Entomopoxvirus C, enzyme elevating virus, epidemic hemorrhagic fever virus, epizootic hemorrhagic disease virus, Epsilonretrovirus, Epstein-Barr virus (EBV; Human herpesvirus 4 HHV-4), equid alphaherpesvirus 1, equid alphaherpesvirus 4, equid herpesvirus 2, equine abortion virus, equine arteritis virus, equine encephalosis virus, equine infectious anemia virus, equine morbillivirus, equine rhinopneumonitis virus, equine rhinovirus, Eubenangu virus, European elk papillomavirus, European swine fever virus, Everglades virus, Eyach virus, Fabavirus, felid herpesvirus 1, feline calicivirus, feline fibrosarcoma virus, feline herpesvirus, feline immunodeficiency virus, feline infectious peritonitis virus, feline leukemia/sarcoma virus, feline leukemia virus, feline panleukopenia virus, feline parvovirus, feline sarcoma virus, feline syncytial virus, Fijivirus, Filovirus, Flanders virus, Flavivirus, foot and mouth disease virus, Fort Morgan virus, Four Corners hantavirus, fowl adenovirus 1, Fowlpox virus, Friend virus, Furovirus, Gammaherpesvirinae, gammaretrovirus, GB virus C(GBV-C; formerly Hepatitis G virus), Geminivirus, German measles virus, Getah virus, gibbon ape leukemia virus, green monkey virus (mullburg), glandular fever virus, goatpox virus, golden shinner virus, Gonometa virus, goose parvovirus, granulosis virus, Gross' virus, ground squirrel hepatitis B virus, group A arbovirus, Guanarito virus, guinea pig cytomegalovirus, guinea pig type C virus, Hantavirus, hard clam reovirus, hare fibroma virus, HCMV (human cytomegalovirus), helper virus, hemadsorption virus 2, hemagglutinating virus of Japan, hemorrhagic fever virus, Hendra virus, Henipaviruses, Hepadnavirus, hepatitis A virus, hepatitis B virus, hepatitis C virus, hepatitis D (delta) virus, hepatitis E virus, hepatitis F virus, hepatitis G virus, hepatitis nonA nonB virus, hepatoencephalomyelitis reovirus 3, Hepatovirus, heron hepatitis B virus, herpes B virus, Herpes simplex virus, herpes simplex virus 1, herpes simplex virus 2, Herpesvirus, Herpes zoster, Herpes virus 6, Herpes virus 7, Herpes virus 8, Herpesvirus ateles, Herpesvirus hominis, Herpesvirus saimiri, Herpesvirus suis, Herpesvirus varicellae, Highlands J virus, Hirame rhabdovirus, HIV-1, hog cholera virus, Hordeivirus, Horse Flu, HTLV-1, HTLV-2, human adenovirus 2, human alphaherpesvirus 1, human alphaherpesvirus 2, human alphaherpesvirus 3, human B lymphotropic virus, human betaherpesvirus 5, human coronavirus, Human enterovirus A, Human enterovirus B, Human Flu, human foamy virus, human gammaherpesvirus 4, human gammaherpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus (HIV), human immunodeficiency virus 1, human immunodeficiency virus 2, Human metapneumovirus, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lymphotropic virus type 2, human T lymphotropic virus I, human T lymphotropic virus II, human T lymphotropic virus III, ichnovirus, Ilarvirus, infantile gastroenteritis virus, infectious bovine rhinotracheitis virus, infectious haematopoietic necrosis virus, infectious pancreatic necrosis virus, influenza virus, influenzavirus A, influenzavirus B, influenzavirus C, influenzavirus D, influenzavirus pr8, insect iridescent virus, insect virus, interfering virus, iridovirus, Isavirus, Japanese B virus, Japanese encephalitis virus, JC virus, Junin virus, Johnson grass mosaic virus, Kaposi's sarcoma-associated herpesvirus, Kemerovo virus, Kilham's rat virus, Klamath virus, Kolongo virus, Korean hemorrhagic fever virus, kumba virus, Kumlinge virus, Kunjin virus, Kyasanur forest disease, Kyzylagach virus, La Crosse virus, lactic dehydrogenase elevating virus, Lagos bat virus, Lambda phage, langat virus, Langur virus, lapine parvovirus, Lassa fever virus, Lassa virus, latent rat virus, LCM virus, Leaky virus, Lentivirus, Leporipoxvirus, leukemia virus, leukovirus, louping ill virus, lumpy skin disease virus, Luteovirus, lymphadenopathy associated virus, Lymphocytic choriomeningitis virus (LCMV), Lymphocryptovirus, lymphocytic choriomeningitis virus, lymphoproliferative virus group, Lyssavirus, Machupo virus, mad itch virus, maize chlorotic dwarf virus, maize rough dwarf virus, mammalian type B oncovirus group, mammalian type B retroviruses, mammalian type C retrovirus group, mammalian type D retroviruses, mammary tumor virus, Mapuera virus, Marafivirus, Marburg virus, Marburg-like virus, Mason Pfizer monkey virus, Mastadenovirus, Mayaro virus, ME virus, Measles virus, Melandrium yellow fleck virus, Menangle virus, Mengo virus, Mengovirus, Merkel cell polyomavirus, Middelburg virus, milkers nodule virus, mink enteritis virus, minute virus of mice, MLV related virus, MM virus, Mokola virus, Molluscipoxvirus, Molluscum contagiosum virus, Moloney murine leukemia virus (M-MuLV), monkey B virus, Monkeypox virus, Mononegavirales, Morbillivirus, Mount Elgon bat virus, mouse cytomegalovirus, mouse encephalomyelitis virus, mouse hepatitis virus, mouse K virus, mouse leukemia virus, mouse mammary tumor virus, mouse minute virus, mouse pneumonia virus, mouse poliomyelitis virus, mouse polyomavirus, mouse sarcoma virus, mousepox virus, Mozambique virus, Mucambo virus, mucosal disease virus, Mumps virus, murid betaherpesvirus 1, murid cytomegalovirus 2, murine cytomegalovirus group, murine encephalomyelitis virus, murine hepatitis virus, murine leukemia virus, murine nodule inducing virus, murine polyomavirus, murine sarcoma virus, Muromegalovirus, Murray Valley encephalitis virus, myxoma virus, Myxovirus, Myxovirus multiforme, Myxovirus parotitidis, Nairobi sheep disease virus, Nairovirus, Nanirnavirus, Nariva virus, Ndumo virus, Necrovirus, Neethling virus, Nelson Bay virus, Nemtick Virus, Nepovirus, neurotropic virus, New World Arenavirus, newborn pneumonitis virus, Newcastle disease virus, Nipah virus, noncytopathogenic virus, Norovirus, Norwalk virus, nuclear polyhedrosis virus (NPV), nipple neck virus, O'nyong'nyong virus, oat sterile dwarf virus, Ockelbo virus, Omsk hemorrhagic fever virus, oncogenic virus, oncogenic viruslike particle, oncornavirus, Orbivirus, Orf virus, Oropouche virus, Orthohepadnavirus, orthomyxovirus, Orthopoxvirus, Orthoreovirus, Orungo, ovine papillomavirus, ovine catarrhal fever virus, owl monkey herpesvirus, Palyam virus, Papillomavirus, Papillomavirus sylvilagi, Papovavirus, Parainfluenza virus human (HPIV), parainfluenza virus type 1 human (HPIV-1), parainfluenza virus type 2 human (HPIV-2), parainfluenza virus type 3 human (HPIV-3), parainfluenza virus type 4 human (HPIV-4), Paramyxovirus, Parapoxvirus, paravaccinia virus, parsnip yellow fleck virus, Parvovirus, Parvovirus B19, pea enation mosaic virus, Pestivirus, Phlebovirus, phocine distemper virus, Phytoreovirus, Picodnavirus, Picornavirus, pig cytomegalovirus, pigeonpox virus, Pity virus, Pixuna virus, plant rhabdovirus group, plant virus, pneumonia virus of mice, Pneumovirus, Poliomyelitis virus, Poliovirus, Polydnavirus, polyhedral virus, Polyoma virus, Polyomavirus, Polyomavirus bovis, Polyomavirus cercopitheci, Polyomavirus hominis 2, Polyomavirus maccacae 1, Polyomavirus muris 1, Polyomavirus muris 2, Polyomavirus papionis 1, Polyomavirus papionis 2, Polyomavirus sylvilagi, Pongine herpesvirus 1, porcine epidemic diarrhea virus, porcine hemagglutinating encephalomyelitis virus, porcine parvovirus, porcine transmissible gastroenteritis virus, porcine type C virus, Potato leaf roll virus, Potato mop top virus, Potato virus Y, Potexvirus, Potyvirus, Powassan encephalitis virus, Poxvirus, poxvirus variolae, Prospect Hill virus, provirus, pseudocowpox virus, pseudorabies virus, psittacinepox virus, Puumala virus, Qalyub virus, Quail pea mosaic virus, quailpox virus, Queensland fruitfly virus, Quokkapox virus, rabbit fibroma virus, rabbit kidney vaculolating virus, rabbit papillomavirus, Rabies virus, raccoon parvovirus, raccoonpox virus, radish mosaic virus, Ranikhet virus, rat cytomegalovirus, rat parvovirus, rat virus, Rauscher's virus, recombinant vaccinia virus, recombinant virus, Red Clover Necrotic Mosaic Virus, reovirus, reovirus 1, reovirus 2, reovirus 3, reptilian type C virus, Respiratory syncytial virus, respiratory virus, reticuloendotheliosis virus, Retrovirus, Rhabdovirus, Rhabdovirus carpia, Rhadinovirus, Rhinovirus, Rhizidiovirus, rice dwarf virus, rice gall dwarf virus, rice hoja Blanca virus, rice ragged stunt virus, Rift Valley fever virus, Riley's virus, rinderpest virus, RNA tumor virus, RNA virus, Roseolovirus, Ross River virus, Rotavirus, rougeole virus, Rous sarcoma virus, Rubella virus, rubeola virus, Rubivirus, Russian autumn encephalitis virus, S6-14-03 virus, SA 11 simian virus, SA 15, SA2 virus, SA6 virus, SA8 virus, Sabia virus, Sabio virus, Sabo virus, Saboya virus, Sabulodes caberata GV, Sacbrood virus, Saccharomyces cerevisiae virus L-A, Saccharomyces cerevisiae virus La, Saccharomyces cerevisiae virus LBC, Sagiyama virus, Saguaro cactus virus, Saimiriine herpesvirus 1, Saimiriine herpesvirus 2, Sainpaulia leaf necrosis virus, SaintAbb's Head virus, Saint-Floris virus, Sakhalin virus, Sal Vieja virus, Salanga virus, Salangapox virus, Salehabad virus, salivary gland virus, Salmonid herpesvirus 1, Salmonid herpesvirus 2, Salmonis virus, Sambucus vein clearing virus, Samia cynthia NPV, Samia pryeri NPV, Samia ricini NPV, Sammons' Opuntia virus, SanAngelo virus, San Juan virus, San Miguel sealion virus, San Perlita virus, Sand rat nuclear inclusion agents, Sandfly fever Naples virus, Sandfly fever Sicilian virus, Sandjimba virus, Sango virus, Santa Rosa virus, Santarem virus, Santosai temperate virus, Sapphire II virus, Sapporo-like virus, Saraca virus, Sarracenia purpurea virus, SARS virus, satellite virus, Sathuperi virus, Satsuma dwarf virus, Saturnia pavonia virus, Saturnia pyri NPV, Saumarez Reef virus, Sawgrass virus, Sceliodes cordalis NPV, Schefflera ringspot virus, Sciaphila duplex GV, Scirpophaga incertulas NPV, Sciurid herpesvirus, Sciurid herpesvirus 2, Scoliopteryx libatFix NPV, Scopelodes contracta NPV, Scopelodes venosa NPV, Scopula subpunctaria NPV, Scotogramma trifolii GV, Scotogramma trifolu NPV, Scrophularia mottle virus, SDAV (sialodacryoadenitis virus), sealpox virus, Selenephera lunigera NPV, Selepa celtis GV, Seletar virus, Selidosema suavis NPV, Semidonta biloba NPV, Semiothisa sexmaculata GV, Semliki Forest Virus, Sena Madureira virus, Sendai virus, SENV-D, SENV-H, Seoul virus, Sepik virus, Serra do Navio virus, Serrano golden mosaic virus, Sesame yellow mosaic virus, Sesamia calamistis NPV, Sesamia cretica GV, Sesamia inferens NPV, Sesamia nonagrioides GV, Setora nitens virus, Shallot latent virus, Shamonda virus, Shark River virus, Sheep associated malignant catarrhal fever, Sheep papillomavirus, Sheep pulmonary adenomatosis associated herpesvirus, sheeppox virus, Shiant Islands virus, Shokwe virus, Shope fibroma virus, Shope papilloma virus, Shuni virus, Siamese cobra herpesvirus, Sibine fusca densovirus, Sida golden mosaic virus (SiGMV), Sida golden yellow vein virus (SiGYVV), Sigma virus, Sikte water-borne virus, Silverwater virus, Simbu virus, Simian adenoviruses 1 to 27, Simian agent virus 12, Simian enterovirus 1 to 18, simian foamy virus, Simian hemorrhagic fever virus, simian hepatitis A virus, simian human immunodeficiency virus, simian immunodeficiency virus, simian parainfluenza virus, Simian rotavirus SA11, Simian sarcoma virus, simian T cell lymphotrophic virus, Simian type D virus 1, Simian vancella herpesvirus, simian virus, simian virus 40, Simplexvirus, Simulium vittatum densovirus, Sin Nombre virus, Sindbis virus, Sint1em's onion latent virus, Sixgun city virus, Skunkpox virus, Smallpox virus, Smelt reovirus, Smerinthus ocellata NPV, Smithiantha virus, Snakehead rhabdovirus, Snowshoe hare virus, Snyder-Theilen feline sarcoma virus, Sobemovirus, Sofyn virus, Soil-borne wheat mosaic virus, Sokoluk virus, Solanum apical leaf curl virus, Solanum nodiflorum mottle virus, Solanurn yellows virus, Soldado virus, Somerville virus 4, Sonchus mottle virus, Sonchus virus, Sonchus yellow net virus, Sorghum chlorotic spot virus, Sorghum mosaic virus, Sorghum virus, Sororoca virus, Soursop yellow blotch virus, SouthAfrican passiflora virus, South American hemorrhagic fever viruses, SouthAfrican passiflora virus, South River virus, Southern bean mosaic virus, Southern potato latent virus, Sowbane mosaic virus, Sowthistle yellow vein virus, Soybean chlorotic mottle virus, Soybean crinkle leaf virus, Soybean dwarf virus, Soybean mosaic virus, SPAr-2317 virus, Sparganothis pettitana NPV, sparrowpox virus, Spartina mottle virus, Spectacled caimanpox virus, SPH 114202 virus, Sphenicid herpesvirus 1, Sphinx ligustri NPV, Spider monkey herpesvirus, Spilarctia subcarnea NPV, Spilonota ocellana NPV, Spilosoma lubricipeda NPV, Spinach latent virus, Spinach temperate virus, Spiroplasma phage 1, Spiroplasma phage 4, Spiroplasma phage aa, Spiroplasma phage C1/TS2, Spodoptera exempta cypovirus, Spodoptera exigua virus, Spodoptera frugiperda virus, Spodoptera latifascia virus, Spodoptera littoralis, Spodoptera mauritia virus, Spodoptera ornithogalli virus, Spondweni virus, spring beauty latent virus, Spring viremia of carp virus, Spumavirus (SFV, HFV), Squash leaf curl virus, squash mosaic virus, squirrel fibroma virus, Squirrel monkey herpesvirus, squirrel monkey retrovirus, SR-11 virus, Sri Lankan passionfruit mottle virus, Sripur virus, SSV 1 virus group, StAbbs Head virus, St. Louis encephalitis virus, Staphylococcus phage 107, Staphylococcus phage 187, Staphylococcus phage 2848A, Staphylococcus phage 3A, Staphylococcus phage 44A HJD, Staphylococcus phage 77, Staphylococcus phage B11-M15, Staphylococcus phage Twort, Starlingpox virus, Statice virus Y, P, STLV (simian T lymphotropic virus) type I, STLV (simian T lymphotropic virus) type II, STLV (simian T lymphotropic virus) type Ill, stomatitis papulosa virus, Stratford virus, Strawberry crinkle virus, Strawberry latent ringspot virus, Strawberry mild yellow edge virus, Strawberry vein banding virus, Streptococcus phage 182, Streptococcus phage 2BV, Streptococcus phage A25, Streptococcus phage 24, Streptococcus phage PE1, Streptococcus phage VD13, Streptococcus phage fD8, Streptococcus phage CP-1, Streptococcus phage Cvir, Streptococcus phage H39, Strigid herpesvirus 1, Striped bass reovirus, Striped Jack nervous, necrosis virus, Stump-tailed macaque virus, submaxillary virus, Subterranean clover mottle virus, Subterranean clover mottle virus satellite, Subterranean clover red leaf virus, Subterranean clover stunt virus, Sugarcane bacilliform virus, Sugarcane mild mosaic virus, Sugarcane mosaic virus, Sugarcane streak virus, suid alphaherpesvirus 1, suid herpesvirus 2, Suipoxvirus, Sulfolobus virus 1, Sunday Canyon virus, Sunflower crinkle virus, Sunflower mosaic virus, Sunflower rugose mosaic virus, Sunflower yellow blotch virus, Sunflower yellow ringspot virus, Sun-hemp mosaic virus, swamp fever virus, Sweet clover necrotic mosaic virus, Sweet potato A virus, Sweet potato chlorotic leafspot virus, Sweet potato feathery mottle virus, Sweet potato internal cork virus, Sweet potato latent virus, Sweet potato mild mottle virus, Sweet potato russet crack virus, Sweet potato vein mosaic virus, Sweet potato yellow dwarf virus, Sweetwater Branch virus, Swine cytomegalovirus, Swine Flu, Swine infertility and respiratory syndrome virus, swinepox virus, Swiss mouse leukemia virus, Sword bean distortion mosaic virus, Synaxis jubararia NPV, Synaxis pallulata NPV, Synetaeris tenuifemur virus, Syngrapha selecta NPV, T4 phage, T7 phage, TAC virus, Tacaiuma virus, Tacaribe complex virus, Tacaribe virus, Tadpole edema virus LT 1-4, Taggert virus, Tahyna virus, Tai virus, Taiassui virus, Tamana bat virus, Tamarillo mosaic virus, Tamdy virus, [[Tamiami virus, Tanapox virus, Tanga virus, Tanjong Rabok virus, Taro bacilliform virus, Badnavirus Tataguine virus, Taterapox virus, Taterapox virus, Teasel mosaic virus, Tehran virus, Telfairia mosaic virus, Telok Forest virus, Tembe virus, Tembusu virus, Tench reovirus, Tensaw virus, Tenvivirus, Tephrosia symptomless virus, Termeil virus, Tete virus, Tetralopha scortealis NPV, Tetropium cinnamoptemm NPV, Texas pepper virus, Thailand virus, Thaumetopoea pityocampa virus, Theiler's encephalomyelitis virus, Theiler's virus, Theophila mandarina NPV, Theretra japonica NPV, Thermoproteus virus 1, Thermoproteus virus 2, Thermoproteus virus 3, Thermoproteus virus 4, Thiafora virus, Thimiri virus, Thistle mottle virus, Thogoto virus, Thormodseyjarklettur virus, Thosea asigna virus, Thosea baibarana NPV, Thosea sinensis GV, Thottapalayam virus, Thylidolpteryx ephemeraeformis NPV, Thymelicus lineola NPV, Tibrogargan virus, Ticera castanea NPV, Tick borne encephalitis virus (TBEV)—European and Far Eastern subtypes, Tillamook virus, Tilligerry virus, Timbo virus, Tilmboteua virus, Tilmaroo virus, Tindholmur virus, Tinea pellionella NPV, Tineola hisselliella NPV, Tinpula paludosa NPV, Tinracola plagiata NPV, Tioman virus, Tlacotalpan virus, Tobacco bushy top virus, Tobacco etch virus, Tobacco leaf curl virus, Tobacco mild green mosaic virus, tobacco mosaic virus, Tobacco mosaic virus satellite, Tobacco mottle virus, Tobacco necrosis virus, Tobacco necrosis virus satellite, Tobacco necrosis virus small satellite, Tobacco necrotic dwarf virus, tobacco rattle virus, Tobacco ringspot virus, Tobacco streak virus, Tobacco stunt virus, Tobacco vein banding mosaic virus, Tobacco vein distorting virus Tobacco vein mottling virus, Tobacco wilt virus, Tobacco yellow dwarf virus, Tobacco yellow net virus, Tobacco yellow vein virus, Tobamovirus Tobravirus, Togavirus, Tomato apical stunt viroid, Tomato aspermy virus, Tomato black ring virus, Tomato black ring virus satellite, Tomato bunchy top viroid, tomato bushy stunt virus, Tomato bushy stunt virus satellite, Tomato golden mosaic virus, Tomato leaf crumple virus, Tomato leaf curl virus, Tomato leafroll virus, Tomato mosaic virus, Tomato mottle virus, Tomato pale chlorosis virus, Tomato planta macho viroid, Tomato pseudo-curly top virus, Tomato ringspot virus, Tomato spotted wilt virus, Tomato top necrosis virus, Tomato vein yellowing virus, Tomato yellow dwarf virus, Tomato yellow leaf curl virus, Tomato yellow mosaic virus, Tomato yellow top virus, Tombusvirus, Tongan vanilla virus, Torovirus, Torque teno virus, Tortrix loeflingiana NPV, Tortrix viridana NPV, Toscana virus, Tospovirus, Toxorhynchites brevipalpis NPV, Trabala vishnou NPV, Tradescantia/Zebrina virus, Trager duck spleen necrosis virus, Tranosema sp. Virus, transforming virus, Tree shrew adenovirus 1, Tree shrew herpesvims, Triatoma virus, Tribec virus, Trichiocampus irregularis NPV, Trichiocampus viminalis NPV, Trichomonas vaginalis virus, Trichoplusia ni cypovirus 5, Trichoplusia ni granulovirus, Trichoplusia ni MNPV, Trichoplusia ni Single SNPV, Trichoplusia ni virus, Trichosanthes mottle virus, Triticum aestivum chlorotic spot virus, Trivittatus virus, Trombetas virus, Tropaeolum virus 1, Tropaeolum virus 2, Trubanarnan virus, Tsuruse virus, Tucunduba virus, Tulare apple mosaic virus, Tulip band breaking virus, Tulip breaking virus, Tulip chlorotic blotch virus, Tulip top breaking virus, Tulip virus X, tumor virus, Tupaia virus, Tupaiid herpesvirus 1, Turbot herpesvirus, Turbot reovirus, Turkey adenoviruses 1 to 3, Turkey coronavirus, Turkey herpesvirus 1, turkey rhinotracheitis virus, turkeypox virus, Turlock virus, Turnip crinkle virus, Turnip crinkle virus satellite, Turnip mild yellows virus, Turnip mosaic virus, Turnip rosette virus, turnip yellow mosaic virus, Turuna virus, Tymovirus, Tyuleniy virus, type C retroviruses, type D oncovirus, type D retrovirus group, Uasin Gishu disease virus, Uganda S virus, Ugymyia sericariae NPV, ulcerative disease rhabdovirus, Ullucus mild mottle virus, Ullucus mosaic virus, Ullucus virus C, Umatilla virus, Umbre virus, Una virus, Upolu virus, UR2 sarcoma virus, Uranotaenia sapphirina NPV, Urbanus proteus NPV, Urucuri virus, Ustilago maydis virus 1, Ustilago maydis virus 4, Ustilago maydis virus 6, Usutu virus, Uting a virus, Utive virus, Uukuniemi virus group, Vaccinia virus, Vaeroy virus, Vallota mosaic virus, Vanessa atalanta NPV, Vanessa cardui NPV, Vanessa prorsa NPV, Vanilla mosaic virus, Vanilla necrosis virus, Varicella zoster virus, Varicellovirus, Varicola virus, variola major virus, variola virus, Vasin Gishu disease virus, Vellore virus, Velvet tobacco mottle virus, Velvet tobacco mottle virus satellite, Venezuelan equine encephalitis virus, Venezuelan equine encephalomyelitis virus, Venezuelan hemorrhagic fever virus, Vesicular stomatitis virus, Vesiculovirus, Vibrio phage 06N-22P, Vibrio phage 06N-58P, Vibrio phage 4996, Vibrio phage a3a, Vibrio phage I, Vibrio phage II, Vibrio phage m, Vibrio phage IV, Vibrio phage kappa, Vibrio phage nt-1, Vibrio phage OXN-52P, Vibrio phage OXN-IOOP, Vibrio phage v6, Vibrio phage Vfl2, Vibrio phage Vf33, Vibrio phage VP1, Vibrio phage VP11, Vibrio phage VP3, Vibrio phage VP5, Vibrio phage X29, Vicia cryptic virus, Vigna sinensis mosaic virus, Vilyuisk virus, Vinces virus, Viola mottle virus, viper retrovirus, viral haemorrhagic septicemia virus, virus-like particle, Visna Maedi virus, Visna virus, Voandzeia mosaic virus, Voandzeia necrotic mosaic virus, volepox virus, Wad Medani virus, Wallal virus, Walleye epidermal hyperplasia, Walrus calicivirus, Wanowrie virus, Warrego virus, Watermelon chlorotic stunt virus, Watermelon curly mottle virus, Watermelon mosaic virus 1, Watermelon mosaic virus 2, Weddel water-borne virus, Weldona virus, Wesselsbron virus, West Nile virus, western equine encephalitis virus, western equine encephalomyelitis virus, Wexford virus, Whataroa virus, Wheat American striate mosaic virus, Wheat chlorotic streak virus, Wheat dwarf virus, Wheat rosette stunt virus, Wheat streak mosaic virus, Wheat yellow leaf virus, Wheat yellow mosaic virus, White bryony virus, White clover cryptic virus 1, White clover cryptic virus 2, White clover cryptic virus 3, White clover mosaic virus, White lupinrnosaic virus, Wild cucumber mosaic virus, Wild potato mosaic virus, Wildbeest herpesvirus, Wineberry latent virus, Winter wheat mosaic virus, Winter wheat Russian mosaic virus, Wiseana cervinata virus, Wiseana signata virus, Wiseana umbraculata virus, Wissadula mosaic virus, Wisteria vein mosaic virus, Witwatersrand virus, Wongal virus, Wongorr virus, Winter Vomiting Virus, woodchuck hepatitis B virus, Woodchuck herpesvirus marmota 1, woolly monkey sarcoma virus, wound tumor virus, WRSV virus, WVU virus 2937, WW virus 71 to 212, Wyeomyia smithii NPV, Wyeomyia virus, Xanthomonas phage Cf, Xanthomonas phage Cflt, Xanthomonas phage RR66, Xanthomonas phage Xf, Xanthomonas phage Xf2, Xanthomonas phage XP5, Xenopus virus T21, Xiburema virus, Xingu virus, Xylena curvimacula NPV, Y73 sarcoma virus, Yaba monkey tumor virus, Yaba-1 virus, Yaba-7 virus, Yacaaba virus, Yam mosaic virus, Yaounde virus, Yaquina Head virus, Yatapoxvirus, Yellow fever virus, Yogue virus, Yokapox virus, Yokase virus, Yponomeuta cognatella NPV, Yponomeuta evonymella NPV, Yponomeuta malinellus NPV, Yponomeuta padella NPV, Yucca baciliform virus, Yug Bogdanovac virus, Zaliv Terpeniya virus, Zea mays virus, Zegla virus, Zeiraphera diniana virus, Zeiraphera pseudotsugana NPV, Zika virus, Zirqa virus, Zoysia mosaic virus, Zucchini yellow fleck virus, Zucchini yellow mosaic virus, and Zygocactus virus.
62. A polynucleotide probe suitable for interaction with a complementary RNA and/or DNA target, wherein the polynucleotide probe comprises exactly 1 intercalator molecule.
63. A polynucleotide probe suitable for interaction with a complementary RNA and/or DNA target, wherein the polynucleotide probe comprises at least 2 intercalator molecules.
64. The polynucleotide probe according to any of items 63 and 64, wherein the polynucleotide probe can be selected from the group consisting of a DNA probe, a RNA probe, a LNA probe and a PNA probe.
65. The polynucleotide probe according to any of items 63 to 65, wherein the total number of intercalator molecules can be selected from the group consisting of 2, 3, 4, 5, 6, 7, 8, 9, 10, 11, 12, 13, 14, 15, 16, 17, 18, 19, 20 and more than 20 intercalator molecules.
66. The polynucleotide probe according to any of items 63 to 66, wherein the insertion of the intercalator molecules results in increased melting point of a polynucleotide duplex consisting of the target DNA and/or RNA and the complementary probe.
67. The polynucleotide probe according to any of items 63 to 67, wherein the ratio between the number of intercalator molecules and the total number of bases the polynucleotide probe is from 1:50 to 1:2 such as from 1:50 to 1:40, for example 1:40 to 1:30, such as from 1:30 to 1:20, for example 1:20 to 1:10, such as from 1:10 to 1:5, for example 1:5 to 1:2, or any combination of these intervals.
68. The polynucleotide probe according to any of items 63 to 68, wherein the one and only intercalator molecule is selected from the group consisting of TINA, INA, ortho-TINA, para-TINA, AMANY.
69. A polynucleotide probe according to item 69 wherein the intercalator molecule is TINA.
70. A polynucleotide probe according to item 69 wherein the intercalator molecule is INA.
71. A polynucleotide probe according to item 69 wherein the intercalator molecule is ortho-TINA.
72. A polynucleotide probe according to item 69 wherein the intercalator molecule is para-TINA.
73. A polynucleotide probe according to item 69 wherein the intercalator molecule is AMANY.
74. The polynucleotide probe according to any of items 63 to 69, wherein the two or more intercalator molecules can be selected from the group consisting of TINA, INA, ortho-TINA, para-TINA, AMANY.
75. The polynucleotide probe according to any of items 63 to 69, wherein the polynucleotide probe comprises more than one type of intercalator molecules such as 2, 3, 4, 5 or more than 5 different types of intercalator molecules.
76. The polynucleotide probe according to any of items 63 to 76, wherein the polynucleotide probe is connected to a support.
77. The polynucleotide probe according to item 77, wherein the support is selected from the group consisting of particulate matters, beads, magnetic beads, non-magnetic beads, polystyrene beads, magnetic polystyrene beads, sepharose beads, sephacryl beads, polystyrene beads, agarose beads, polysaccharide beads, and polycarbamate beads.
78. The polynucleotide probe according to any of items 77-78, wherein the support is a solid support.
79. The polynucleotide probe according to item 79, wherein the solid support can be selected from the group consisting of microtiter plate or other plate formats, reagent tubes, glass slides or other supports for use in array or microarray analysis, tubings or channels of micro fluidic chambers or devices and Biacore chips.
80. The polynucleotide probe according to any of items 63 to 76, wherein the polynucleotide probe comprises one or more labels.
81. The polynucleotide probe according to item 81, wherein the one or more labels can be selected from the group consisting of biotin, a fluorescent label, 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, dyes, Cy2, Cy3, and Cy5, PerCP, phycobiliproteins, R-phycoerythrin (RPE), allophycoerythrin (APC), Texas Red, Princeston Red, Green fluorescent protein (GFP) and analogues thereof, conjugates of R-phycoerythrin or allophycoerythrin, inorganic fluorescent labels based on semiconductor nanocrystals (like quantum dot and Qdot™ nanocrystals), time-resolved fluorescent labels based on lanthanides like Eu3+ and Sm3+, haptens, DNP, digoxiginin, enzymic labels, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase and glucose oxidase (GO), luminiscence labels, luminol, isoluminol, acridinium esters, 1,2-dioxetanes, pyridopyridazines, radioactivity labels, isotopes of iodide, isotopes of cobalt, isotopes of elenium, isotopes of tritium, and isotopes of phosphor.
82. The polynucleotide probe according to any of items 63 to 76, wherein the intercalating unit of the intercalator preferably comprises a chemical group selected from the group consisting of polyaromates and heteropolyaromates.
83. The polynucleotide probe according to item 83, wherein the polyaromates or heteropolyaromates consist of at least 2 aromatic rings, such as 3, for example 4, such as 5, for example 6, such as 7, for example 8, such as more than 8 aromatic rings.
84. The polynucleotide probe according to items 83-84, wherein the heteropolyaromates contains at least one aromatic ring wherein at least one carbon atom is replaced by a heteroatom selected from nitrogen and oxygen.
85. The polynucleotide probe according to items 83-84, Wherein the heteropolyaromates contains at least 2 heteroatoms, such as 3 heteroatoms, for example 4 heteroatoms, such as 5 heteroatoms, for example more than 5 heteroms.
86. The polynucleotide probe according to items 83-84 and 86, wherein the heteropolyaromates contains oxygen as the only heteroatoms.
87. The polynucleotide probe according to items 83-84 and 86, wherein the heteropolyaromates contains nitrogen as the only heteroatom.
88. The polynucleotide probe according to items 83-84 and 86, wherein the heteropolyaromates contains both nitrogen and oxygen as heteroatom.
89. The polynucleotide probe according to items 83-84, wherein the polyaromates or heteropolyaromates are substituted with one or more substituents selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl and amido.
90. The polynucleotide probe according to any of items 83-90, wherein the intercalating unit of the intercalator is selected from the group consisting of polyaromates and heteropolyaromates that are capable of increasing the stability of the polynucleotide duplex structure.
91. The polynucleotide probe according to any of items 84-91, wherein the intercalating unit of the intercalator is selected from the group consisting of phenanthroline, phenazine, phenanthridine, pyrene, anthracene, naphthalene, phenanthrene, picene, chrysene, naphtacene, benzanthracene, stilbene, porphyrin and any of the aforementioned intercalators substituted with one or more substituents selected from the group consisting of hydroxyl, halogen, mercapto, thio, cyano, alkylthio, heterocycle, aryl, heteroaryl, carboxyl, carboalkoyl, alkyl, alkenyl, alkynyl, nitro, amino, alkoxyl and amido.

### EXAMPLES

### Evaluation of Abasic Sites on Tm by Melting Curve Acquisition

A Fluorescence Resonance Energy Transfer (FRET) system on the LightCycler® 2.0 was used to evaluate Melting point (Tm) changes induced by base mismatches or abasic sites (B) in two different oligopolynucleotide sequences.

Oligonucleotides were purchased from IBA GmbH (Göttingen, Germany) or DNA Technology A/S (Risskov, Denmark) on a 0.2 μmol synthesis scale with high performance liquid chromatography (HPLC) purification and subsequently quality control. Oligonucleotides were synthesized with a 3′ amino-modifier-C7 and thereafter linked to ATTO495 NHS-ester or a 5′-amino-modifier-C6 and thereafter linked to an ATTO590 NHS-ester. ATTO495 functions as FRET donor and is a modification of Acridine Orange with excitation maximum at 495 nm and emission maximum at 527 nm. ATTO590 is a derivative of Rhodamine dyes with excitation maximum at 594 nm and emission maximum at 624 nm. The ATTO495/ATTO590 FRET pair was excitated at 470 nm on a LightCycler®2.0 (Roche Applied Science, Basel, Switzerland) and fluorescence emission was detected at 640 nm.

For evaluation of Uracil-DNA Glycosylase (UNG) treatment of uracil containing oligonucleotides before Tm determination a preincubation step was performed. 10 μM oligonucleotide was incubated in 20 mM Tris-HCl (pH 8.2 at 25° C.), 10 mM NaCl, 1 mM EDTA with or without 1 Unit of UNG per 50 pmol oligonucleotide at 37° C. for 1 hour.

Melting curve experiments were performed on a LightCycler® 2.0 using 20 μL LightCycler capillaries. 1.0 μM of each oligonucleotide was mixed with sodium phosphate buffer (50 mM NaH₂PO₄/Na₂HPO₄, 100 mM NaCl and 0.1 mM EDTA) at pH 7.0. Tm measurements were carried out using a standard program of (i) a dissociation step from 37 to 95° C. with a ramp rate of 0.2° C./second and hold for 5 minutes at 95° C., (ii) annealing from 95 to 37° C. with a ramp rate of 0.05° C./second and continued measurement of fluorescence, (iii) hold at 37° C. for 5 minutes and (iv) denaturation from 37 to 95° C. with a ramp rate of 0.05° C./second and continued measurement of fluorescence. The fluorescence data for both the annealing and denaturation curves were used for Tm determination and no hysteresis was observed. Tm was identified using the LightCycler Software 4.1 for melting curve analysis and defined as the peak of the first derivate. All melting curve determinations were conducted as single capillary measurements. Prior to Tm identification, runs were color compensated by subtraction of the fluorophore background fluorescence.

Results are presented in Table 1 to 3. A single abasic site decreased Tm by 10° C. in average (Table 1). For oligonucleotides with two mismatches or two abasic sites (Table 2) we observed an average decrease in Tm by 15.7° C. of two mismatches (7.8° C./mismatch) compared with an average decrease in Tm of 23.8° C. by two abasic sites (11.9° C./abasic site). Abasic sites in average decreased Tm 52% more than mismatch bases ((11.9−7.8)/7.8*100).

TABLE 1
Effect of a single abasic site on Tm

| Effect of a single abasic | | | | |
|---|---|---|---|---|
| site on Tm | | | | |
| (LightCycler determina- | | | | |
| tion of Tm in 50 mM | | | | |
| phosphate buffer with 100 | | | | |
| mM NaCI and 0.1 mM EDTA | | | | |
| at pH 7.0. B equals a | | LC D-821 | LC D-822 | LC D-823 |
| stable abasic site. Tm | | 5′ TGGGGAGCATAACAGGATT- | 5′ TGGGGAGCAUAACAGGATT- | 5′ TGGGGAGCABAACAGGATT- |
| in ° C.) | | ATTO495 3′ | ATTO495 3′ | ATTO495 3′ |
| LC D-824 | 5′ ATTO590- | 68.2 | 68.2 | 59.0 |
| | AATCCTGTTATGCTCCCCA | | | |
| | 3′ | | | |
| LC D-825 | 5′ ATTO590- | 57.7 | 58.0 | 57.7 |
| | AATCCTGTTBTGCTCCCCA | | | |
| | 3′ | | | |

TABLE 2
Effect of two abasic sites compared with base mismatches on Tm.

| Effect of a two abasic | | | | | |
|---|---|---|---|---|---|
| site on Tm and effect | | | | | |
| of UNG treatment on | | | | | |
| uracil containing | | | | | |
| oligonucleotides | | | | | |
| (LightCycler deter- | | | | | |
| mination of Tm in 50 | | | | | |
| mM phosphate buffer | | | | | |
| with 100 mM NaCl | | LC D-826 | LC D-827 | | |
| and 0.1 mM EDTA at | | 5′ | 5′ | LC D-828 | LC D-829 |
| pH 7.0. B equals a | | TTAGGGTTTAG | TTAGGGATTAG | 5′ | 5′ |
| stable abasic site. | | GGTTTAGGG- | GGATTAGGG- | TTAGGGUTTAGGGUTTAGGG- | TTAGGGBTTAGGGBTTAGGG- |
| Tm in ° C.) | | ATTO495 3′ | ATTO495 3′ | ATTO495 3′ | ATTO495 3′ |
| LC | 5′ ATTO590- | 67.5 | 53.5 | 69.6 | 44.4 |
| D- | CCCTAAACCCTAAACCCTAA | | | | |
| 830 | 3′ | | | | |
| LC | 5′ ATTO590- | 51.6 | 67.7 | 52.3 | 40.9 |
| D- | CCCTAATCCCTAATCCCTAA | | | | |
| 831 | 3′ | | | | |
| LC | 5′ ATTO590- | 51.5 | 66.8 | 52.4 | 41.7 |
| D- | CCCTAAUCCCTAAUCCCTAA | | | | |
| 832 | 3′ | | | | |
| LC | 5′ ATTO590- | 44.5 | 45.4 | 47.3 | 44.5 |
| D- | CCCTAABCCCTAABCCCTAA | | | | |
| 833 | 3′ | | | | |

Table 3 shows the effect of pre-incubation of uracil containing oligonucleotides with or without UNG treatment. Without UNG treatment an average decrease in Tm by 17.4° C. of two mismatches (8.7° C./mismatch) compared with an average decrease in Tm of 21.3° C. by two UNG treated uracil bases (10.6° C./UNG treated Uracil).

TABLE 3
Effect of pre-incubation of uracil containing oliognucleotides with
UNG. Non-UNG treated uracil containing oligonucleotides and UNG or
non-UNG treated oligonucleotides with abasic (B) sites as controls.

| | | | | |
|---|---|---|---|---|
| Uracil or abasic site con- | | | | |
| taining oligonucleotides | | | | |
| and effet of UNG treatment | | | | |
| on Tm (LightCycler | LC D-828 | LC D-828 | LC D-829 | LC D-829 |
| determination of Tm in | 5′ | 5′ | 5′ | 5′ |
| 50 mM phosphate buffer | TTAGGGUTTAG | TTAGGGUTTAG | TTAGGGBTTAGG | TTAGGGBTTAG |
| with 100 mM NaCl and | GGUTTAGGG- | GGUTTAGGG- | GBTTAGGG- | GGBTTAGGG- |
| 0.1 mM EDTA at pH 7.0. | ATTO495 | ATTO495 | ATTO495 | ATTO495 |
| B equals a stable abasic | 3′ NO UNG | 3′ UNG treated | 3′ No UNG | 3′ UNG |
| site. Tm in ° C.) | treatment | (abasic) | treatment | treated |

| | | | | | |
|---|---|---|---|---|---|
| LC | 5′ ATTO590- | 69.7 | 49.8 | 44.8 | 45.6 |
| D- | CCCTAAACCCTAAACCCTAA | | | | |
| 830 | 3′ | | | | |
| LC | 5′ ATTO590- | 52.2 | 45.6 | 41.2 | 41.7 |
| D- | CCCTAATCCCTAATCCCTAA | | | | |
| 831 | 3′ | | | | |
| LC | 5′ ATTO590- | 52.5 | 50.1 | 42.3 | 45.4 |
| D- | CCCTAAUCCCTAAUCCCTAA | | | | |
| 832 | 3′ | | | | |
| LC | 5′ ATTO590- | 48.1 | 49.8 | 44.8 | 45.4 |
| D- | CCCTAABCCCTAABCCCTAA | | | | |
| 833 | 3′ | | | | |

In conclusion we have shown that abasic sites destabilize the oligonucleotide hybridization 52% more efficiently than base mismatches. Furthermore we have shown that the effect of abasic sites compared with base mismatch sites is not only present for stable abasic sites, but also UNG treated Uracil bases.

UNG Treatment of Uracil Containing Target and Specific Capture with Para-TINA Containing Oligonucleotides or Conventional DNA Oligonucleotides

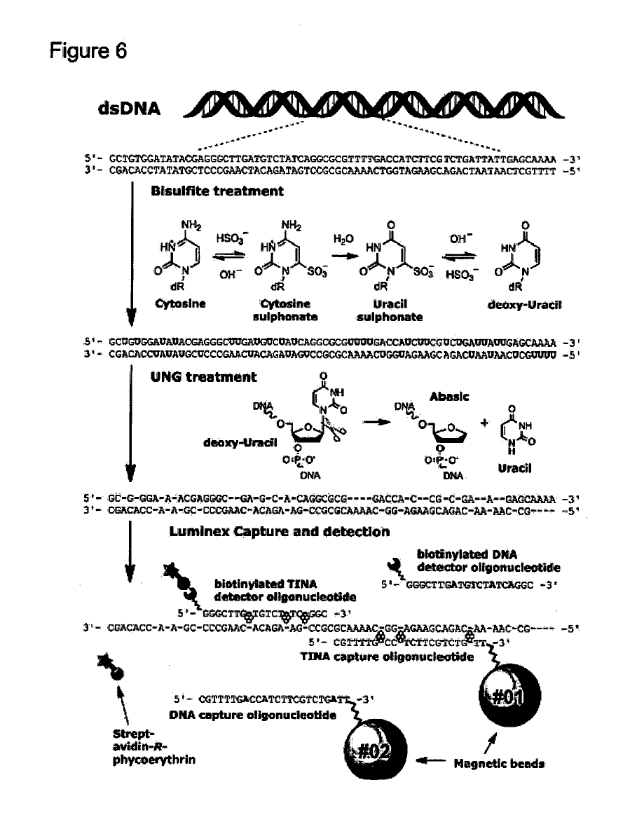

The aim of this experiment was to remove a specific base from dsDNA and thereafter to capture the DNA by oligonucleotides in which the complementary base had been substituted with para-TINA (FIGS. 6 & 7).

Oligonucleotides were purchased from IBA GmbH (Göttingen, Germany) or DNA Technology A/S (Risskov, Denmark) on a 0.2 pmol synthesis scale with high performance liquid chromatography (HPLC) purification and subsequently quality control. The STX2 gene base pair 230 to 300 from Escherichia coli was used as target sequence. The oligonucleotides were STX2 230-300F: 5′-GCUGUGGAUAUACGAGGGCUUGAUGUCUAUCAGGCGCGUUUUGACCAUCUUC GUCUGAUUAUUGAGCAAAA-3′ and STX2 230-300R: 5′-UUUUGCUCAAUAAUCAGACGAAGAUGGUCAAAACGCGCCUGAUAGACAUCAAGC CCUCGUAUAUCCACAGC-3′, where Thymines were substituted by deoxy-Uracil (dU). The capture oligonucleotides were either a conventional DNA oligonucleotide STX2-A003C: 5′-CGTTTTGACCATCTTCGTCTGATTAA-HEX-CX—NH₂-3′ or a para-TINA oligonucleotide STX2-A004C: 5′-CGTTTTGXCCXTCTTCGTCTGXTTAA-HEX-CX—NH₂-3′. HEG is a Hexaethylene glycol spacer, CX—NH₂ is an aminomodified cyclohexane spacer and X is para-TINA. Detection was done using either a conventional DNA oligonucleotide STX2-A001B: bio-GGGCTTGATGTCTATCAGGC-3′ or a para-TINA oligonucleotide STX2-A002B: bio-GGGCTTGXTGTCTXTCXGGC-3′. Bio- is a C6-biotin spacer and X is para-TINA.

Preincubation and UNG Treatment of dsDNA

For formation of dsDNA 1.00 pmol of STX2 230-300R was mixed with 1.60 pmol STX2 230-300F in 20 mM Tris-HCl (pH 8.2 at 25° C.), 10 mM NaCl, 1 mM EDTA and heated to 95° C. for 5 minutes. Reannealing was done at 60° C. for 15 minutes followed by 15 minutes at 25° C. When relevant 1 Unit of Uracil-DNA Glycosylase was added and incubated at 37° C. for 1 hour. UNG treated dsDNA was diluted from 1.0*10̂-12 mol/well in 10-fold dilution to 1.0*10̂-18 mol/well in 20 mM Tris-HCl (pH 8.2 at 25° C.), 10 mM NaCl and 1 mM EDTA.

### Coupling of Oligonucleotides to Luminex MagPlex™ Microspheres

The conventional DNA capture oligonucleotide (STX2-A003C) was coupled to MagPlex™-C Magnetic carboxylated microspheres following the recommendations from Luminex Corp. In short 2.5×10⁶ microspheres were activated in 0.1 M MES, pH 4.5, added 0.2 nmole oligonucleotide and 25 μg EDC. The coupling reaction was incubated for 30 minutes in the dark and added 25 μg EDC and incubated for 30 minutes again. 1.0 mL of 0.02% Tween-20 was added and the supernatant was removed after magnetic separation for 1 minute on a DynaMag™-2 Magnetic Particle Concentrator (Invitrogen, Tåstrup, Denmark). 1 mL of 0.1% SDS was added and vortexed followed by magnetic separation and resuspended in 100 μL Tris-EDTA buffer, pH 8.0 and stored in the refrigerator.

The para-TINA modified oligonucleotide (STX2-A004C) was coupled using a novel in-house carbodiimide/sulpho-NHS coupling procedure. 2.5×10⁶ microspheres were transferred to a low retention microcentrifuge tube (Axygen, Union City, Calif., USA). Microspheres were washed and activated in 100 μL of 0.1 M MES, pH 6.0 followed by resuspension in 35 μL buffer. 125 μg supho-NHS was added followed by 625 μg EDC. Microspheres were incubated in the dark for 15 minutes and added 625 μg EDC followed by incubation for 15 minutes again. Activation buffer was removed and 97 μL of 0.1 M phosphate buffer, pH 7.2 added followed by 0.3 nmol oligonucleotide. Microspheres were incubated on a Thermo-shaker TS-100 (BioSan, Riga, Latvia) at 900 rpm for 2 hours at room temperature. Incubation was continued over night (optional) without shaking. Microspheres were washed once in 100 μL of 0.1 M phosphate buffer, pH 7.2 followed by blocking in 0.1 M phosphate buffer with 50 mM ethanolamine, pH 7.2 and incubation for 15 minutes at 900 rpm at room temperature on the Thermo-shaker TS-100. Microspheres were separated and resuspended in 100 μL Tris-EDTA buffer, pH 8.0 and stored at 5° C. All separation steps were done by placing the microcentrifuge tube in the magnetic separator for 1 minute and tubes were vortexed at low speed for 20 seconds after each addition of buffer or reagent.

To ensure equal coupling efficiency for both the carbodiimide and the carbodiimide/sulpho-NHS coupling procedures, a biotinylated oligonucleotide with or without para-TINA was included in each coupling protocol. The coupling efficiency was evaluated by incubation of 0.2 μL microspheres with 0.5 μg Streptavidin-R-PhycoErythrin Prem. Grad (S-21388, Invitrogen A/S, Tåstrup, Denmark) with 10 μg Albumin fraction V (Merck & Co Inc.), 0.03% Triton X-100 and 10 mM phosphate buffer, pH 6.4 with 200 mM NaCl. The reaction mixture was incubated for 15 minutes at 25° C. and 900 rpm in an iEMS@ Incubator/Shaker HT (Thermo Fisher Scientific). Washed three times in the 10 mM phosphate buffer, pH 6.4 with 200 mM NaCl and 0.03% Triton X-100 followed by counting of 350 microspheres on the Luminex®200™ instrument. Similar coupling efficiencies were found using both procedures.

Luminex Detection of UNG Treated dsDNA

0.2 μL of STX2-A003C or STX2-A004C beads were mixed in a 96 MicroWell™ Plate with conical bottom shape (NUNC, Thermo Fisher Scientific, Roskilde, Denmark) with 1.0 pmol/well of STX2-A001B or STX2-A002B as detection oligo in 50 mM NaH₂PO₄/Na₂HPO₄, pH 7.0 with 100 mM NaCl, 0.1 mM EDTA and 0.03% Triton X-100 and added UNG or non-UNG treated dsDNA in 10-fold dilution from 1.0*10̂-12 mol/well to 1.0*10̂-18 mol/well. The mixture was incubated at 69° C. for 10 minutes in an iEMS® Incubator/Shaker HT (Thermo Fisher Scientific) at 900 rpm and thereafter at 35° C. for 15 minutes at 900 rpm. After incubation the plate was washed three times by placing the plate in a 96-well magnetic separator (PerkinElmer, Skovlunde, Denmark) and removing the supernatant followed by addition of 12.5 mM NaH₂PO₄/Na₂HPO₄ with 25 mM NaCl, 25 μM EDTA and 0.03% Triton X-100 at pH 7.0. Each well was added 0.5 μg Streptavidin-R-PhycoErythrin Prem. Grad (S-21388, Invitrogen A/S, Tåstrup, Denmark) with 10 μg Albumin fraction V (Merck & Co Inc.) in 50 mM NaH₂PO₄/Na₂HPO₄, pH 7.0 with 100 mM NaCl, 0.1 mM EDTA and 0.03% Triton X-100. The plate was incubated for 15 minutes in the iEMS® Incubator/Shaker HT at 35° C. at 900 rpm. After incubation the plate was washed three times as previously explained. 12.5 mM NaH₂PO₄/Na₂HPO₄ with 25 mM NaCl, 25 μM EDTA and 0.03% Triton X-100 at pH 7.0 was added and the plates was incubated for 30 minutes at 35° C. before analysis on the Luminex200™ instrument, counting 150 of each microsphere set. The final step at 35° C. was necessary to avoid decreasing background fluorescence throughout the Luminex analysis due to sedimentation of uneven sized microspheres (Hanley B P, Xing L, Cheng R H (2007) Variance in multiplex suspension array assays: microsphere size variation impact. Theor Biol Med Model 4:31).

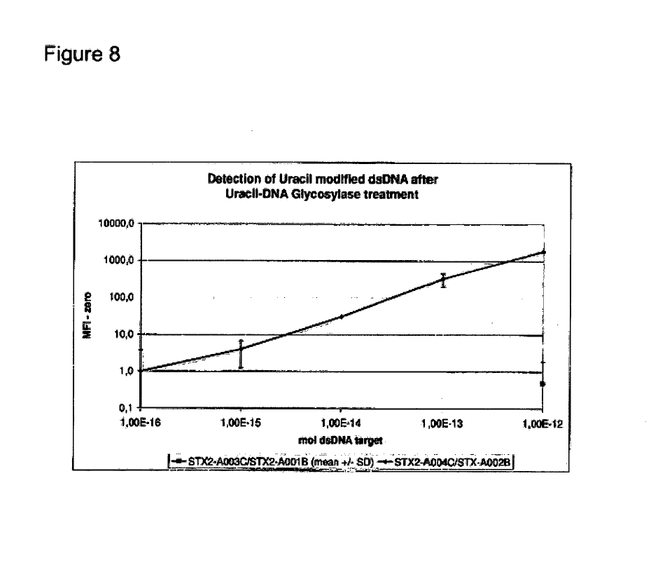

Table 4 and FIG. 8 compare the Luminex readings from non-UNG and UNG treated dsDNA in duplicates. For conventional DNA oligonucleotides without UNG treatment (Table 4 column one and two) we find an increase in MFI by increasing concentration of dsDNA, since a minor portion of the dsDNA will be denatured at an incubation temperature of 69° C. This increase in signal is removed by UNG treatment (Table 4 column three and four) indicating that the deoxy-Uracil bases have been cleaved from the dsDNA leaving abasic site to which the conventional DNA oligonucleotides are not able to anneal at the stringent buffer conditions. For para-TINA containing oligonucleotides no signal is observed for dsDNA without UNG treatment (Table 4 column five and six), which is expected since the para-TINA is placed direct opposite a deoxy-Uracil base. After UNG treatment a liniar concentration dependent increase in MFI is observed for para-TINA containing oligonucleotides (Table 4 column seven and eight and FIG. 8), which is expected due to the removal of deoxy-Uracil by UNG leaving abasic sites and decreasing annealing temperature for the dsDNA helix.

TABLE 4
Raw Data as Median Fluorescence Intensity -
MFI from the Luminex system.

| | | Conventional | | | | para-TINA | | | |
|---|---|---|---|---|---|---|---|---|---|
| | | DNA | | | | modified | | | |
| | UNG | oligonucleatides | | | | oligonucleotides | | | |
| | treated | NO | NO | YES | YES | NO | NO | YES | YES |
| mol | 0 | 2 | 2 | 3 | 2 | 10 | 10 | 10 | 10 |
| dsDNA | 1.00E−18 | 7 | 6 | 3 | 2 | 13 | 11 | 10 | 10 |
| | 1.00E−17 | 9 | 7 | 3 | 2 | 11 | 11 | 11 | 10 |
| | 1.00E−16 | 10 | 7 | 2 | 2 | 13 | 11 | 12 | 10 |
| | 1.00E−15 | 14 | 10 | 2 | 2 | 11 | 11 | 15 | 13 |
| | 1.00E−14 | 34 | 28 | 2 | 2 | 12 | 13 | 41 | 41 |
| | 1.00E−13 | 95 | 84 | 3 | 2 | 13 | 15 | 297 | 390 |
| | 1.00E−12 | 32 | 27 | 3 | 3 | 10 | 11 | 1819 | 1869 |

In conclusion we have shown that deoxy-Uracil bases can be cleaved from dsDNA leaving destabilized dsDNA that after denaturation can be used for annealing to oligonucleotides where the adenine bases have been substituted by para-TINA making the intercalator a specific base in the annealing to the DNA strand.

Effect on Melting Point (Tm) of Nucleobase Mismatches and Abasic Sites in the Probe when Placed Opposite Unmodified Target Sequence and the Effect of Nucleobase Mismatches, Abasic Sites and Ortho-TINA Molecules in the Probe, when they are Placed Opposite Abasic Sites in the Oligonucleotide Target Sequence

A Fluorescence Resonance Energy Transfer (FRET) system on the LightCycler® 2.0 was used to evaluate melting point (Tm) changes induced by nucleobase mismatches or abasic sites (B) in the probe. Likewise target oligonucleotides with abasic sites were evaluated in hybridization with complementary probe oligonucleotides containing abasic sites, ortho-TINA molecules or natural DNA nucleobases.

Oligonucleotides were purchased from Eurofins (Ebersberg, Germany) on a 0.2 pmol synthesis scale. The oligonucleotides were synthesized on an ABI-3900 with reverse phase high performance liquid chromatography (RP-HPLC) purification and a final quality control by mass spectrometry analysis before lyophilization. Oligonucleotides were redissolved in double-distilled water to a stock concentration of 100 μM and left overnight at 5° C. before use. Oligonucleotides were synthesized with a 3′ amino-modifier-C7 and thereafter linked to ATTO647N NHS-ester or a 5′-amino-modifier-C6 and thereafter linked to an ATTO488 NHS-ester. The ATTO488/ATTO647N FRET pair was excitated at 470 nm on a LightCycler®2.0 (Roche Applied Science, Basel, Switzerland) and fluorescence emission was detected at 670 nm.

Melting curve experiments were performed on a LightCycler® 2.0 using 20 μL LightCycler capillaries. 1.0 μM of each oligonucleotide was mixed with sodium phosphate buffer (50 mM NaH₂PO₄/Na₂HPO₄, 100 mM NaCl and 0.1 mM EDTA) at pH 7.0. Tm measurements were carried out using a standard program of (i) a dissociation step from 37 to 95° C. with a ramp rate of 0.2° C./second and hold for 5 minutes at 95° C., (ii) annealing from 95 to 37° C. with a ramp rate of 0.05° C./second and continued measurement of fluorescence, (iii) hold at 37° C. for 5 minutes and (iv) denaturation from 37 to 95° C. with a ramp rate of 0.05° C./second and continued measurement of fluorescence. The fluorescence data for both the annealing and denaturation curves were used for Tm determination and no hysteresis was observed. Tm was identified by the classical Tm determination method by identifying the starting fluorescence plateau and the final fluorescence plateau. The Tm was determined at the median fluorescence between these two plateaus. All melting curve determinations were conducted as single capillary measurements. Prior to Tm identification, runs were color compensated by subtraction of the fluorophore background fluorescence in the buffer used for the experiments.

Results are presented in Table 4 to 13.

Table 4 to 8 show the effect on Tm of one to three nucleobase mismatches or abasic sites in the probe sequence. In general, it is observed that central nucleobase mismatches decrease Tm more than nucleobase mismatches towards the ends of the oligonucleotides. Likewise decreasing the length of the oligonucleotides in the duplex from 30 down to 18 nucleotides decreases the Tm of the duplex, but increases the change in Tm by nucleobase mismatches, thus following conventional rules regarding mismatches, oligo lengths and ΔTm. In all cases, abasic sites in the probe decrease Tm substantially more compared to nucleobase mismatches.

For a 30-mer duplex probe with a single nucleobase mismatch, the average Tm decreased by 5.9° C. (range 2.8° C. to 7.8° C.) compared to Tm for matching oligonucleotides. The average decrease in Tm by an abasic site was 9.6° C. (range 8.3° C. to 11.9° C.) or an additional decrease of 63% for an abasic site compared with a single nucleobase mismatch (some of the data are presented in Table 4 and 5). Likewise, for a 22-mer duplex, the average Tm decrease by a single nucleobase mismatch was 8.5° C. (range 4.3° C. to 13.0° C.) compared to 12.9° C. (range 12.8° C. to 12.9° C.) for an abasic site or an additional decrease of 52% induced by abasic site instead of single nucleobase mismatch.

TABLE 4

| Effect of a single nucleo- | | | | | | | | | | |
|---|---|---|---|---|---|---|---|---|---|---|
| base mismatch or abasic | | | | | | | | | | |
| site in probe on Tm. | | | | | | | | | | |
| LightCycler 2.0 deter- | | | | | | | | | | |
| mination of Tm (° C.) in | | | | | | | | | | |
| 50 mM phosphate buffer | | | | | | | | | | |
| with 100 mM NaCl and 0.1 | | | | | | | | | | |
| mM EDTA at pH 7.0. The | | | rrs_ | rrs_ | rrs_ | | rrs_ | rrs_ | rrs_ | |
| nucleobase for which the | | rrs_ | 1341- | 1341- | 1341- | | 1341- | r1341- | 1341- | |
| complementary nucleobase | | 1341- | 12_064 | 12_071 | 12_078 | | 12_064 | 12_071 | 12_078 | |
| is altered is highlighted | | 12_049 | A | T | C | rrs_ | A | T | C | rrs_ |
| in underlined bold. The | | G | nucleo- | nucleo- | nucleo- | 1341- | nucleo- | nucleo- | nucleo- | 1341- |
| change in Tm towards Tm for | | nucleo- | tide | tide | tide | 12_085 | tide | tide | tide | 12_085 |
| the match base pairs (ΔTm) | | tide | mis- | mis- | mis- | Abasic | mis- | mis- | mis- | Abasic |
| are shown to the right. | | match | match | match | match | site | match | match | match | site |
| rrs_ | ATTO488- | 76.1 | 72.8 | 72.3 | 73.3 | 67.8 | −3.3 | −3.8 | −2.8 | −8.3 |
| 1312- | TATTGCACAATGGGCGCAAGCC | | | | | | | | | |
| 41_001 | TGATGCAG | | | | | | | | | |
| rrs_ | ATTO488- | 75.4 | 71.5 | 70.9 | 72.4 | 66.9 | −3.9 | −4.5 | −3.0 | −8.5 |
| 1312- | TATTGCACAATGGGCGCAAGCC | | | | | | | | | |
| 39_002 | TGATGC | | | | | | | | | |
| rrs_ | ATTO488- | 72.5 | 66.1 | 66.0 | 66.2 | 62.4 | −6.4 | −6.5 | −6.3 | −10.1 |
| 1312- | TATTGCACAATGGGCGCAAGCC | | | | | | | | | |
| 37_003 | TGAT | | | | | | | | | |
| rrs_ | ATTO488- | 71.4 | 63.5 | 64.1 | 63.4 | 60.3 | −7.9 | −7.3 | −8.0 | −11.1 |
| 1312- | TATTGCACAATGGGCGCAAGCC | | | | | | | | | |
| 35_004 | TG | | | | | | | | | |
| rrs_ | ATTO488- | 70.3 | 62.8 | 61.3 | 61.1 | 57.5 | −7.5 | −9.0 | −9.2 | −12.8 |
| 1312- | TATTGCACAATGGGCGCAAGCC | | | | | | | | | |
| 33_005 | | | | | | | | | | |
| rrs_ | ATTO488- | 68.6 | 59.7 | 58.9 | 59.4 | 54.4 | −8.9 | −9.7 | −9.2 | −14.2 |
| 1312- | TATTGCACAATGGGCGCAAG | | | | | | | | | |
| 31_006 | | | | | | | | | | |
| rrs_ | ATTO488- | 66.8 | 56.2 | 55.4 | 54.6 | 50.8 | −10.6 | −11.4 | −12.2 | −16.0 |
| 1312- | TATTGCACAATGGGCGCA | | | | | | | | | |
| 29_007 | | | | | | | | | | |

TABLE 5

| Effect of a single nucleo- | | | | | | | | | | |
|---|---|---|---|---|---|---|---|---|---|---|
| base mismatch or abasic | | | | | | | | | | |
| site in probe on Tm. | | | | | | | | | | |
| LightCycler 2.0 deter- | | | | | | | | | | |
| mination of Tm (° C.) in | | | | | | | | | | |
| 50 mM phosphate buffer | | | | | | | | | | |
| with 100 mM NaCl and 0.1 | | | | | | | | | | |
| mM EDTA at pH 7.0. The | | | rrs_ | rrs_ | rrs_ | | rrs_ | rrs_ | rrs_ | |
| nucleobase for which the | | rrs_ | 1341- | 1341- | 1341- | | 1341- | 1341- | 1341- | |
| complementary nucleobase | | 1341- | 12_065 | 12_072 | 12_079 | | 12_065 | 12_072 | 12_079 | |
| is altered is highlighted | | 12_049 | A | T | C | rrs_ | A | T | C | rrs_ |
| in underlined bold. The | | G | nucleo- | nucleo- | nucleo- | 1341- | nucleo- | nucleo- | nucleo- | 1341- |
| change in Tm towards Tm for | | nucleo- | tide | tide | tide | 12_086 | tide | tide | tide | 12_086 |
| the match base pairs (ΔTm) | | tide | mis- | mis- | mis- | Abasic | mis- | mis- | mis- | Abasic |
| are shown to the right. | | match | match | match | match | site | match | match | match | site |
| rrs_ | ATTO488- | 76.1 | 68.3 | 68.8 | 68.5 | 67.6 | −7.8 | −7.3 | −7.6 | −8.5 |
| 1312- | TATTGCACAATGGGCGCAAGCC | | | | | | | | | |
| 41_001 | TGATGCAG | | | | | | | | | |
| rrs_ | ATTO488- | 75.4 | 66.8 | 67.6 | 67.5 | 66.2 | −8.6 | −7.8 | −7.9 | −9.2 |
| 1312- | TATTGCACAATGGGCGCAAGCC | | | | | | | | | |
| 39_002 | TGATGC | | | | | | | | | |
| rrs_ | ATTO488- | 72.5 | 62.3 | 63.3 | 63.1 | 61.8 | −10.2 | −9.2 | −9.4 | −10.7 |
| 1312- | TATTGCACAATGGGCGCAAGCC | | | | | | | | | |
| 37_003 | TGAT | | | | | | | | | |
| rrs_ | ATTO488- | 71.4 | 60.5 | 61.3 | 60.9 | 59.7 | −10.9 | −10.1 | −10.5 | −11.7 |
| 1312- | TATTGCACAATGGGCGCAAGCC | | | | | | | | | |
| 35_004 | TG | | | | | | | | | |
| rrs_ | ATTO488- | 70.3 | 57.3 | 59.3 | 58.9 | 57.4 | −13.0 | −11.0 | −11.4 | −12.9 |
| 1312- | TATTGCACAATGGGCGCAAGCC | | | | | | | | | |
| 33_005 | | | | | | | | | | |
| rrs_ | ATTO488- | 68.6 | 56.3 | 58.5 | 57.2 | 55.6 | −12.3 | −10.1 | −11.4 | −13.0 |
| 1312- | TATTGCACAATGGGCGCAAG | | | | | | | | | |
| 31_006 | | | | | | | | | | |
| rrs_ | ATTO488- | 66.8 | 54.0 | 55.7 | 54.2 | 53.3 | −12.8 | −11.1 | −12.6 | −13.5 |
| 1312- | TATTGCACAATGGGCGCA | | | | | | | | | |
| 29_007 | | | | | | | | | | |

Table 4 and Table 5. Effect on Tm by a single nucleobase mismatch or an abasic site determined for oligonucleotides with lengths from 18 to 30 nucleotides.

For a 30-mer duplex probe with two nucleobase mismatches, the average Tm decreased by 13.2° C. (range 10.5° C. to 16.5° C.) compared to Tm for matching oligonucleotide probes. The average decrease in Tm by two abasic sites was 21.6° C. (range 19.3° C. to 23.6° C.) or an additional decrease of 64% for two abasic sites compared to two nucleobase mismatches (some of the data are presented in Table 6 and 7). Likewise for a 22-mer duplex probe, the average Tm decrease by two nucleobase mismatches was 18.2° C. (range 13.4° C. to 25.3° C.) compared to above 20.9° C. for two abasic sites (range 19.9° C. to beyond the limits of the Tm determination on the LightCycler 2.0).

TABLE 6

| Effect of two nucleobase | | | | | | | | | | |
|---|---|---|---|---|---|---|---|---|---|---|
| mismatches or abasic sites | | | | | | | | | | |
| in probe on Tm. LightCycler | | | | | | | | | | |
| 2.0 determination of Tm | | | | | | | | | | |
| (° C.) in 50 mM phosphate | | | | | | | | | | |
| buffer with 100 mM NaCl and | | | | | | | | | | |
| 0.1 mM EDTA at pH 7.0. The | | | rrs_ | rrs_ | rrs_ | | rrs_ | rrs_ | rrs_ | |
| nucleobases for which the | | rrs_ | 1341- | 1341- | 1341- | | 1341- | 1341- | 1341- | |
| complementary nucleobases | | 1341- | 12_068 | 12_075 | 12_082 | | 12_068 | 12_075 | 12_082 | |
| are altered are highlighted | | 12_049 | A | T | C | rrs_ | A | T | C | rrs_ |
| in underlined bold. The | | G- | nucleo- | nucleo- | nucleo- | 1341- | nucleo- | nucleo- | nucleo- | 1341- |
| change in Tm towards Tm for | | nucleo- | tide | tide | tide | 12_089 | tide | tide | tide | 12_089 |
| the match base pairs (ΔTm) | | tide | mis- | mis- | mis- | Abasic | mis- | mis- | mis- | Abasic |
| are shown to the right. | | matches | matches | matches | matches | sites | matches | matches | matches | sites |
| rrs_ | ATTO488- | 76.1 | 65.5 | 63.8 | 62.6 | 56.8 | −10.6 | −12.3 | −13.5 | −19.3 |
| 1312- | TATTGCACAATGGGCGCAAGCC | | | | | | | | | |
| 41_001 | TGATGCAG | | | | | | | | | |
| rrs_ | ATTO488- | 75.4 | 63.8 | 62.1 | 60.5 | 54.7 | −11.6 | −13.3 | −14.9 | −20.7 |
| 1312- | TATTGCACAATGGGCGCAAGCC | | | | | | | | | |
| 39_002 | TGATGC | | | | | | | | | |
| rrs_ | ATTO488- | 72.5 | 59.2 | 57.2 | 54.2 | 50.0 | −13.3 | −15.3 | −18.3 | −22.5 |
| 1312- | TATTGCACAATGGGCGCAAGCC | | | | | | | | | |
| 37_003 | TGAT | | | | | | | | | |
| rrs_ | ATTO488- | 71.4 | 57.3 | 55.4 | 52.7 | 49.5 | −14.1 | −16.0 | −18.7 | −21.9 |
| 1312- | TATTGCACAATGGGCGCAAGCC | | | | | | | | | |
| 35_004 | TG | | | | | | | | | |
| rrs_ | ATTO488- | 70.3 | 56.9 | 55.4 | 52.8 | 50.4 | −13.4 | −14.9 | −17.5 | −19.9 |
| 1312- | TATTGCACAATGGGCGCAAGCC | | | | | | | | | |
| 33_005 | | | | | | | | | | |

TABLE 7

| Effect of two nucleobase | | | | | | | | | | |
|---|---|---|---|---|---|---|---|---|---|---|
| mismatches or abasic sites | | | | | | | | | | |
| in probe on Tm. LightCycler | | | | | | | | | | |
| 2.0 determination of Tm | | | | | | | | | | |
| (° C.) in 50 mM phosphate | | | | | | | | | | |
| buffer with 100 mM NaCl and | | | | | | | | | | |
| 0.1 mM EDTA at pH 7.0. The | | | rrs_ | rrs_ | rrs_ | | rrs_ | rrs_ | rrs_ | |
| nucleobases for which the | | rrs_ | 1341- | 1341- | 1341- | | 1341- | 1341- | 1341- | |
| complementary nucleobases | | 1341- | 12_069 | 12_076 | 12_083 | | 12_069 | 12_076 | 12_083 | |
| are altered are highlighted | | 12_049 | A | T | C | rrs_ | A | T | C | rrs_ |
| in underlined bold. The | | G | nucleo- | nucleo- | nucleo- | 1341- | nucleo- | nucleo- | nucleo- | 1341- |
| change in Tm towards Tm for | | nucleo- | tide | tide | tide | 12_090 | tide | tide | tide | 12_090 |
| the match base pairs (ΔTm) | | tide | mis- | mis- | mis- | Abasic | mis- | mis- | mis- | Abasic |
| are shown to the right. | | matches | matches | matches | matches | sites | matches | matches | matches | sites |
| rrs_ | ATTO488- | 76.1 | 61.9 | 61.8 | 59.6 | 52.5 | −14.2 | −14.3 | −16.5 | −23.6 |
| 1312- | TATTGCACAATGGGCGCAAGCC | | | | | | | | | |
| 41_001 | TGATGCAG | | | | | | | | | |
| rrs_ | ATTO488- | 75.4 | 60.3 | 60.2 | 58.1 | 51.0 | −15.1 | −15.2 | −17.3 | −24.4 |
| 1312- | TATTGCACAATGGGCGCAAGCC | | | | | | | | | |
| 39_002 | TGATGC | | | | | | | | | |
| rrs_ | ATTO488- | 72.5 | 54.7 | 55.1 | 53.1 | 47.6 | −17.8 | −17.4 | −19.4 | −24.9 |
| 1312- | TATTGCACAATGGGCGCAAGCC | | | | | | | | | |
| 37_003 | TGAT | | | | | | | | | |
| rrs_ | ATTO488- | 71.4 | 52.3 | 53.2 | 51.3 | 47.5 | −19.1 | −18.2 | −20.1 | −23.9 |
| 1312- | TATTGCACAATGGGCGCAAGCC | | | | | | | | | |
| 35_004 | TG | | | | | | | | | |
| rrs_ | ATTO488- | 70.3 | 51.4 | 53.3 | 52.3 | 48.5 | −18.9 | −17.0 | −18.0 | −21.8 |
| 1312- | TATTGCACAATGGGCGCAAGCC | | | | | | | | | |
| 33_005 | | | | | | | | | | |

Table 6 and Table 7. Effect on Tm by two nucleobase mismatches or two abasic sites determined for oligonucleotides with lengths from 22 to 30 nucleotides.

For a 30-mer duplex probe with three nucleobase mismatches, the average Tm decreased by 21.5° C. (range 19.3° C. to 24.3° C.) compared to Tm for matching oligonucleotide probes. The average decrease in Tm by three abasic sites was 31.4° C. or an additional decrease of 46% for three abasic sites compared to three nucleobase mismatches (Table 8). Likewise for a 22-mer duplex probe, the average Tm decrease by three nucleobase mismatches was above 26.3° C. (range from 25.0° C.) compared to above 30.3° for three abasic sites.

TABLE 8

| Effect of three nucleobase | | | | | | | | | | |
|---|---|---|---|---|---|---|---|---|---|---|
| mismatches or abasic sites | | | | | | | | | | |
| in probe on Tm. LightCycler | | | | | | | | | | |
| 2.0 determination of Tm | | | | | | | | | | |
| (° C.) in 50 mM phosphate | | | | | | | | | | |
| buffer with 100 mM NaCl and | | | | | | | | | | |
| 0.1 mM EDTA at pH 7.0. The | | | rrs_ | rrs_ | rrs_ | | rrs_ | rrs_ | rrs_ | |
| nucleobases for which the | | rrs_ | 1341- | 1341- | 1341- | | 1341- | 1341- | 1341- | |
| complementary nucleobases | | 1341- | 12_070 | 12_077 | 12_084 | | 12_070 | 12_077 | 12_084 | |
| are altered are highlighted | | 12_049 | A | T | C | rrs_ | A | T | C | rrs_ |
| in underlined bold. The | | G | nucleo- | nucleo- | nucleo- | 1341- | nucleo- | nucleo- | nucleo- | 1341- |
| change in Tm towards Tm for | | nucleo- | tide | tide | tide | 12_091 | tide | tide | tide | 12_091 |
| the match base pairs (ΔTm) | | tide | mis- | mis- | mis- | Abasic | mis- | mis- | mis- | Abasic |
| are shown to the right. | | matches | matches | matches | matches | sites | matches | matches | matches | sites |
| rrs_ | ATTO488- | 76.1 | 57.5 | 54.6 | 51.8 | 44.7 | −18.6 | −21.5 | −24.3 | −31.4 |
| 1312- | TATTGCACAATGGGCGCAAGCC | | | | | | | | | |
| 41_001 | TGATGCAG | | | | | | | | | |
| rrs_ | ATTO488- | 75.4 | 55.5 | 52.4 | 48.7 | 43.0 | −19.9 | −23.0 | −26.7 | −32.4 |
| 1312- | TATTGCACAATGGGCGCAAGCC | | | | | | | | | |
| 39_002 | TGATGC | | | | | | | | | |
| rrs_ | ATTO488- | 72.5 | 49.8 | 46.5 | 42.3 | <40 | −22.7 | −26.0 | −30.2 | >−32.5 |
| 1312- | TATTGCACAATGGGCGCAAGCC | | | | | | | | | |
| 37_003 | TGAT | | | | | | | | | |
| rrs_ | ATTO488- | 71.4 | 47.0 | 43.4 | 40.2 | <40 | −24.4 | −28.0 | −31.2 | >−31.4 |
| 1312- | TATTGCACAATGGGCGCAAGCC | | | | | | | | | |
| 35_004 | TG | | | | | | | | | |
| rrs_ | ATTO488- | 70.3 | 45.3 | 42.8 | <40 | <40 | −25.0 | −27.5 | >−30.3 | >−30.3 |
| 1312- | TATTGCACAATGGGCGCAAGCC | | | | | | | | | |
| 33_005 | | | | | | | | | | |

Table 8. Effect on Tm by three nucleobase mismatches or three abasic sites determined for oligonucleotides with lengths from 22 to 30 nucleotides.

Table 9 to 13 show the effect on Tm of combination of one to three abasic site(s) in the oligonucleotide target sequence in hybridization with complementary oligonucleotide probes with A, T, C or G nucleotide(s), abasic site(s) or o-TINA molecule(s) positioned complementary to the abasic site(s).

For a 30-mer duplex target with an abasic site placed opposite to one of the natural nucleobases, the Tm compared to the Tm for the match base pair was decreased in average by 5.3° C. (range 3.7° C. to 7.2° C.). When both strands in the duplex had an abasic site, the Tm decreased in average with 8.8° C. (range 7.3° C. to 10.2° C.) or an additional decrease of 66% compared to an abasic site opposite a nucleobase. By placing an ortho-TINA molecule in the probe opposite to the abasic site in the target, the Tm compared to the Tm for oligonucleotides with match base pair was decreased in average by 1.6° C. (1.5° C. to 1.6° C.). For a 22-mer duplex target, the average Tm decrease in Tm for an abasic site in the target placed opposite a nucleobase in the probe was in average 5.8° C. (range 2.3° C. to 9.2° C.). When an abasic site in the target was placed opposite an abasic site in the probe, the decrease in Tm was in average 9.4° C. (range 5.8° C. to 12.9° C. or an additional decrease of 62% compared to an abasic site opposite a nucleobase. Placement of an ortho-TINA molecule in the probe opposite the abasic site in a 22-mer duplex target decreased the Tm in average by 1.6° C. (range 3.6° C. to an increase in Tm by 0.4° C.) compared to the Tm for a duplex with match base pair. (data are presented in Table 9 and 10).

| One abasic site in the 5′ | | | | | | | |
|---|---|---|---|---|---|---|---|
| part of the target oligo- | | | | | | | |
| nucleotides. LightCycler | | | | | | | |
| 2.0 determination of Tm | | | | | | | |
| (° C.) in 50 mM phosphate | rrs_ | | | | | | |
| buffer with 100 mM NaCl | 1341- | | | | | | |
| and 0.1 mM EDTA at pH 7.0. | 12_049 | | | | | | |
| The abasic site (B) is | Match | rrs_ | rrs_ | rrs_ | rrs_ | | rrs_ |
| highlighted in underlined | Tm for | 1341- | 1341- | 1341- | 1341- | rrs_ | 1341- |
| bold. The change in Tm | 049 | 12_049 | 12_064 | 12_071 | 12_078 | 1341- | 12_050 |
| compared to Tm for the | toward | G | A | T | C | 12_085 | o-TINA |
| match base pairs (ΔTm) | 001 to | nucleo- | nucleo- | nucleo- | nucleo- | Abasic | substi- |
| are shown to the right. | 007 | tide | tide | tide | tide | site | tution |
| Table 9a | | | | | | | |
| ATTO488- | 76.1 | 72.4 | 72.1 | 72.1 | 72.3 | 68.8 | 74.5 |
| TATTGBACAATGGGCGCAAGCCTGATGCAG | | | | | | | |
| ATTO488- | 75.4 | 70.8 | 70.7 | 70.5 | 71.1 | 67.7 | 74.0 |
| TATTGBACAATGGGCGCAAGCCTGATGC | | | | | | | |
| ATTO488- | 72.5 | 66.0 | 66.5 | 65.7 | 67.1 | 63.4 | 69.7 |
| TATTGBACAATGGGCGCAAGCCTGAT | | | | | | | |
| ATTO488- | 71.4 | 64.3 | 63.7 | 64.1 | 65.2 | 61.8 | 68.4 |
| TATTGBACAATGGGCGCAAGCCTG | | | | | | | |
| ATTO488- | 70.3 | 61.4 | 61.1 | 61.2 | 62.5 | 57.4 | 66.7 |
| TATTGBACAATGGGCGCAAGCC | | | | | | | |
| ATTO488- | 68.6 | 58.0 | 58.3 | 58.3 | 59.3 | 55.9 | 63.5 |
| TATTGBACAATGGGCGCAAG | | | | | | | |
| ATTO488- | 66.8 | 54.8 | 55.4 | 54.2 | 55.5 | 53.2 | 61.5 |
| TATTGBACAATGGGCGCA | | | | | | | |
| Table 9b | | | | | | | |
| ATTO488- | | −3.7 | −4.0 | −4.0 | −3.8 | −7.3 | −1.6 |
| TATTGBACAATGGGCGCAAGCCTGATGCAG | | | | | | | |
| ATTO488- | | −4.6 | −4.7 | −4.9 | −4.3 | −7.7 | −1.4 |
| TATTGBACAATGGGCGCAAGCCTGATGC | | | | | | | |
| ATTO488- | | −6.5 | −6.0 | −6.8 | −5.4 | −9.1 | −2.8 |
| TATTGBACAATGGGCGCAAGCCTGAT | | | | | | | |
| ATTO488- | | −7.1 | −7.7 | −7.3 | −6.2 | −9.6 | −3.0 |
| TATTGBACAATGGGCGCAAGCCTG | | | | | | | |
| ATTO488- | | −8.9 | −9.2 | −9.1 | −7.8 | −12.9 | −3.6 |
| TATTGBACAATGGGCGCAAGCC | | | | | | | |
| ATTO488- | | −10.6 | −10.3 | −10.3 | −9.3 | −12.7 | −5.1 |
| TATTGBACAATGGGCGCAAG | | | | | | | |
| ATTO488- | | −12.0 | −11.4 | −12.6 | −11.3 | −13.6 | −5.3 |
| TATTGBACAATGGGCGCA | | | | | | | |

| One abasic site in the 3′ | | | | | | | |
|---|---|---|---|---|---|---|---|
| part of the target oligo- | | | | | | | |
| nucleotides. LightCycler | | | | | | | |
| 2.0 determination of Tm | | | | | | | |
| (° C.) in 50 mM phosphate | rrs_ | | | | | | |
| buffer with 100 mM NaCl | 1341- | | | | | | |
| and 0.1 mM EDTA at pH 7.0. | 12_049 | | | | | | |
| The abasic site (B) is | Match | rrs_ | rrs_ | rrs_ | rrs_ | | rrs_ |
| highlighted in underlined | Tm for | 1341- | 1341- | 1341- | 1341- | rrs_ | 1341- |
| bold. The change in Tm | 049 | 12_049 | 12_066 | 12_073 | 12_080 | 1341- | 12_052 |
| compared to Tm for the | toward | G | A | T | C | 12_087 | o-TINA |
| match base pairs (ΔTm) | 001 to | nucleo- | nucleo- | nucleo- | nucleo- | Abasic | substi- |
| are shown to the right. | 007 | tide | tide | tide | tide | site | tution |
| Table 10a | | | | | | | |
| ATTO488- | 76.1 | 69.8 | 68.9 | 69.3 | 69.2 | 65.9 | 74.6 |
| TATTGCACAATGGGCGCAAGBCTGATGCAG | | | | | | | |
| ATTO488- | 75.4 | 68.9 | 67.7 | 67.8 | 68.0 | 64.8 | 73.3 |
| TATTGCACAATGGGCGCAAGBCTGATGC | | | | | | | |
| ATTO488- | 72.5 | 67.0 | 66.5 | 67.2 | 66.3 | 64.1 | 71.0 |
| TATTGCACAATGGGCGCAAGBCTGAT | | | | | | | |
| ATTO488- | 71.4 | 67.4 | 66.7 | 67.4 | 67.1 | 63.7 | 71.0 |
| TATTGCACAATGGGCGCAAGBCTG | | | | | | | |
| ATTO488- | 70.3 | 68.0 | 67.0 | 67.6 | 67.2 | 64.5 | 70.7 |
| TATTGCACAATGGGCGCAAGBC | | | | | | | |
| Table 10b | | | | | | | |
| ATTO488- | | −6.3 | −7.2 | −6.8 | −6.9 | −10.2 | −1.5 |
| TATTGCACAATGGGCGCAAGBCTGATGCAG | | | | | | | |
| ATTO488- | | −6.5 | −7.7 | −7.6 | −7.4 | −10.6 | −2.1 |
| TATTGCACAATGGGCGCAAGBCTGATGC | | | | | | | |
| ATTO488- | | −5.5 | −6.0 | −5.3 | −6.2 | −8.4 | −1.5 |
| TATTGCACAATGGGCGCAAGBCTGAT | | | | | | | |
| ATTO488- | | −4.0 | −4.7 | −4.0 | −4.3 | −7.7 | −0.4 |
| TATTGCACAATGGGCGCAAGBCTG | | | | | | | |
| ATTO488- | | −2.3 | −3.3 | −2.7 | −3.1 | −5.8 | 0.4 |
| TATTGCACAATGGGCGCAAGBC | | | | | | | |

Table 9 (Table 9=table 9a+table 9b) and table 10 (table 10=table 10a+table 10b). Effect on Tm by a single abasic site in the target placed opposite a nucleobase mismatch, an other abasic site or an ortho-TINA molecule in the probe.

For a 30-mer duplex target with two abasic sites placed opposite two nucleobases in the probe, the Tm compared to the Tm for the duplex with match base pairs was decreased in average by 13.5° C. (range 10.8° C. to 17.2° C.). When both strands in the duplex had two abasic sites, the Tm decreased in average with 17.7° C. (range 15.4° C. to 20.6° C.) or an additional decrease of 31% compared with two abasic sites opposite nucleobases. By placing two ortho-TINA molecules in the probe opposite the abasic sites in the target, the Tm compared to the Tm for oligonucleotides with match base pairs was decreased in average by 4.5° C. (3.3° C. to 6.4° C.). For a 22-mer duplex target, the average Tm decrease in Tm for two abasic sites placed opposite nucleobases in the probe was in average 15.9° C. (range 11.2° C. to 21.5° C.). When two abasic sites in the probe was placed opposite abasic sites in the target the decrease in Tm was in average 19.6° C. (range 16.9° C. to 23.2° C. or an additional decrease of 23% compared with two abasic sites opposite nucleobases. Placement of two ortho-TINA molecules in the probe opposite the abasic sites in a 22-mer duplex target decreased the Tm in average by 5.8° C. (range 3.3° C. to 7.2° C.) compared to the Tm for a duplex with match base pair. (data are presented in Table 11 and 12).

| Two abasic sites in the 5′ | | | | | | | |
|---|---|---|---|---|---|---|---|
| and center part of the | | | | | | | |
| target oligonucleotides. | | | | | | | |
| LightCycler 2.0 determina- | | | | | | | |
| tion of Tm (° C.) in 50 mM | rrs_ | | | | | | |
| phosphate buffer with 100 | 1341- | | | | | | |
| mM NaCl and 0.1 mM EDTA at | 12_049 | | | | | | |
| pH 7.0. The abasic sites | Match | rrs_ | rrs_ | rrs_ | rrs_ | | rrs_ |
| (B) are highlighted in | Tm for | 1341- | 1341- | 1341- | 1341- | rrs_ | 1341- |
| underlined bold. The change | 049 | 12_049 | 12_067 | 12_074 | 12_081 | 1341- | 12_053 |
| in Tm compared to Tm for the | toward | G | A | T | C | 12_088 | o-TINA |
| match base pairs (ΔTm) are | 001 to | nucleo- | nucleo- | nucleo- | nucleo- | Abasic | substi- |
| shown to the right. | 007 | tide | tide | tide | tide | site | tution |
| Table 11a | | | | | | | |
| ATTO488- | 76.1 | 64.8 | 65.3 | 62.5 | 64.1 | 58.9 | 72.3 |
| TATTGBACAATGGGBGCAAGCCTGATGCAG | | | | | | | |
| ATTO488- | 75.4 | 62.7 | 63.1 | 60.6 | 62.0 | 56.8 | 70.3 |
| TATTGBACAATGGGBGCAAGCCTGATGC | | | | | | | |
| ATTO488- | 72.5 | 57.1 | 58.3 | 55.1 | 56.4 | 51.8 | 67.1 |
| TATTGBACAATGGGBGCAAGCCTGAT | | | | | | | |
| ATTO488- | 71.4 | 54.7 | 56.2 | 52.7 | 54.2 | 50.2 | 66.2 |
| TATTGBACAATGGGBGCAAGCCTG | | | | | | | |
| ATTO488- | 70.3 | 50.5 | 52.6 | 48.8 | 49.8 | 47.0 | 63.4 |
| TATTGBACAATGGGBGCAAGCC | | | | | | | |
| ATTO488- | 68.6 | 42.3 | 44.7 | 41.5 | 41.6 | <40 | 56.9 |
| TATTGBACAATGGGBGCAAG | | | | | | | |
| ATTO488- | 66.8 | <40 | <40 | <40 | <40 | <40 | 53.7 |
| TATTGBACAATGGGBGCA | | | | | | | |
| Table 11b | | | | | | | |
| ATTO488- | | −11.3 | −10.8 | −13.6 | −12.0 | −17.2 | −3.8 |
| TATTGBACAATGGGBGCAAGCCTGATGCAG | | | | | | | |
| ATTO488- | | −12.7 | −12.3 | −14.8 | −13.4 | −18.6 | −5.1 |
| TATTGBACAATGGGBGCAAGCCTGATGC | | | | | | | |
| ATTO488- | | −15.4 | −14.2 | −17.4 | −16.1 | −20.7 | −5.4 |
| TATTGBACAATGGGBGCAAGCCTGAT | | | | | | | |
| ATTO488- | | −16.7 | −15.2 | −18.7 | −17.2 | −21.2 | −5.2 |
| TATTGBACAATGGGBGCAAGCCTG | | | | | | | |
| ATTO488- | | −19.8 | −17.7 | −21.5 | −20.5 | −23.3 | −6.9 |
| TATTGBACAATGGGBGCAAGCC | | | | | | | |
| ATTO488- | | −26.3 | −23.9 | −27.1 | −27.0 | >−28.6 | −11.7 |
| TATTGBACAATGGGBGCAAG | | | | | | | |
| ATTO488- | | >−26.8 | >−26.8 | >−26.8 | >−26.8 | >−26.8 | −13.1 |
| TATTGBACAATGGGBGCA | | | | | | | |

TABLE 12

| Two abasic sites in the 5′ | | | | | | | |
|---|---|---|---|---|---|---|---|
| and 3′ part of the target | | | | | | | |
| oligonucleotides. LightCycler | | | | | | | |
| 2.0 determination of Tm | | | | | | | |
| (° C.) in 50 mM phosphate | rrs_ | | | | | | |
| buffer with 100 mM NaCl and | 1341- | | | | | | |
| 0.1 mM EDTA at pH 7.0. The | 12_049 | | | | | | |
| abasic sites (B) are high- | Match | rrs_ | rrs_ | rrs_ | rrs_ | | rrs_ |
| lighted in underlined bold. | Tm for | 1341- | 1341- | 1341- | 1341- | rrs_ | 1341- |
| The change in Tm compared | 049 | 12_049 | 12_068 | 12_075 | 12_082 | 1341- | 12_054 |
| to Tm for the match base | toward | G | A | T | C | 12_089 | o-TINA |
| pairs (ΔTm) are shown to | 001 to | nucleo- | nucleo- | nucleo- | nucleo- | Abasic | substi- |
| the right. | 007 | tide | tide | tide | tide | site | tution |
| ATTO488- | 76.1 | 64.3 | 64.4 | 63.3 | 63.4 | 60.7 | 72.8 |
| TATTGBACAATGGGCGCAAGBCTGATGCAG | | | | | | | |
| ATTO488- | 75.4 | 62.8 | 63.0 | 61.8 | 61.7 | 58.8 | 71.4 |
| TATTGBACAATGGGCGCAAGBCTGATGC | | | | | | | |
| ATTO488- | 72.5 | 57.6 | 59.2 | 57.6 | 57.1 | 54.8 | 68.0 |
| TATTGBACAATGGGCGCAAGBCTGAT | | | | | | | |
| ATTO488- | 71.4 | 57.7 | 58.8 | 57.4 | 56.8 | 53.0 | 67.8 |
| TATTGBACAATGGGCGCAAGBCTG | | | | | | | |
| ATTO488- | 70.3 | 57.7 | 59.1 | 58.0 | 57.3 | 53.4 | 67.0 |
| TATTGBACAATGGGCGCAAGBC | | | | | | | |
| ATTO488- | | −11.8 | −11.7 | −12.8 | −12.7 | −15.4 | −3.3 |
| TATTGBACAATGGGCGCAAGBCTGATGCAG | | | | | | | |
| ATTO488- | | −12.6 | −12.4 | −13.6 | −13.7 | −16.6 | −4.0 |
| TATTGBACAATGGGCGCAAGBCTGATGC | | | | | | | |
| ATTO488- | | −14.9 | −13.3 | −14.9 | −15.4 | −17.7 | −4.5 |
| TATTGBACAATGGGCGCAAGBCTGAT | | | | | | | |
| ATTO488- | | −13.7 | −12.6 | −14.0 | −14.6 | −18.4 | −3.6 |
| TATTGBACAATGGGCGCAAGBCTG | | | | | | | |
| ATTO488- | | −12.6 | −11.2 | −12.3 | −13.0 | −16.9 | −3.3 |
| TATTGBACAATGGGCGCAAGBC | | | | | | | |

Table 11 (Table 11=table 11a+table 11b) and table 12 (table 12=table 12a+table 12b). Effect on Tm by two abasic sites in the target placed opposite nucleobase mismatches, other abasic sites or ortho-TINA molecules in the probe.

For a 30-mer duplex target with three abasic sites placed opposite three nucleobases in the probe, the Tm compared to the Tm for the duplex with match base pairs was decreased in average by 22.7° C. (range 21.4° C. to 24.3° C.). When both strands in the duplex had three abasic sites, the Tm decreased with 27.4° C. or an additional decrease of 21% compared with three abasic sites opposite nucleobases. By placing three ortho-TINA molecules in the probe opposite the abasic sites in the target, the Tm compared to the Tm for oligonucleotides with match base pairs was decreased by 8.5° C. For a 22-mer duplex target, the average Tm decrease in Tm for three abasic sites placed opposite nucleobases in the probe was in average 27.9° C. (range 26.2° C. to 29.2° C.). When three abasic sites in the probe was placed opposite abasic sites in the target, the decrease in Tm was above 30.3° C. (the exact determination was limited by the LightCycler 2.0). Placement of three ortho-TINA molecules in the probe opposite the abasic sites in a 22-mer duplex target decreased the Tm by 11.8° C. compared with the Tm for a duplex with match base pair. (data are presented in Table 13).

Table 13. Effect on Tm by three abasic sites in the target placed opposite nucleobase mismatches, other abasic sites or ortho-TINA molecules in the probe.

TABLE 13

| Three abasic sites in the 5′, | | | | | | | |
|---|---|---|---|---|---|---|---|
| center and 3′ part of the tar- | | | | | | | |
| get oligonucleotides. Light- | | | | | | | |
| Cycler 2.0 determination of | rrs_ | | | | | | |
| Tm (° C.) in 50 mM phosphate | 1341- | | | | | | |
| buffer with 100 mM NaCl and | 12_049 | | | | | | |
| 0.1 mM EDTA at pH 7.0. The | Match | rrs_ | rrs_ | rrs_ | rrs_ | | rrs_ |
| abasic sites (B) are high- | Tm for | 1341- | 1341- | 1341- | 1341- | rrs_ | 1341- |
| lighted in underlined bold. | 049 | 12_049 | 12_070 | 12_077 | 12_084 | 1341- | 12_056 |
| The change in Tm compared to | toward | G | A | T | C | 12_091 | o-TINA |
| Tm for the match base pairs | 001 to | nucleo- | nucleo- | nucleo- | nucleo- | Abasic | substi- |
| (ΔTm) are shown to the right. | 007 | tide | tide | tide | tide | site | tution |
| ATTO488- | 76.1 | 54.4 | 54.7 | 51.8 | 52.8 | 48.7 | 67.6 |
| TATTGBACAATGGGBGCAAGBCTGATGCAG | | | | | | | |
| ATTO488- | 75.4 | 51.7 | 52.6 | 50.6 | 50.1 | 46.7 | 66.2 |
| TATTGBACAATGGGBGCAAGBCTGATGC | | | | | | | |
| ATTO488- | 72.5 | 44.8 | 46.2 | 42.7 | 43.6 | <40 | 60.9 |
| TATTGBACAATGGGBGCAAGBCTGAT | | | | | | | |
| ATTO488- | 71.4 | 42.8 | 44.1 | 41.1 | 41.7 | <40 | 60.0 |
| TATTGBACAATGGGBGCAAGBCTG | | | | | | | |
| ATTO488- | 70.3 | 42.7 | 44.1 | 41.1 | 41.6 | <40 | 58.5 |
| TATTGBACAATGGGBGCAAGBC | | | | | | | |
| ATTO488- | | −21.7 | −21.4 | −24.3 | −23.3 | −27.4 | −8.5 |
| TATTGBACAATGGGBGCAAGBCTGATGCAG | | | | | | | |
| ATTO488- | | −23.7 | −22.8 | −24.8 | −25.3 | −28.7 | −9.2 |
| TATTGBACAATGGGBGCAAGBCTGATGC | | | | | | | |
| ATTO488- | | −27.7 | −26.3 | −29.8 | −28.9 | >−32.5 | −11.6 |
| TATTGBACAATGGGBGCAAGBCTGAT | | | | | | | |
| ATTO488- | | −28.6 | −27.3 | −30.3 | −29.7 | >−31.4 | −11.4 |
| TATTGBACAATGGGBGCAAGBCTG | | | | | | | |
| ATTO488- | | −27.6 | −26.2 | −29.2 | −28.7 | >−30.3 | −11.8 |
| TATTGBACAATGGGBGCAAGBC | | | | | | | |

### REFERENCES

[1] J. Natl. Cancer Inst 2009 101 (18) 1244 Nagasaka T.
[2] Future Oncol. 2010 6 (3) 333 Goel.
[3] Bioorganic & Medicinal Chemistry 16 (2008) 9937-9947 Osman et al.
[4] Chem. Eur. J. 2008, D01:10.1002/chem200800380 1-, 2-, and 4-ethynylpyrenes in the structure of twisted intercalating nucleic acids: structure, thermal stability, and fluorescence relationship. Filichev et al.
[5] Bioconjugate Chem. 2006, 17, 950-957. Geci et al.
[6] J. Am. Chem. Soc. 1998, 120, 6191-6192. Matray and Kool.
[7] Nucleic acids research, vol. 15, 19, 1987 pp. 7823-7830
[8] Nucleic acids Research, vol. 18, No. 13 1990, pp. 3841-3845.
[9] EMBO Journal vol. 15 no. 13 pp. 3442-3447, 1996. Kavli et al.

1. A method for capturing a target polynucleotide from a sample obtained from biological material, the method comprising the steps of:
(i) providing a double stranded target polynucleotide;
(ii) destabilizing said double stranded target polynucleotide by removing one or more bases, such as one or more of the nucleobases A, T, U, C or G, 5-hydroxymethyl-dC, 5-methylcytosine (m⁵C), pseudouridine (Ψ), dihydrouridine (D), inosine (I), 7-methylguanosine (m⁷G), hypoxanthine, xanthine and their 2′-O-Methyl-derivatives and/or N-Methyl-derivatives from said target polynucleotide, thereby generating one or more abasic sites and
(iii) denaturing said destabilized double stranded target polynucleotide to generate single stranded target polynucleotide, and
(iv) capturing said single stranded target polynucleotide with a complementary oligonucleotide probe having a length of 15 to 35 nucleotides, wherein the complementary oligonucleotide probe comprises one or more intercalator molecules inserted into the backbone-structure of said oligonucleotide probe, wherein said one or more intercalator molecule(s) fit morphologically into the one or more abasic sites of the complementary polynucleotide target sequence.

2. The method according to preceding claim 1 wherein the complementary oligonucleotide probe comprises naturally occurring nucleotides and/or nucleotides which are not known to occur in nature such as those selected from the group consisting of RNA, α-L-RNA, β-D-RNA, 2′-R-RNA, DNA, LNA, PNA, PMO, TNA, GNA, oligonucleotide N3′→P5′ phosphoramidates, BNA, α-L-LNA, HNA, MNA, ANA, CAN, INA, CeNA, (2′-NH)-TNA, (3′—NH)-TNA, α-L-Ribo-LNA, α-L-Xylo-LNA, β-D-Ribo-LNA, β-D-Xylo-LNA, [3.2.1]-LNA, Bicyclo-DNA, 6-Amino-Bicyclo-DNA, 5-epi-Bicyclo-DNA, α-Bicyclo-DNA, Tricyclo-DNA, Bicyclo[4.3.0]-DNA, Bicyclo[3.2.1]-DNA, Bicyclo[4.3.0]amide-DNA, β-D-Ribopyranosyl-NA, α-L-Lyxopyranosyl-NA, 2′-OR-RNA, 2′-AE-RNA, and combinations and modifications thereof.

3. The method according to claim 1, wherein the method further comprises one or more washing steps in order to remove unbound nucleotide material.

4. The method according to claim 1, wherein the one or more abasic sites are 2 or more abasic sites, such as 3 or more abasic sites.

5. The method according to claim 1, wherein the method further comprises conversion of one or more types of bases in the double stranded target polynucleotide to another chemical entity.

6. The method according to claim 5, wherein the method further comprises destabilisation of said double stranded target polynucleotide by removal of one or more chemical entities from said double stranded target polynucleotide.

7. The method according to claim 1, wherein the method further comprises conversion of one or more C's in the target polynucleotide to one or more U's.

8. The method according to claim 7, wherein the conversion of one or more C's in the target polynucleotide to one or more U's is preformed by bisulphite treatment.

9. The method according to claim 1, wherein A is removed from said double stranded target polynucleotide and/or single stranded polynucleotide.

10. The method according to claim 1, wherein T is removed from said double stranded target polynucleotide and/or single stranded polynucleotide.

11. The method according to claim 1, wherein C is removed from said double stranded target polynucleotide and/or single stranded Polynucleotide.

12. The method according to claim 1, wherein G is removed from said double stranded target polynucleotide and/or single stranded Polynucleotide.

13. The method according to claim 1, wherein U is removed from said double stranded target Polynucleotide and/or single stranded Polynucleotide.

14. The method according to claim 1, wherein the removal is performed by one or more enzymes and/or physical stress and/or temperature change.

15. The method according to claim 13, wherein the removal of U is performed by use of uracil dehydrogenase.

16. The method according to claim 9, wherein the removal of A is performed by adjustment of the pH.

17. The method according to claim 1, wherein 1, 2, or 3 types of the bases from the target polynucleotide is/are removed.

18. The method according to claim 1, wherein the total number of bases that are removed from the target Polynucleotide can be selected from the group consisting of 1, 2, 3, 4, 5, 6, 7, 8, 9, 10, 11, 12, 13, 14, 15, 16, 17, 18, 19, 20 and more than 20 bases.

19. The method according to claim 1, wherein the total number of intercalator molecules can be selected from the group consisting of 1, 2, 3, 4, 5, 6, 7, 8, 9, 10, 11, 12, 13, 14, 15, 16, 17, 18, 19, 20 and more than 20 intercalator molecules.

20. The method according to claim 1, wherein an intercalator molecule has been inserted into from 10% to 100% of the abasic sites in the target DNA and/or RNA such as from 10% to 20%, for example from 20% to 30%, such as from 30% to 40%, for example from 40% to 50%, such as from 50% to 60%, for example from 60% to 70%, such as from 70% to 80%, for example from 80% to 90%, such as from 90% to 100%, or any combination thereof.

21. The method according to claim 1, wherein an intercalator molecule has been inserted into more than 10% of the abasic sites in the target DNA and/or RNA, such as more than 20%, for example more than 30%, such as more than 40%, for example more than 50%, such as more than 60%, for example more than 70%, such as more than 80%, for example more than 90%, such as more than 95%, for example 100%.

22. The method according to claim 1, wherein the insertion of the intercalator molecules results in increased melting temperature of the polynucleotide duplex consisting of the target Polynucleotide and the complementary probe.

23. The method according to claim 1, wherein the ratio between the number of intercalator molecules and the total number of bases in the complementary probe is from 1:50 to 1:2 such as from 1:50 to 1:40, for example 1:40 to 1:30, such as from 1:30 to 1:20, for example 1:20 to 1:10, such as from 1:10 to 1:5, for example 1:5 to 1:2, or any combination of these intervals.

24. The method according to claim 1, wherein the one or more intercalator molecules can be selected from the group consisting of TINA, INA, ortho-TINA, para-TINA, and AMANY.

25. The method according claim 1, wherein the size of the intercalator molecule is between 20 and 400 Å, such as from 20-40 Å, for example from 40-60 Å, such as from 60-80 Å, for example from 80-100 Å, such as from 100-120 Å, for example from 120-140 Å, such as from 140-160 Å, for example from 160-180 Å, such as from 180-200 Å, for example from 200-220 Å, such as from 220-240 Å, for example from 240-260 Å, such as from 260-280 Å, for example from 280-300 Å, such as from 300-320 Å, for example from 320-340 Å, such as from 340-360 Å, for example from 360-380 Å, such as from 380-400 Å, or any combination of these intervals.

26. The method according to claim 1, wherein the complementary probe comprises more than one type of intercalator molecules such as 2, 3, 4, 5 or more than 5 different types of intercalator molecules.

27. The method according to claim 1, wherein the complementary probe is connected to a support.

28. The method according to claim 27, wherein the support is selected from the group consisting of Poly(ether ether ketone) (PEEK), PP (polypropylene), PE (polyethylene), Poly(ethylene terephthalate) (PET), Poly(vinyl chloride) (PVC), Polyamide/nylon (PA), Polycarbonate (PC), Cyclic olefin copolymer (COC), Filter paper, Cotton, Cellulose, Poly(4-vinylbenzyl chloride) (PVBC), Poly(vinylidene fluoride) (PVDF), Polystyrene (PS), Toyopearl®, Hydrogels, Polyimide (PI), 1,2-Polybutadiene (PB), LSR (Liquid silicon rubber), poly(dimethylsiloxane) (PDMS), fluoropolymers-and copolymers (e.g. poly(tetrafluoroethylene) (PTFE), Perfluoroethylene propylene copolymer (FEP), Ethylene tetrafluoroethylene (ETFE)), poly(methyl methacrylate) (PMMA), Nanoporous materials, Membranes, Mesostructured cellular foam (MCF), and singlewall or multiwall Carbon Nanotubes (SWCNT, MWCNT), particulate matters, beads, magnetic beads, non-magnetic beads, polystyrene beads, magnetic polystyrene beads, sepharose beads, sephacryl beads, polystyrene beads, agarose beads, polysaccharide beads, and polycarbamate beads.

29. The method according to claim 27, wherein the support is a solid support.

30. The method according to claim 29, wherein the solid support is selected from the group consisting of microtiter plate or other plate formats, reagent tubes, glass slides or other supports for use in array or microarray analysis, tubings or channels of micro fluidic chambers or devices and Biacore chips.

31. (canceled)

32. The method according to claim 1, wherein the complementary probe comprises one or more labels.

33. The method according to claim 32, wherein the one or more labels is selected from the group consisting of biotin, a fluorescent label, 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, dyes, Cy2, Cy3, and Cy5, PerCP, phycobiliproteins, R-phycoerythrin (RPE), allophycoerythrin (APC), Texas Red, Princeston Red, Green fluorescent protein (GFP) and analogues thereof, conjugates of R-phycoerythrin or allophycoerythrin, inorganic fluorescent labels based on semiconductor nanocrystals (like quantum dot and Qdot™ nanocrystals), time-resolved fluorescent labels based on lanthanides like Eu3+ and Sm3+, haptens, DNP, digoxiginin, enzymic labels, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase and glucose oxidase (GO), luminiscence labels, luminol, isoluminol, acridinium esters, 1,2-dioxetanes, pyridopyridazines, radioactivity labels, isotopes of iodide, isotopes of cobalt, isotopes of elenium, isotopes of tritium, and isotopes of phosphor.

34. The method according to claim 33, wherein the biotin is detected by use of streptavidin-R-phycoerythrine.

35. The method according to claim 32, wherein the method further comprises a washing step prior to and/or after addition of the detection probe.

36. The method according to claim 32, wherein complementary detection probe comprises one or more intercalator molecules.

37. The method according to claim 36, wherein the total number of intercalator molecules can be selected from the group consisting of 1, 2, 3, 4, 5, 6, 7, 8, 9, 10, 11, 12, 13, 14, 15, 16, 17, 18, 19, 20 and more than 20 different or identical intercalator molecules.

38. The method according to claim 1, wherein the target polynucleotide is derived from a human being, an animal, bacteria, virus, fungi, protozoa and/or plant.

39. The method according to claim 1, wherein the target polynucleotide is isolate from a sample from a human or animal body.

40. The method according to claim 1, wherein the target polynucleotide is isolate from humans, animals, birds, insects, plants, algae, fungi's, yeast, viruses, bacteria and phages, multi-cellular and mono-cellular organisms.

41. The method according to claim 1, wherein the target polynucleotide is isolate from faeces, blood, semen, cerebrospinal fluid, sputum, vaginal fluid, urine, saliva, hair, other bodily fluids, tissue samples, whole organs, sweat, tears, skin cells, hair, bone, teeth or appropriate fluid or tissue from personal items (e.g. toothbrush, razor, etc.) or from samples (e.g. sperm or biopsy tissue or liquid) or other sub-structures of humans or animals.

42. The method according to claim 1, wherein the total number of different target polynucleotide sequences captured are selected from the group consisting of 1, 2-5, 5-10, 10-15, 15-20, 20-25, 25-30, 30-35, 35-40, 40-45, 45-50, 50-55, 55-60, 60-65, 65-70, 70-75, 75-80, 80-85, 85-90, 95-100, 100-150, 150-200, 200-300, 300-500, 500-1000 and more than 1000 different, or any combination of these intervals.

43. The method according to claim 1 for diagnosing a genetic disease, wherein the genetic disease is associated with the target polynucleotide.

44. (canceled)

45. The method according to claim 43 wherein the disease to be diagnosed is selected from the group consisting of CADASIL syndrome; Carboxylase Deficiency, Multiple, Late-Onset; Cerebelloretinal Angiomatosis, familial; Crohn's disease, fibrostenosing; Deficiency disease, Phenylalanine Hydroxylase; Fabry disease; Hereditary coproporphyria; Incontinentia pigmenti; Microcephaly; Polycystic kidney disease; Siderius X-linked mental retardation syndrome caused by mutations in the PHF8 gene and achondroplasia.

46. The method according to claim 43, wherein the disease to be diagnosed is cancer.

47.-50. (canceled)

51. The method according to claim 1, wherein the sample is a feed, soil, food or drinking water.

52.-54. (canceled)

55. An oligonucleotide probe consisting of 15 to 35 nucleotides, wherein the oligonucleotide probe comprises at least 2 intercalator molecules which fit morphologically into abasic sites of a complementary polynucleotide target, and wherein the nucleotides adjacent to the intercalator molecules of the oligonucleotide probe are complementary to the polynucleotide target.

56.-79. (canceled)

80. A complex comprising an oligonucleotide probe and a complementary polynucleotide target comprising at least two abasic sites,
wherein the oligonucleotide probe has a length of 15 to 35 nucleotides and comprises at least two intercalator molecules inserted into the backbone-structure of said oligonucleotide probe, and wherein said at least two intercalator molecules fit morphologically into the abasic sites of the complementary polynucleotide target.

81.-104. (canceled)
